(19) United States
(12) Patent Application Publication
(10) Pub. No.: US 2015/0377879 A1
(43) Pub. Date: Dec. 31, 2015

(54) PEPTOIDS THAT BIND SPECIFIC ANTIGENS

(71) Applicant: THE SCRIPPS RESEARCH INSTITUTE, La Jolla, CA (US)

(72) Inventors: Thomas Kodadek, Jupiter, FL (US); Bindu Raveendra, Jupiter, FL (US); Hao Wu, Jupiter, FL (US); Roberto Baccala, San Diego, CA (US); Argyrios Theofilopoulos, San Diego, CA (US)

(73) Assignee: THE SCRIPPS RESEARCH INSTITUTE, La Jolla, CA (US)

(21) Appl. No.: 14/767,942

(22) PCT Filed: Feb. 13, 2014
(86) PCT No.: PCT/US14/16222

§ 371 Date: Aug. 14, 2015

Related U.S. Application Data
(60) Provisional application No. 61/765,202, filed on Feb. 15, 2013.

(51) Int. Cl. G01N 33/564; C07K 7/06

(52) U.S. Cl. CPC G01N 33/564; C07K 7/06; G01N 2800/285; G01N 2650/00

(57) ABSTRACT

Combinatorial libraries were generated providing a vast number of diverse peptoid ligands. From these libraries, ligands were identified which specifically bind molecules associated with autoimmune diseases, such as antibodies specific to aquaporin-4 (AQP4), binding of which to AQP4 causes the autoimmune disease, Neuoromyelitis Optica. Methods of generating peptoid libraries and for diagnosing Neuromyelitis Optica are also provided.

### CROSS-REFERENCE TO RELATED APPLICATIONS

The present application claims priority to U.S. provisional application Ser. No. 61/765,202 filed on Feb. 15, 2013, which is incorporated herein by reference in its entirety.

### FIELD OF THE INVENTION

Embodiments of the invention are directed to compositions for identification of autoantigens, or other disease related antigens, such as, biomarkers. Methods of producing combinatorial libraries of ligands that specifically bind molecules associated with disease are provided.

### BACKGROUND

Neuromyelitis optica (NMO) is a rare, autoimmune demyelinating disease that can result in blindness and paralysis (Wingerchuk et al., 2006, Neurology 78:665-671). A major breakthrough in the understanding of NMO was the discovery that most NMO patients have high levels of circulating IgG autoantibodies against a water channel protein aquaporin 4 (AQP4)(Lennon et al., 2004, Lancet 364, 2106-2112) expressed on the surface of astrocytes in the central nervous system (CNS). There is evidence that these autoantibodies fix complement on the surface of certain AQP4-expressing cells (Crane et al., 2011, The Journal of Biological Chemistry 286:16516-16524), resulting in tissue injury (Papadopoulos and Verkman, 2012, Lancet Neurol 11, 535-544). Currently, anti-AQP4 autoantibodies may be detected by a variety of methods: ELISA against recombinant AQP4 protein, tissue-based immunofluorescence, AQP4-transfected cell-based assays, fluorescence immunoprecipitation assays, and flow cytometric assays. The target epitopes recognized by AQP4 autoantibodies in these assays include determinants on the three extracellular loops (Pisani et al., 2011, J. Biol. Chem. 286:9216-9224); however, the sequence and conformational determinants remain unresolved due to the use of polyclonal patient serum and the limited characterization of the AQP4 protein target.

Despite the high diagnostic specificity of these multiple assays, approximately 25% (Waters et al., 2012, Neurol. 78:665-671) of patients with clinical NMO lack readily detectable anti-AQP4 antibodies. These patients may have low-titer, low affinity anti-AQP4 antibodies, or may produce autoantibodies against alternative CNS targets. Misdiagnosis of these patients may lead to unnecessary diagnostic studies and inappropriate therapy and highlights the need for further work on the discovery of biomarkers for the disease.

### SUMMARY

Embodiments are directed to ligands that specifically bind molecules associated with diseases or disorders comprising: autoimmune diseases, cancer, cardiovascular diseases, inflammation, inflammatory diseases and the like. Combinatorial libraries are generated which provide a vast number of diverse ligands. Methods of generating the libraries are provided.

In some embodiments, the peptoids comprise at least five variable positions which can give rise to a diverse library of at least 10⁵. In embodiments the peptoids comprise a general structural formula (I):

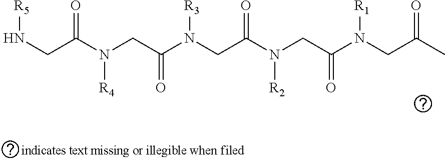

Wherein

R₁, R₂, R₃, R₄, R₅ independently comprise one or more groups derived from amines comprising:

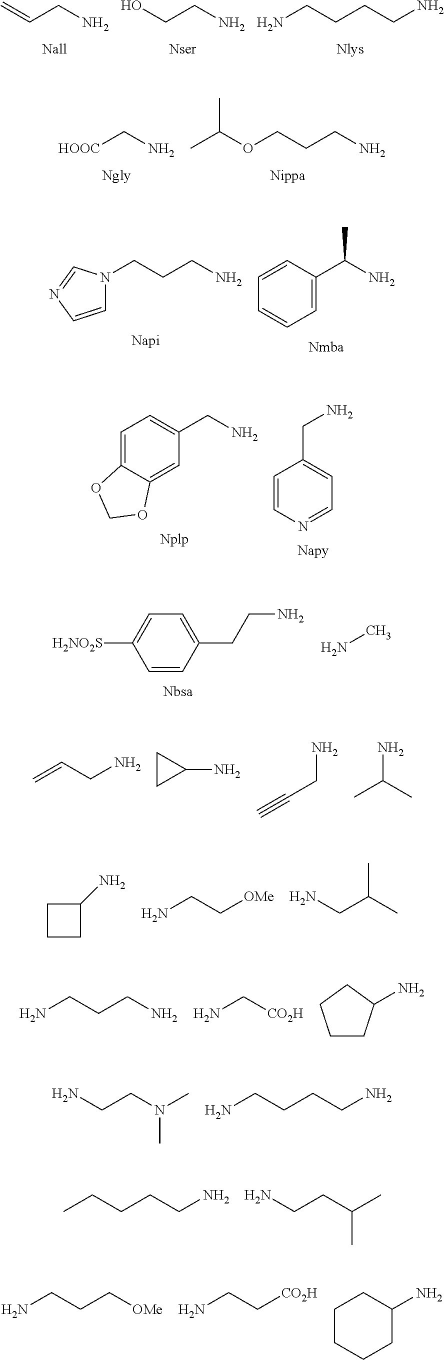

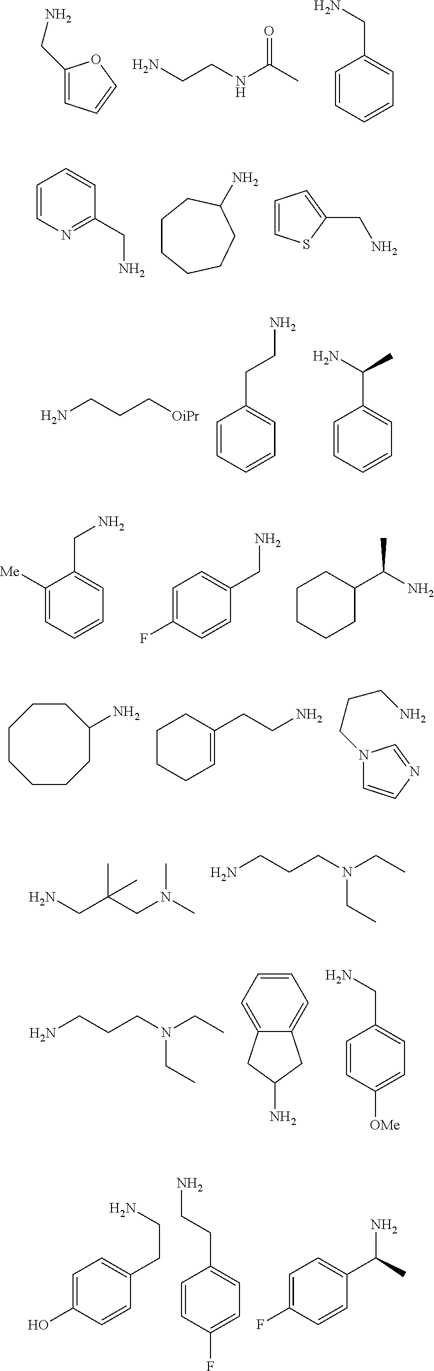

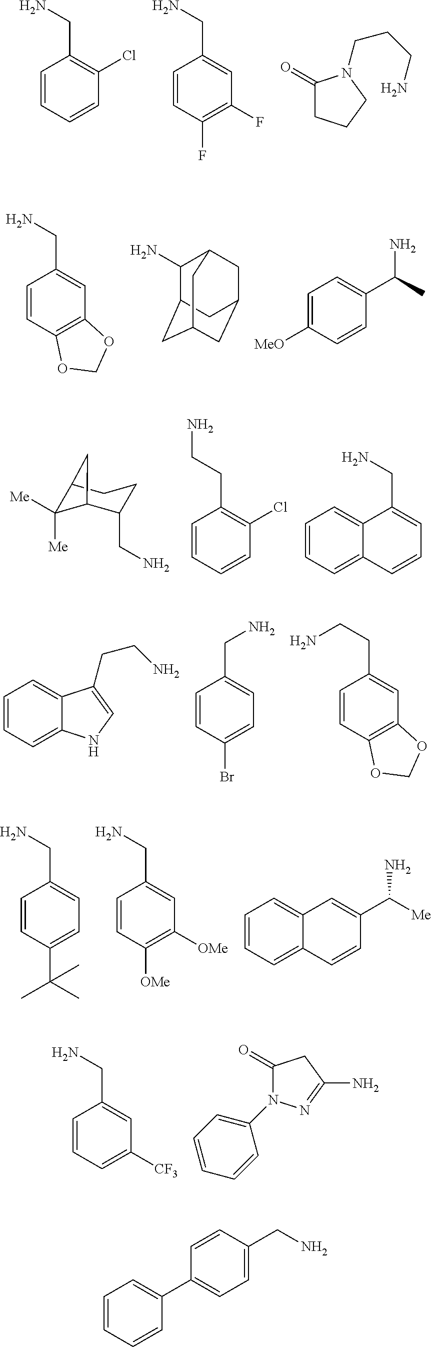

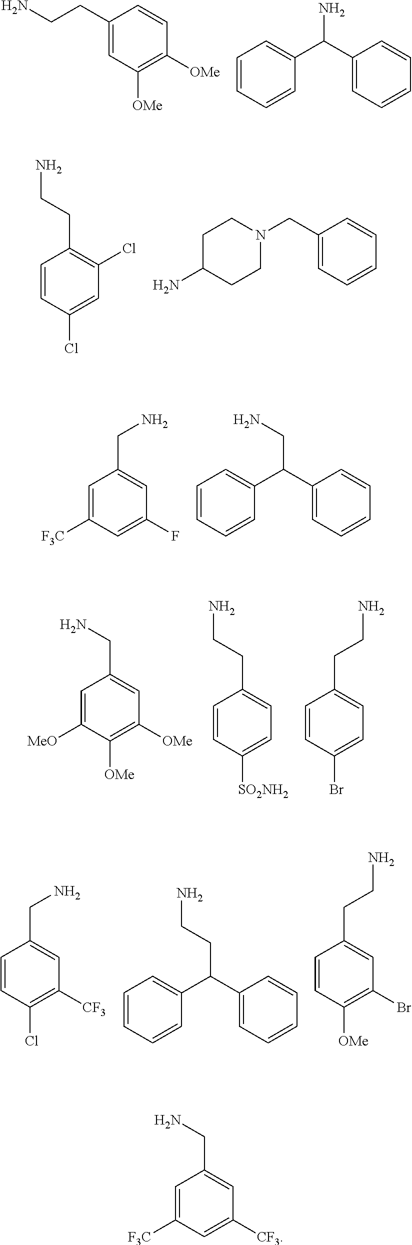

Other aspects of the invention are described infra.

### BRIEF DESCRIPTION OF THE DRAWINGS

FIGS. 1A-1D show the screening a combinatorial peptoid library for ligands to NMO-specific antibodies. FIG. 1A: Schematic depiction of the screening strategy. An OBOC library was pre-screened with control serum to eliminate the beads that can bind to the antibodies present at high levels in control (non-NMO) sera. The denuded library was then screened against a pool of NMO serum high in anti-AQP4 antibodies. In both the pre-screen and the NMO screen, antibody-binding beads were visualized using a red quantum dot-conjugated secondary antibody. The hits were identified by tandem mass spectrometry. FIG. 1B: General structure of the 8-mer peptoid in the library used for the screening of NMO sera. The invariant linker is shown in black and the variable region in blue with side chains substituents (from the amines) in red. FIG. 1C: List of amines used in the solid-phase synthesis of the library. FIG. 1D: A representative photomicrograph of the library under the fluorescent microscope after incubation with the serum followed by hybridization of antihuman secondary antibody conjugated with Qdot 655. The beads were irradiated through a DAPI filter. The beads with the red halo are the hits.

FIGS. 2A-2C show the validation of bead screening hits by microarray analysis. Results of hybridizing serum samples or purified antibodies to the array, followed by washing and subsequent addition of a red-labeled secondary antibody (see Methods). The screen shots from the microarray scanner are shown. FIG. 2A: Analysis of serum samples from a control individual who did not have NMO (Normal) and NMO patients that tested either positive (Anti-AQP4⁺) or negative (Anti-AQP4−) for complement-mediated cell killing of cells expressing AQP4. FIG. 2B: Analysis of purified monoclonal anti-AQP4 antibody and control antibody. NMOP8 binds the secondary antibody directly. DNP=dinitrophenol. FIG. 2C: Chemical structures of the peptoids that showed significant affinity for NMO-specific antibodies in one of the samples. The structure of the linker is shown in FIGS. 1A-1D.

FIG. 3 shows that peptoid NMOP6 is an AQP4 antigen surrogate. The graph depicts the level of signal on an array for the peptoid and serum samples indicated. MS=multiple sclerosis. The serum samples indicated were either applied to the array directly or after being incubated with immobilized AQP4 or, as a control, BSA. NMOP6S is a scrambled version of NMOP6. Note that the terms “anti-AQP4 AB⁺” and “anti-AQP4 Ab⁻” connote serum samples that tested positive and negative, respectively, in the assay for complement-mediated killing of AQP4-expressing cells.

FIG. 4 shows the results from a blinded analysis of 15 serum samples. 15 serum samples were analyzed in a blinded fashion on arrays of the type shown in FIGS. 2A-2C. A heat map representing the intensities observed on the arrays is shown. The samples were called NMO if any of the peptoids (other than NMOP8, which binds the secondary antibody directly) displayed a clear signal above background. The calls, made prior to unblinding, are shown in red at the bottom of the heat map and the identities of the samples are shown in black below the calls. C=control, N+=NMO serum that tested positive in the complement-mediated cell-killing assay, N−=NMO serum that tested negative in the cell-killing assay.

FIGS. 5A-5D show the Tandem Mass spectrometric identification of Peptoid Hits. FIG. 5A shows the mass spectrum of one of the hits isolated from NMO serum screening using OBOC peptoid library. FIG. 5B: MS-MS spectrum of the hit to determine the sequence of the compound. 495 is the linker mass. FIG. 5C: Mass spectrum of one of the hits resynthesized with a Cysteine residue at the C-terminus in the soluble form to immobilize on microarray slide and FIG. 5D: MS-MS of the compound. 514 is the linker mass.

FIGS. 6A, 6B show the validation of NMO screening hits on Microarray. FIG. 6A: The anti-AQP4 Ab⁺ NMO sera hits were validated on the microarray using individual anti-AQP4 Ab+ NMO serum samples (#30, #35, #68, #84). All the anti-AQP4 Ab+ NMO sera tested here showed consistent binding signal with NMOP6 and three out of four showed weak binding to NMOP2. FIG. 6B: Binding pattern of anti-AQP4 Ab− NMO Sera (#15, #31, #74 and #81) on the peptoid array. NMOP6, NMOP2, NMOP4, NMOP5, NMOP7 and NMOP9 lighted up in some of the samples, but the intensity of binding varied from serum to serum. Note that the terms “anti-AQP4 Ab⁺” and “anti-AQP4 Ab⁻” are meant to connote serum samples that tested positive and negative, respectively, in the assay for complement-mediated killing of AQP4-expressing cells.

FIG. 7 shows an anti-AQP4 antibody (Ab) depletion experiment. The array shows the binding of the pure antibody/serum before and after depletion of the anti-AQP4 antibody. The antibody was depleted by passing through an AQP4 protein-immobilized column, and BSA-immobilized column was used as a control. After passing the serum through the AQP4-column, the binding of NMOP6 completely disappeared in all serum samples, and the same was observed with the monoclonal anti-AQP4 rAb. In contrast, the control BSA-column had no effect on peptoid binding. The peptoid hits NMOP5, NMOP7 and NMOP9 also showed decreased binding intensity after passing through the AQP4-column, but the binding signal was not completely eliminated. The BSA-column showed similar results, indicating that there is a non-specific interaction between the serum and the column resulting in the removal of certain types of sticky antibodies from the serum.

FIGS. 8A, 8B show the analysis of serum samples from patients with other diseases and additional healthy controls. Shown are the microarray images resulting from hybridization of serum samples collected from patients with: FIG. 8A-Narcolepsy (either with psychotic behavior (NP) or non-psychotic behavior (NS)), normal healthy controls (NC). FIG. 8B: lupus (SLE) and Alzheimer's disease.

FIG. 9 is a graph showing the results from a competition ELISA using NMO serum. NMOP6-dextran competes to the signal of case sera but not of normal at 1 μM. ADP3-dextran does not compete except to one case sera (AC3252). NMOP6 and ADP3 monomers do not compete at 100 μM.

FIG. 10 is a graph showing results from a microarray-based analysis of 15 blinded samples using NMOP6 immobilized on a microarray.

FIG. 11 is a selected microarray image of results obtained from a NMOP6 competition assay.

FIG. 12 is a graph showing results from an ELISA using immobilized NMOP6 and a soluble competitor.

FIG. 13 is a schematic representation showing the different modes of immunoglobulin binding.

### DETAILED DESCRIPTION

In general embodiments, library screening of diverse ligands identifies markers associated with disease. In particular, generation of diverse libraries screened against patient serum samples detected molecules associated with autoimmune-mediated disease.

As an illustrative example of the utility of the ligands generated and the screening methods utilized in identifying biomarkers of diseases, sera from patients with Neuromyelitis optica (NMO) was used. It is to be understood that this is merely for illustrative purposes and is not meant to be limiting or construed as limiting. From the perspective of validation of this novel method for biomarker discovery in a human disease, NMO is an attractive system as isolation of peptoids that bind to anti-AQP4 autoantibodies, provides a clear validation of the approach. Screening of 100,000 peptoids using a second-generation bead-based screening approach indeed yielded several peptoid ligands for the antigen-binding site of anti-AQP4 antibodies. These peptoids distinguish between NMO patient serum and serum from healthy controls or patients with MS, Alzheimer's Disease, narcolepsy and lupus with high accuracy.

### DEFINITIONS

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The terms, “ligand” and “ligands” as used herein refers to a peptoid encompassed by the generic formulae disclosed herein, any subgenus of those generic formulae, and any forms of the ligands within the generic and subgeneric formulae. Unless specified otherwise, the term further includes the racemates and stereoisomers, of the ligand or ligands.

The terms “solvate” or “solvates” of a compound refer to those compounds, where compounds is as defined above, that are bound to a stoichiometric or non-stoichiometric amount of a solvent. Solvates of a compound includes solvates of all forms of the compound. Preferred solvents are volatile, non-toxic, and/or acceptable for administration to humans in trace amounts. Suitable solvates include distilled and pyrogen-free water.

The term “substituted” as used herein refers to substitution with the named substituent or substituents, multiple degrees of substitution being allowed unless otherwise stated.

“Autoimmune disease” as used herein refers to any group of disorders in which tissue injury is associated with humoral or cell-mediated responses to the body's own constituents.

The term “specifically binds” to a target molecule, such as for example, an antibody or a polypeptide is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. For example, an antibody “specifically binds” or “preferentially binds” to a target if it binds with greater affinity, avidity, more readily, and/or with greater duration than it binds to other substances. It is also understood by reading this definition that; for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

“Diagnostic” or “diagnosed” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

“Treatment” is an intervention performed with the intention of preventing the development or altering the pathology or symptoms of a disorder. Accordingly, “treatment” refers to both therapeutic treatment and prophylactic or preventative measures. “Treatment” may also be specified as palliative care. Those in need of treatment include those already with the disorder as well as those in which the disorder is to be prevented. Accordingly, “treating” or “treatment” of a state, disorder or condition includes: (1) preventing or delaying the appearance of clinical symptoms of the state, disorder or condition developing in a human or other mammal that may be afflicted with or predisposed to the state, disorder or condition but does not yet experience or display clinical or subclinical symptoms of the state, disorder or condition; (2) inhibiting the state, disorder or condition, i.e., arresting, reducing or delaying the development of the disease or a relapse thereof (in case of maintenance treatment) or at least one clinical or subclinical symptom thereof; or (3) relieving the disease, i.e., causing regression of the state, disorder or condition or at least one of its clinical or subclinical symptoms. The benefit to an individual to be treated is either statistically significant or at least perceptible to the patient or to the physician.

The terms “patient” or “individual” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters; and primates.

As used herein, “biological samples” or “samples” include solid and body fluid samples. The biological samples used in the present invention can include cells, protein or membrane extracts of cells, blood or biological fluids such as ascites fluid or brain fluid (e.g., cerebrospinal fluid). Examples of solid biological samples include, but are not limited to, samples taken from tissues of the central nervous system, bone, breast, kidney, cervix, endometrium, head/neck, gallbladder, parotid gland, prostate, pituitary gland, muscle, esophagus, stomach, small intestine, colon, liver, spleen, pancreas, thyroid, heart, lung, bladder, adipose, lymph node, uterus, ovary, adrenal gland, testes, tonsils, thymus and skin, or samples taken from tumors. Examples of “body fluid samples” include, but are not limited to blood, serum, semen, prostate fluid, seminal fluid, urine, feces, saliva, sputum, mucus, bone marrow, lymph, and tears.

The term “high-throughput screening” or “HTS” refers to a method drawing on different technologies and disciplines, for example, optics, chemistry, biology or image analysis to permit rapid, highly parallel biological research and drug discovery. HTS methods are known in the art and they are generally performed in multiwell plates with automated liquid handling and detection equipment; however it is also envisioned that the methods of the invention may be practiced on a microarray or in a microfluidic system.

A “dextran” is a polymer comprising glucose units, also referred to as a polyglucose, and contains at least 50% of continuous alpha 1,6 glucosidic bonds. Dextrans of a wide variety of structures and molecular weights have been known for many years. Dextrans are produced by lactic acid bacteria growing on a sucrose substrate; for example Leuconostoc, Lactococcus, Streptococcus, Weisella, and Lactobacillus. The enzymes involved in their synthesis are glucansucrases which produce glucans and release fructose from sucrose substrates. The terms dextran, native dextran and high molecular weight dextran as used herein are synonyms. Dextran often has an average molecular weight above 1000 kDa. The dextran molecules can also be modified dextran molecules, for example, may have a detectable label, biotin, a synthetic molecule, an organic or inorganic molecule, nucleic acid molecule, amine or amine derived group, any chemical group, and the like.

“Solid support” or “support” surface refers to any substrate having a surface to which molecules may be attached, directly or indirectly, through either covalent or non-covalent bonds. The solid support may include any substrate material that is capable of providing physical support for the probes that are attached to the surface. The material is generally capable of enduring conditions related to the attachment of the probes to the surface and any subsequent treatment, handling, or processing encountered during the performance of an assay. The materials may be naturally occurring, synthetic, or a modification of a naturally occurring material. Suitable solid support materials may include silicon, graphite, mirrored surfaces, laminates, ceramics, plastics (including polymers such as, e.g., poly(vinyl chloride), cyclo-olefin copolymers, polyacrylamide, polyacrylate, polyethylene, polypropylene, poly(4-methylbutene), polystyrene, polymethacrylate, poly(ethylene terephthalate), polytetrafluoroethylene (P or TEFLON™), nylon, poly(vinyl butyrate)), germanium, gallium arsenide, gold, silver, etc., either used by themselves or in conjunction with other materials. Additional rigid materials may be considered, such as glass, which includes silica and further includes, for example, glass that is available as Bioglass. Other materials that may be employed include porous materials, such as, for example, controlled pore glass beads. Any other materials known in the art that are capable of having one or more functional groups, such as any of an amino, carboxyl, thiol, or hydroxyl functional group, for example, incorporated on its surface, are also contemplated.

The material used for a solid support may take any of a variety of configurations ranging from simple to complex. The solid support can have any one of a number of shapes, including a strip, plate, disk, rod, particle, including bead, tube, well, and the like. Usually, the material is relatively planar such as, for example, a slide, though it can be spherical, such as, for example, a bead, or cylindrical (e.g., a column). In many embodiments, the material is shaped generally as a rectangular solid. Multiple predetermined arrangements such as, e.g., arrays of probes, may be synthesized on a sheet, which is then diced, i.e., cut by breaking along score lines, into single array substrates. Exemplary solid supports that may be used include microtiter wells, microscope slides, membranes, paramagnetic beads, charged paper, Langmuir-Blodgett films, silicon wafer chips, flow through chips, and microbeads.

The surface of the solid support is usually the outer portion of the substrate material that forms the solid support. The surface of the solid support onto which the probes are bound may be smooth or substantially planar, or have irregularities, such as depressions, grooves, elevations, or other textures. The surface may be modified with one or more different layers of compounds that serve to modify the properties of the surface in a desirable manner. In various embodiments, such surface modification layers, when present, can generally range in thickness from a monomolecular thickness to about 1 mm, or from a monomolecular thickness to about 0.1 mm, or from a monomolecular thickness to about 0.001 mm.

Surface modification layers of interest include inorganic and organic layers, such as metals, metal oxides, polymers, small organic molecules, and the like. Polymeric layers of interest include methacrylate copolymers, polyacrylamides, polysaccharides, phospholipids, polyurethanes, polyesters, polycarbonates, polyureas, polyamides, polyethylene amines, polyarylene sulfides, polysiloxanes, polyimides, polyacetates, and the like, where the polymers may be hetero- or homo-polymeric, and may or may not have separate functional moieties attached thereto (for example, conjugated moieties). Other surface modifications of interest include three-dimensional networks, such as hydrogels, for example. Any suitable hydrogel known in the art may be used.

Peptoid Ligands

The discovery of diagnostically useful serum biomarkers is of great interest in translational science. In many diseases, it is likely that an adaptive immune response produces disease-specific antibodies that would be excellent candidates for such biomarkers. However, a major impediment to mining the immune system for useful biomarkers is the assumption that only the native antigen will bind to disease-specific antibodies with sufficient affinity and selectivity to retain them from the serum and allow their levels to be measured. Prior to this invention, the discovery of such antigens has proven difficult in most diseases.

Embodiments of the invention are directed to the identification of biomarkers of disease. In one embodiment, the disease is an autoimmune disease. The biomarkers may be any molecule associated with an autoimmune disease, such as, for example: antibodies directed to autoantigens, T cell receptors directed to autoantigens and the like.

In embodiments, a method of identifying ligands which specifically bind to biomarkers associated with autoimmune diseases, the method comprising: providing a compound library of candidate ligands wherein each ligand is coupled to a support; contacting the library with a control sample to identify ligands associated with non-specific binding; removing the ligands associated with non-specific binding; and contacting the remaining ligands with a test sample and a labeled secondary antibody. The support to which the ligands are coupled can vary, for example: a bead, a chip, a filter, a dipstick, a membrane, a polymer matrix or a well. Examples of autoimmune diseases comprise, without limitation: acute disseminated encephalomyelitis (ADEM), acute necrotizing hemorrhagic leukoencephalitis, Addison's disease, agammaglobulinemia, allergic asthma, allergic rhinitis, alopecia greata, amyloidosis, ankylosing spondylitis, anti-GBM/anti-′I′BM-nephritis, antiphospholipid syndrome (APS), autoimmune aplastic anemia, autoimmune dysautonomia, autoimmune hepatitis, autoimmune hyperlipidemia, autoimmune immunodeficiency, autoimmune inner ear disease (AIED), autoimmune myocarditis, autoimmune pancreatitis, autoimmune retinopathy, autoimmune thrombocytopenic purpura (ATP), autoimmune thyroid disease, axonal & neuronal neuropathies, Balo disease, Behcet's disease, bullous pemphigoid, cardiomyopathy, Castlemen disease, celiac sprue (non-tropical), Chagas disease, chronic fatigue syndrome, chronic inflammatory demyelinating polyneuropathy (CIDP), chronic recurrent multifocal ostomyelitis (CRMO), Churg-Strauss syndrome, cicatricial pemphigoid/benign mucosal pemphigoid, Crohn's disease, Cogan's syndrome, cold agglutinin disease, congenital heart block, coxsackie myocarditis, CREST disease, essential mixed cryoglobulinemia, demyelinating neuropathies, dermatomyositis, Devic's disease (neuromyelitis optica), discoid lupus, Dressler's syndrome, endometriosis, eosinophillic fasciitis, erythema nodosum, experimental allergic encephalomyelitis, Evan's syndrome, fibromyalgia, fibrosing alveolitis, giant cell arteritis (temporal arteritis), glomerulonephritis, Goodpasture's syndrome, Grave's disease, Guillain-Barre syndrome, Hashimoto's encephalitis, Hashimoto's thyroiditis, hemolytic anemia, Henock-Schoniein purpura, herpes gestationis, hypogammaglobulinemia, idiopathic thrombocytopenic purpura (ITP), IgA nephropathy, immunoregulatory lipoproteins, inclusion body myositis, insulin-dependent diabetes (type 1), interstitial cystitis, juvenile arthritis, juvenile diabetes, Kawasaki syndrome, Lambert-Eaton syndrome, leukocytoclastic vasculitis, lichen planus, lichen sclerosus, ligneous conjunctivitis, linear IgA disease (LAD), Lupus (SLE), Lyme disease, Meniere's disease, microscopic polyangitis, mixed connective tissue disease (MCTD), Mooren's ulcer, Mucha-Habermann disease, multiple sclerosis, myasthenia gravis, myositis, narcolepsy, neuromyelitis optica (Devic's), neutropenia, ocular cicatricial pemphigoid, optic neuritis, palindromic rheumatism, PANDAS (Pediatric Autoimmune Neuropsychiatric Disorders Associated with Streptococcus), paraneoplastic cerebellar degeneration, paroxysmal nocturnal hemoglobinuria (PNH), Parry Romberg syndrome, Parsonnage-Turner syndrome, pars plantis (peripheral uveitis), pemphigus, peripheral neuropathy, perivenous encephalomyelitis, pernicious anemia, POEMS syndrome, polyarteritis nodosa, type I, II & III autoimmune polyglandular syndromes, polymyalgia rheumatic, polymyositis, postmyocardial infarction syndrome, postpericardiotomy syndrome, progesterone dermatitis, primary biliary cirrhosis, primary sclerosing cholangitis, psoriasis, psoriatic arthritis, idiopathic pulmonary fibrosis, pyoderma gangrenosum, pure red cell aplasis, Raynaud's phenomena, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Slogren's syndrome, sperm and testicular autoimmunity, stiff person syndrome, subacute bacterial endocarditis (SBE), sympathetic ophthalmia, Takayasu's arteritis, temporal arteritis/giant cell arteries, thrombocytopenic purpura (TPP), Tolosa-Hunt syndrome, transverse myelitis, ulcerative colitis, undifferentiated connective tissue disease (UCTD), uveitis, vasculitis, vesiculobullous dermatosis, vitiligo or Wegener's granulomatosis or, chronic active hepatitis, primary biliary cirrhosis, cadilated cardiomyopathy, myocarditis, autoimmune polyendocrine syndrome type I (APS-I), cystic fibrosis vasculitides, acquired hypoparathyroidism, coronary artery disease, pemphigus foliaceus, pemphigus vulgaris, Rasmussen encephalitis, autoimmune gastritis, insulin hypoglycemic syndrome (Hirata disease), Type B insulin resistance, acanthosis, systemic lupus erythematosus (SLE), pernicious anemia, treatment-resistant Lyme arthritis, polyneuropathy, demyelinating diseases, atopic dermatitis, autoimmune hypothyroidism, vitiligo, thyroid associated opthalmopathy, autoimmune coeliac disease, ACTH deficiency, dermatomyositis, Sjogren syndrome, systemic sclerosis, progressive systemic sclerosis, morphea, primary antiphospholipid syndrome, chronic idiopathic urticaria, connective tissue syndromes, necrotizing and crescentic glomerulonephritis (NCGN), systemic vasculitis, Raynaud syndrome, chronic liver disease, visceral leishmaniasis, autoimmune Cl deficiency, membrane proliferative glomerulonephritis (MPGN), prolonged coagulation time, immunodeficiency, atherosclerosis, neuronopathy, paraneoplastic pemphigus, paraneoplastic stiff man syndrome, paraneoplastic encephalomyelitis, subacute autonomic neuropathy, cancer-associated retinopathy, paraneoplastic opsoclonus myoclonus ataxia, lower motor neuron syndrome and Lambert-Eaton myasthenic syndrome.

In other embodiments, a disease to be detected and/or diagnosed comprises: cancer, inflammatory diseases or disorders, infectious diseases or disorders, cardiovascular diseases or disorders and the like. The type of molecules that are specifically bound by the peptoids vary and can be antibodies, peptides, biomarkers, viral antigens, bacterial and the like.

In one embodiment, a method of identifying ligands which specifically bind to biomarkers associated with autoimmune diseases, the method comprising: obtaining a one bead one compound library (OBOC) wherein each bead comprises a peptoid; contacting the OBOC library with a control sample to identify peptoids associated with non-specific binding; removing the beads having peptoids associated with non-specific binding; contacting the remaining OBOC with a test sample and a labeled secondary antibody. In some embodiments, the ligands bind to antibodies associated with autoimmune diseases.

In some embodiments, the peptoids are at least a 3-mer and comprise at least one variable position:

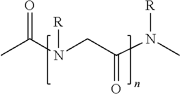

Wherein n=1-20, preferably, n=1-10, and R is an amine or a group derived from an amine comprising:

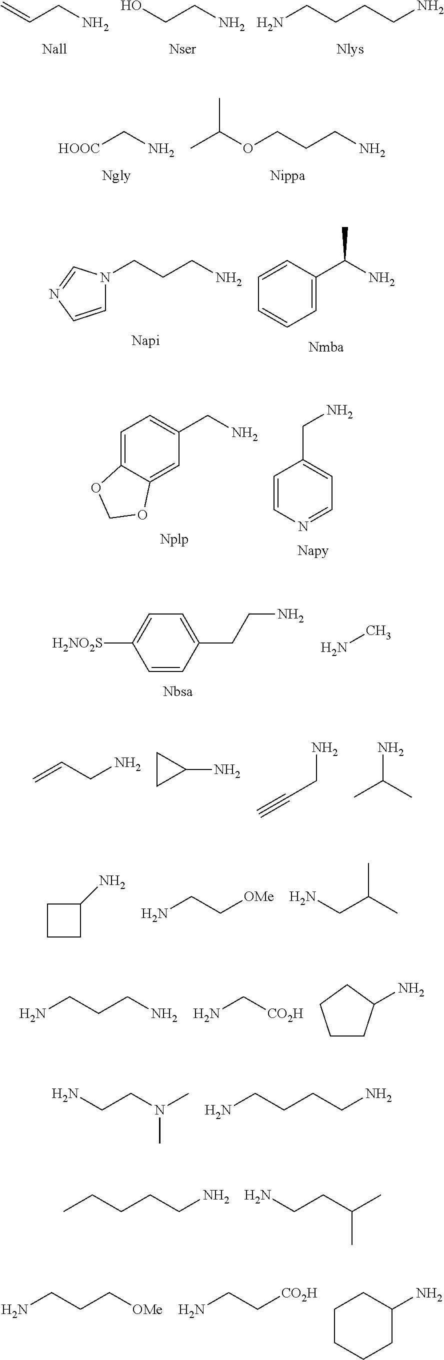

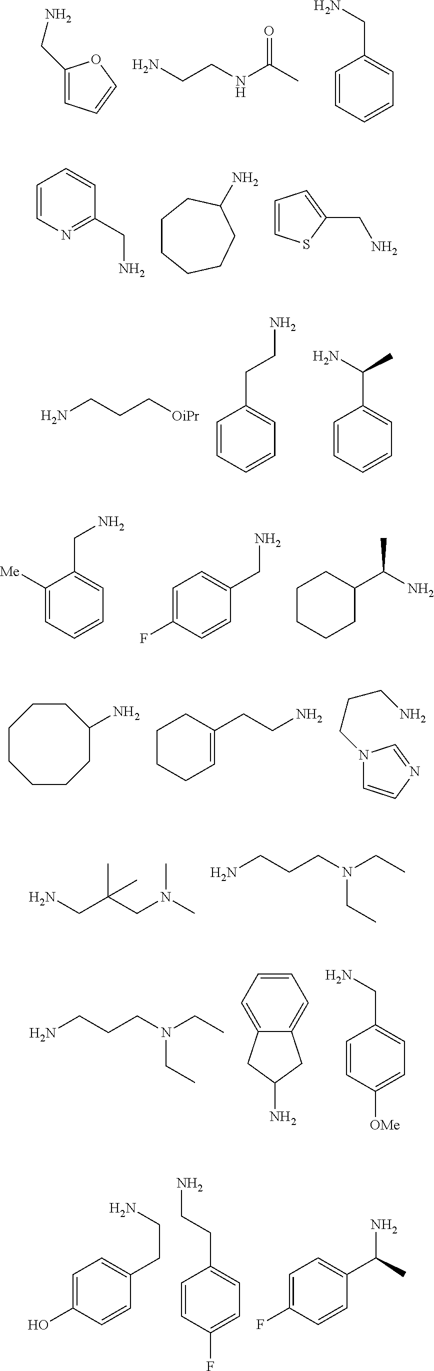

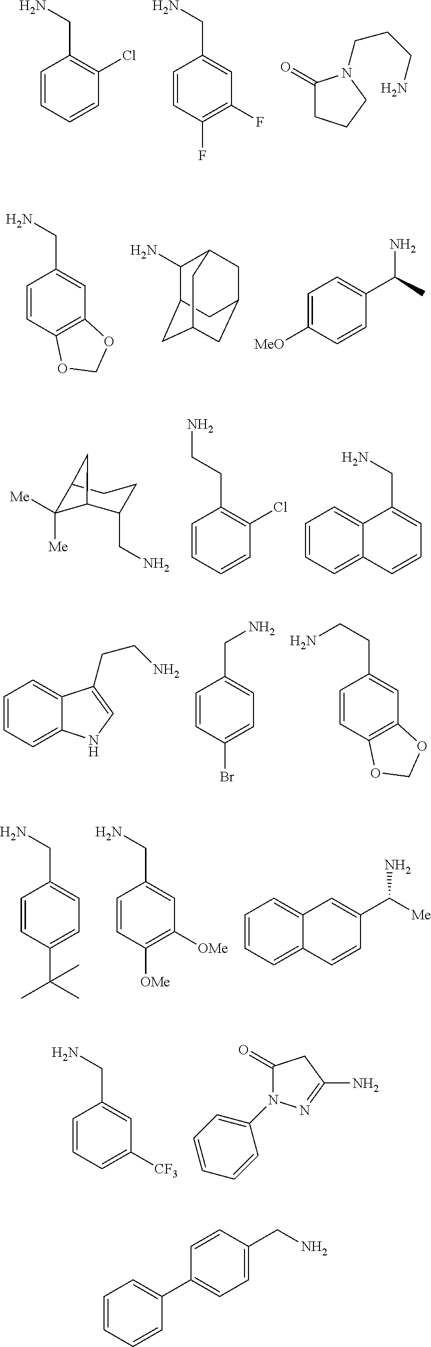

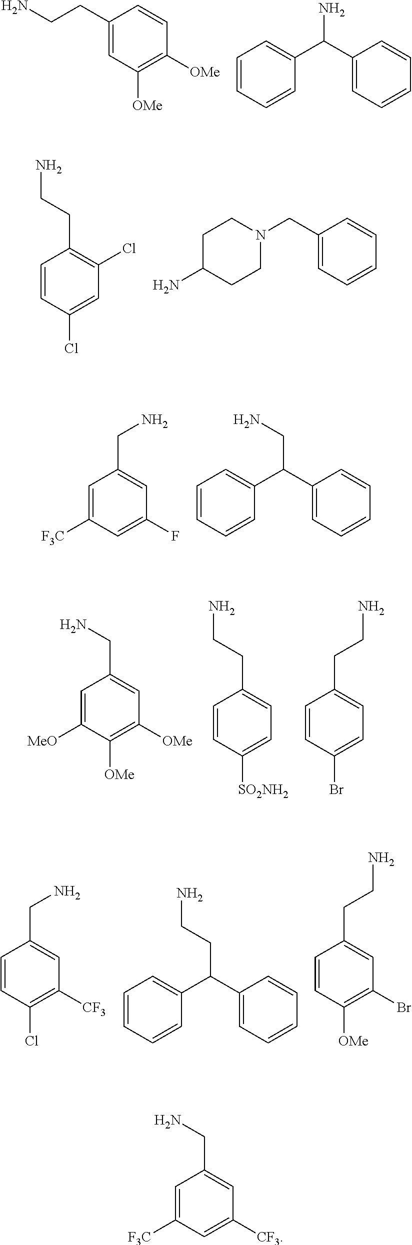

In embodiments, the peptoids comprise at least five variable positions which can give rise to a diverse library of at least 10⁵. In embodiments the peptoids comprise a general structural formula (I):

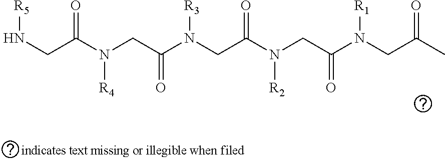

Wherein

R₁, R₂, R₃, R₄, R₅ independently comprise one or more groups derived from amines comprising:

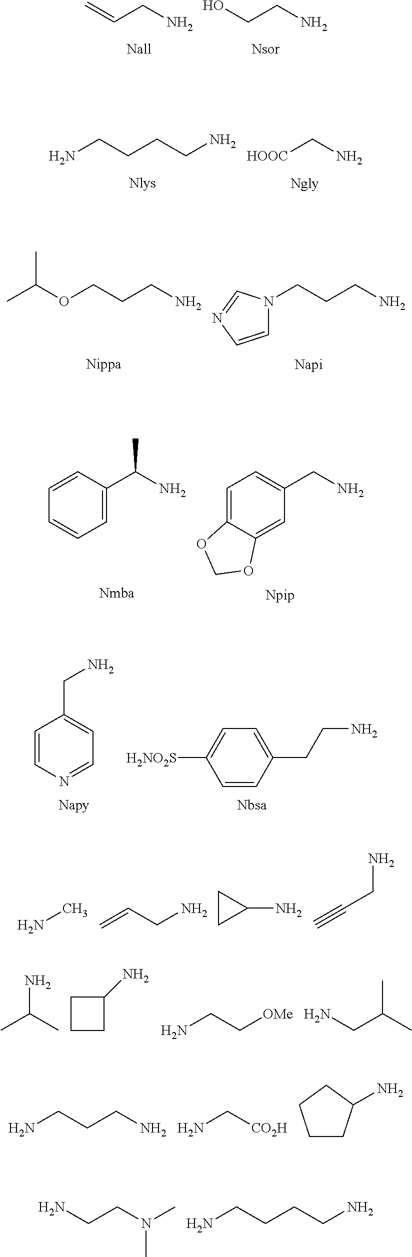

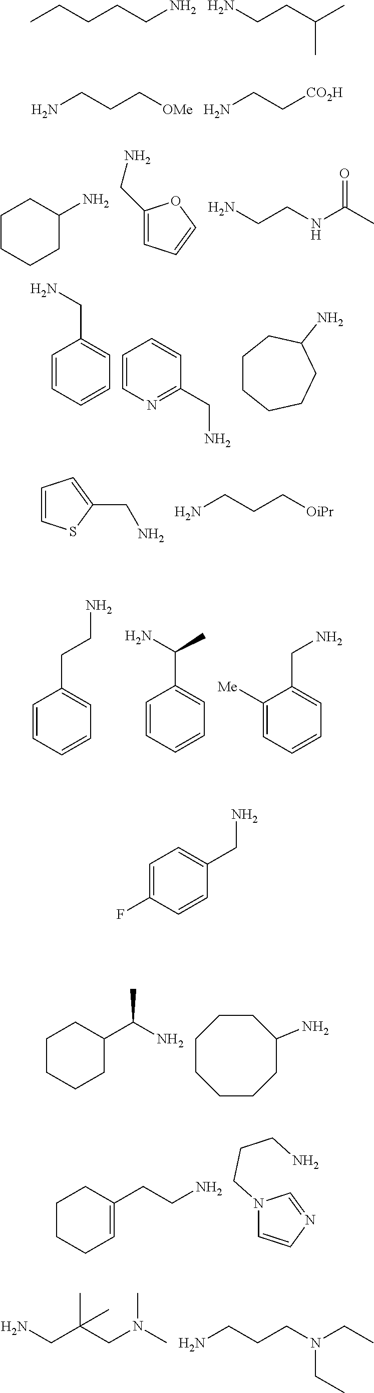

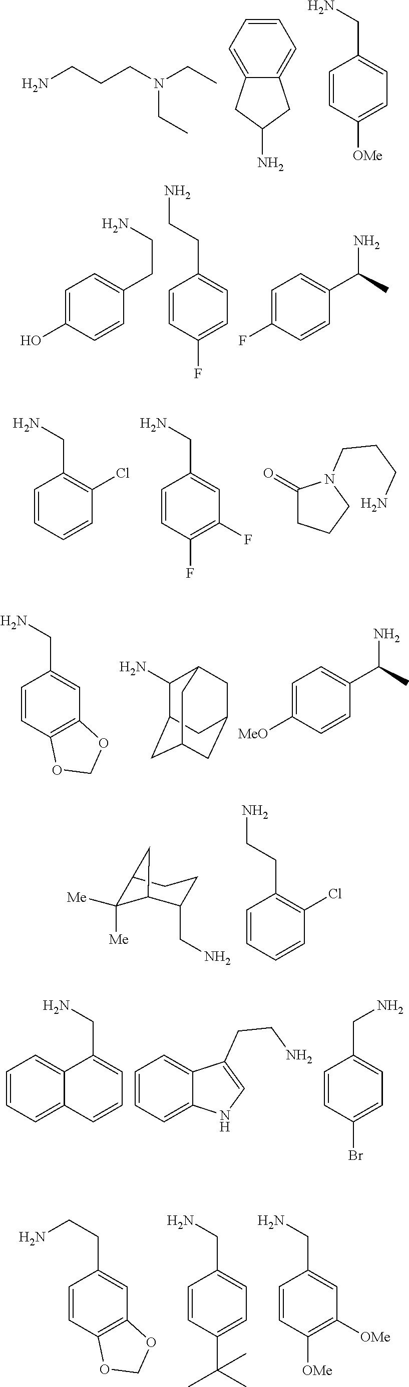

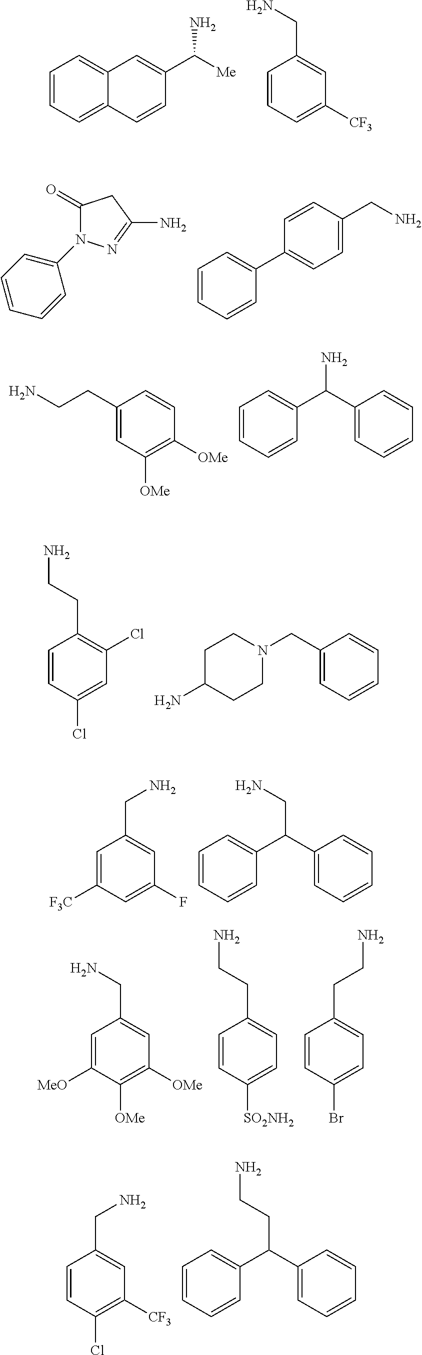

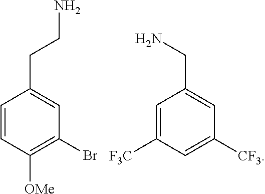

It is to be understood that the diversity of the combinatorial library can be increased or decreased based on the number of variable positions of the ligands (e.g. 3-mer, 4-mer, 5-mer, 6-mer, 7-mer, 8-mer etc.) and the types of substitutions.

In another embodiment, a peptoid comprises:

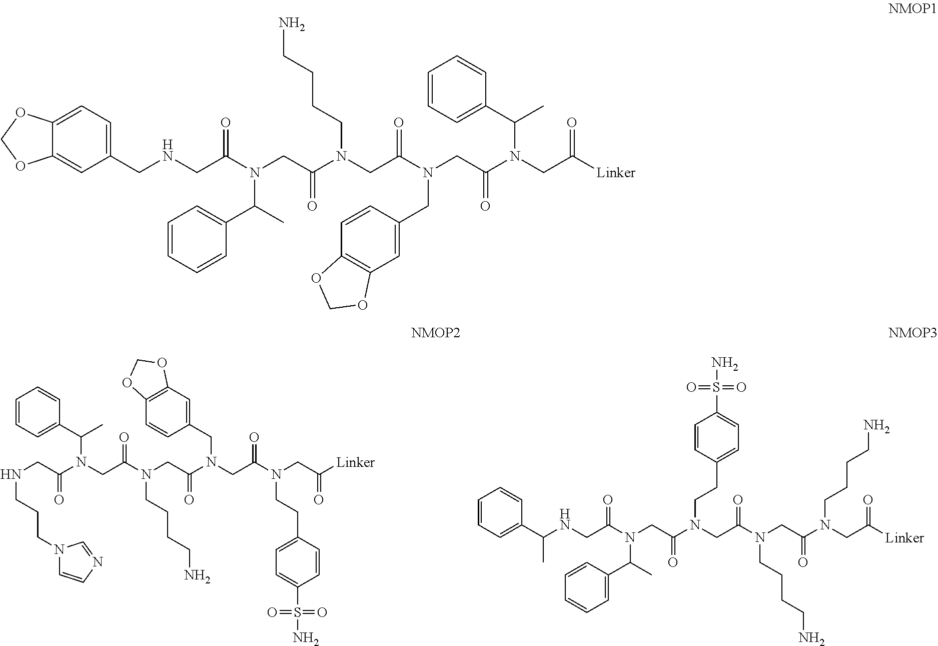

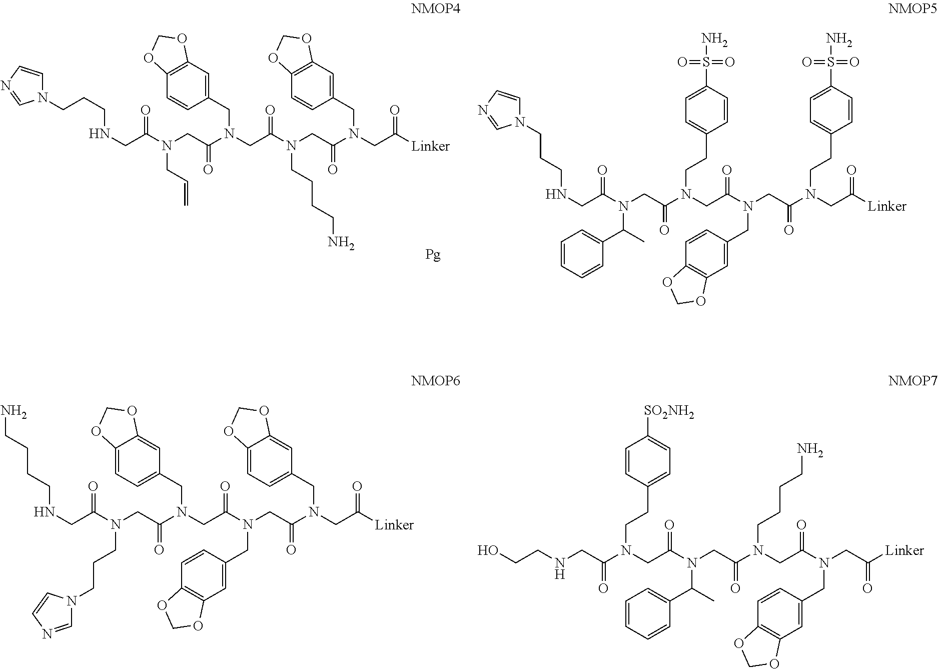

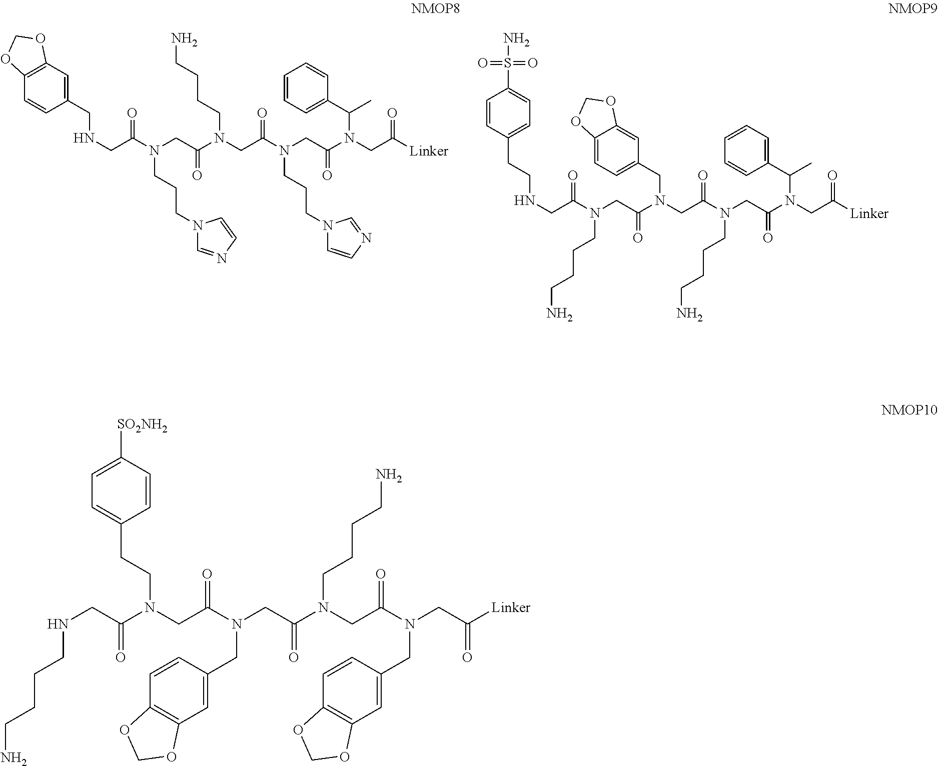

In another embodiment, the peptoids of formula (I) are attached to a bead via a linker molecule to give a compound of formula (II):

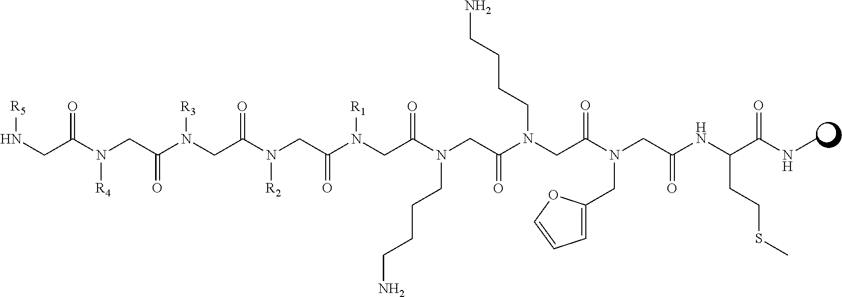

Wherein R₁-R₅ are identified as above. It should be understood that the invention is not restricted to the use of beads for coupling of the peptoids, but can include any acceptable substrate. Examples include, without limitation: bead, a chip, a filter, a dipstick, a membrane, a polymer matrix or a well

Similarly, the size and shape of the beads are not limited. However, in some embodiments, the average particle diameter of the bead is generally in the range of about 1 to 1,000 μm, in the range of about 5 to 500 μm, or in the range of about 10 to 300 μm. The range includes the endpoints.

The specific surface area of the beads is in the range of about 0.1 to 500 m²/g, 10 to 300 m²/g, or 50 to 200 m²/g.

In some embodiments, the bead may be coupled with a cleavable linker. In some embodiments, the cleavable linker is selected from, but not limited to, naturally occurring and synthetic α, β, γ, or δ amino acids.

In some embodiments, the coupling agent is, for example, but not limited to, dicyclohexylcarbodiimide (DCC), diisopropylcarbodiimide (DIC), ethyl-(N′,N′-dimethylamino)propylcarbodiimide hydrochloride (EDC), 3-(diethylphosphoryloxy)-1,2,3-benzotriazin-4(3H)-one (DEPBT), 4-(4,6-dimethoxy-1,3,5-triazin-2-yl)-4-methylmorpholinium chloride (DMTMM), and carbonyldiimidazole (CDI). Preferably, the coupling agent is diisopropylcarbodiimide (DIC).

In some embodiments, the solvent for the coupling step may be selected from, but not limited to, N,N-dimethylformamide, dimethyl sulfoxide, dioxane, and tetrahydrofuran. In some embodiments, the solvent is N,N-dimethylformamide.

In another preferred embodiment, a combinatorial library of compounds comprising a plurality of peptoid molecules each having at least one unit of formula (I):

Wherein:

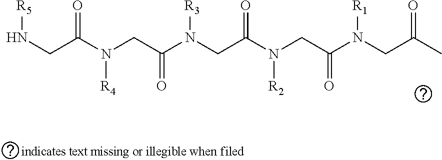

R₁, R₂, R₃, R₄, R₅ independently comprise one or more amines or groups derived from comprising:

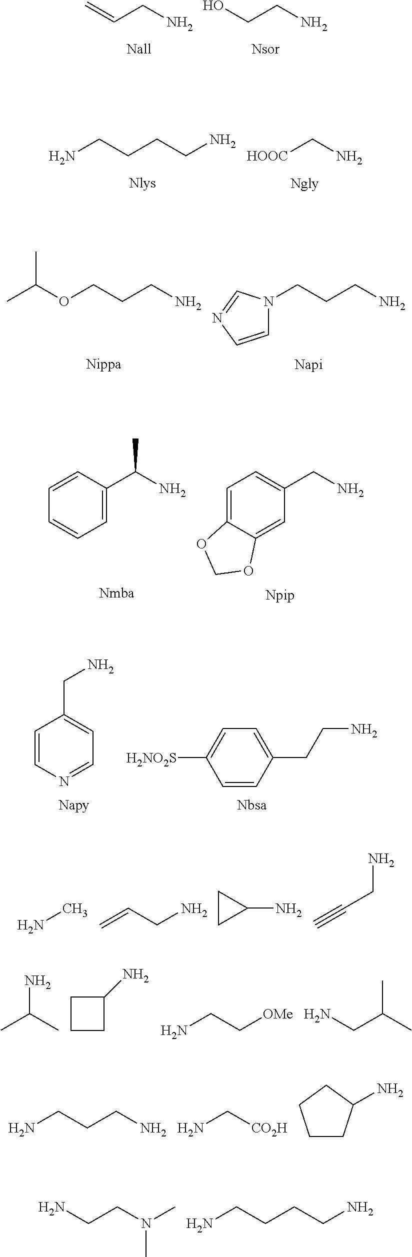

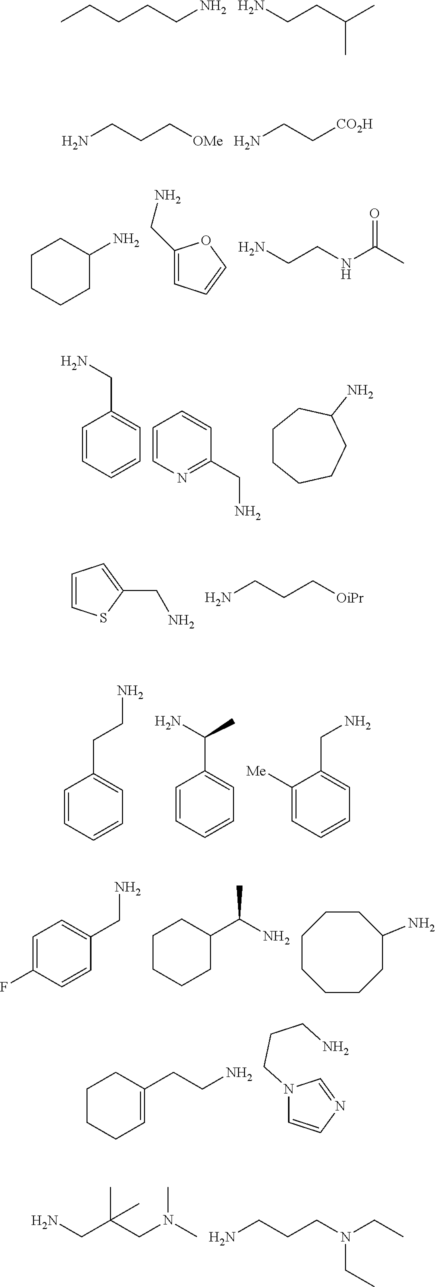

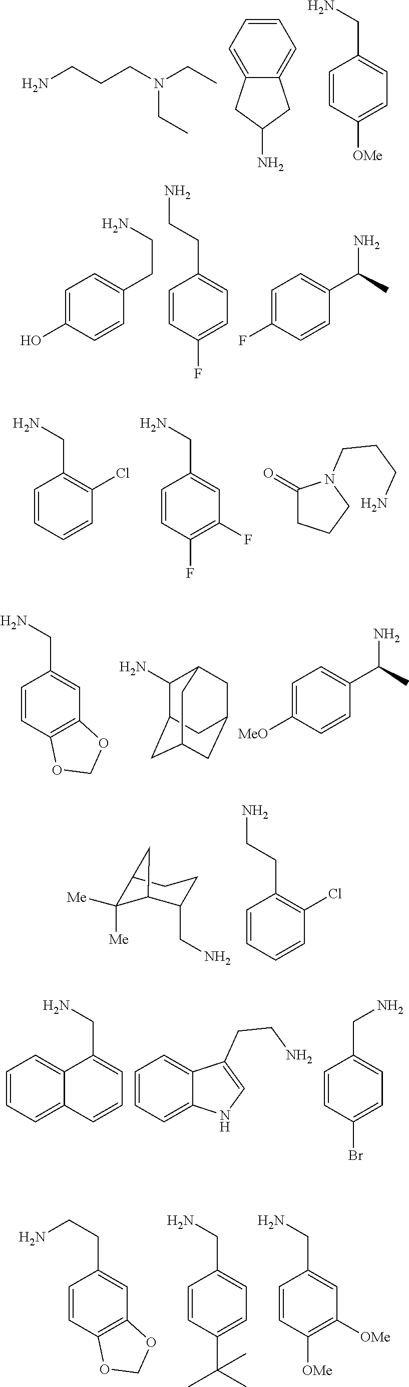

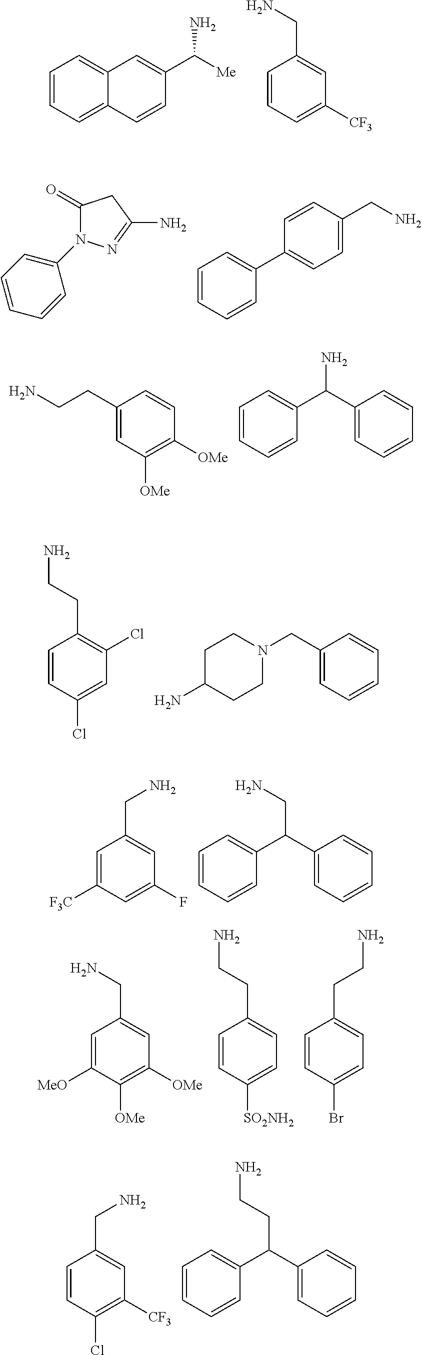

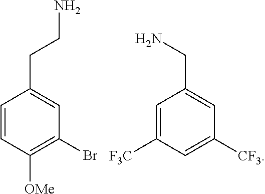

In other embodiments, one or more peptoid ligands are attached to or covalently bound to a polymer. In preferred embodiments, the polymer increases the avidity of the peptoids. The type of polymer is not limited to any one specific type. In some embodiments, a polymer comprises one or more monomers, the one or more monomers comprising one or more ligands, wherein the ligands are peptoids of at least 5-mer having a general structural formula (I):

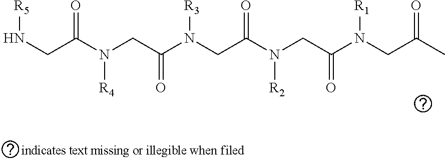

wherein

R₁, R₂, R₃, R₄, R₅ independently comprise one or more groups derived from amines comprising:

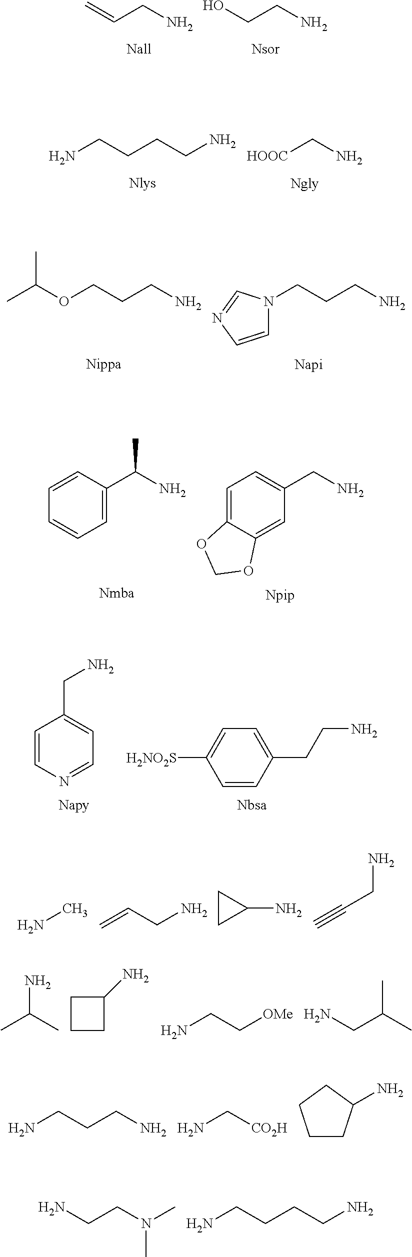

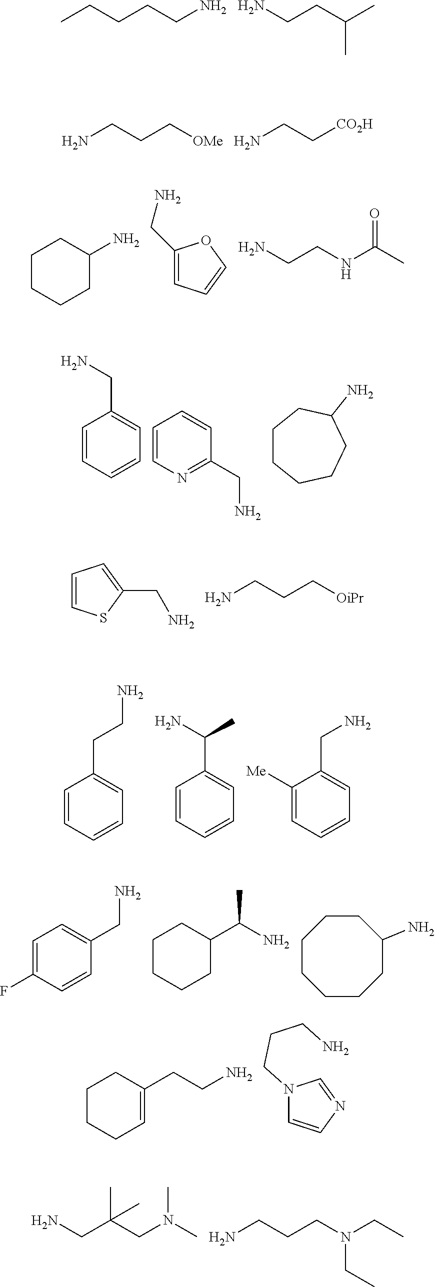

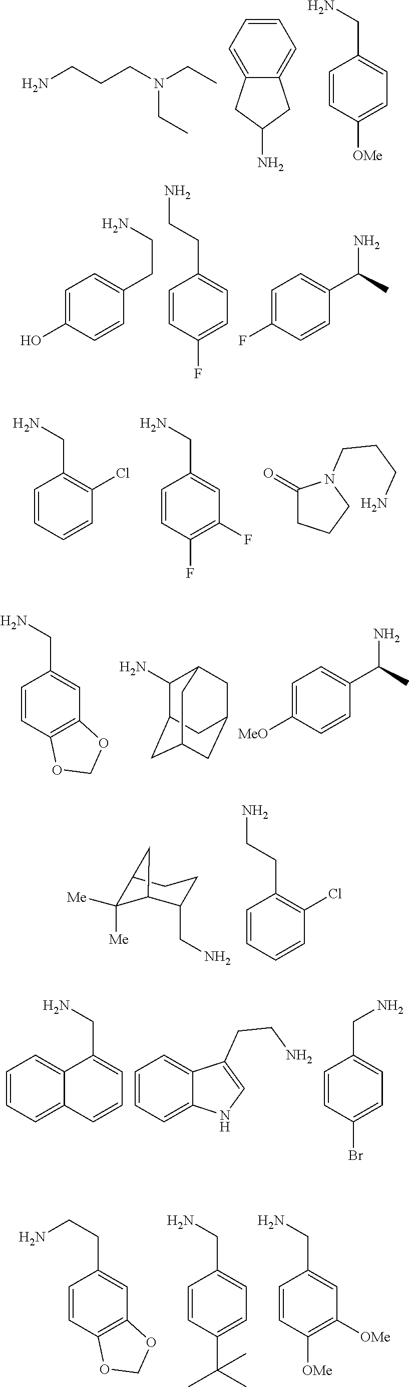

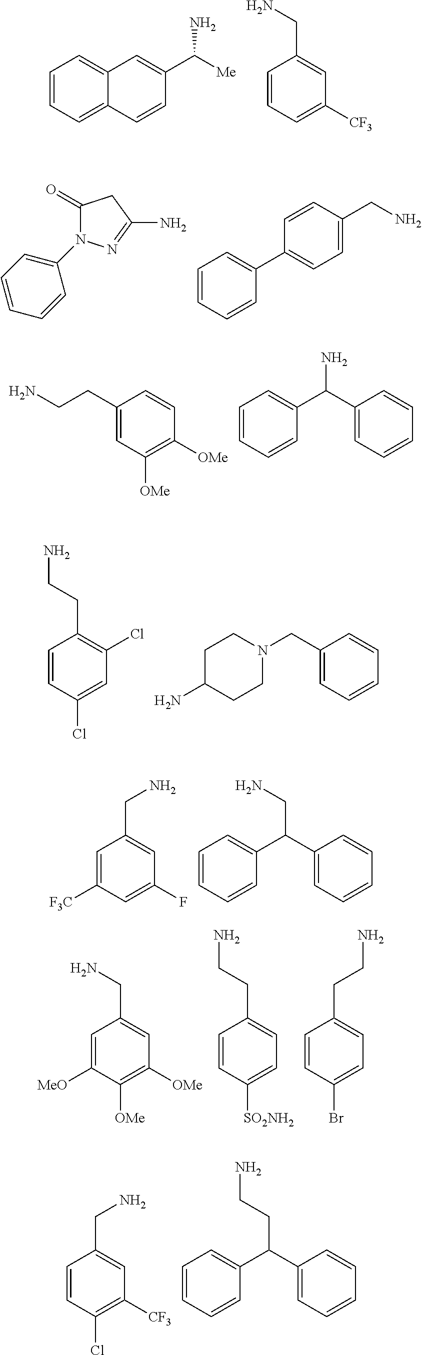

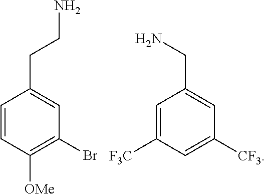

In some embodiments, the monomer comprises: dextran, modified dextran, sugars, amino acids, nucleic acids, synthetic molecules, organic or inorganic molecules, carbohydrates, dendrimers, including polypropylamine dendrimers or pAMAM based dendrimers, polyimine(s), including PEI: poly(ethyleneimine), poly(propyleneimine), polyallylamine, sugar backbone based polymers, including cyclodextrin based polymers, dextran based polymers, Chitosan, etc., silan backbone based polymers, such as PMOXA-PDMS copolymers, etc., poly(alkylene oxide), carboxymethylcellulose, polyvinyl alcohol, N-(2-hydroxypropyl)methacrylamide, polyvinyl pyrrolidone, poly-1,3-dioxolane, poly-1,3,6-trioxane, polypropylene oxide, a copolymer of ethylene and maleic acid anhydride, a polyactide/polyglycolide copolymer, a polyaminoacid, a copolymer of poly(ethylene glycol) and an amino acid, a polypropylene oxide/ethylene oxide copolymer, cross-linking agents, variants or combinations thereof. In other embodiments, the dextran polymer is linear, branched, or combinations thereof. In some embodiments, the dextran further comprises one or more modified dextran molecules.

In some embodiments, two or more peptoids are linked to each other via linker molecules or cross-linking agents. Examples of cross-linkage agents, include, without limitations: a bifunctional disulfide-forming cross-linking agent or a bifunctional thioether-forming cross-linking agent, a long-chain cross-linking agent, with a molecular weight of about 300 to about 5,000 Da, 1,4-di-[3′,2′-pyridyldithio(propion-amido)butane]; α, ω-di-O-pyridyldisulfidyl-poly(ethylene glycol); a vinyl sulfone such as α, ω-divinylsulfone-poly(ethylene glycol); 1,11-bis-maleimidotetraethylene glycol; and α, ω-diiodoacetamide-poly(ethylene glycol). Examples of linker molecules, include, without limitation: alkyl groups, ether, polyether, alkyl amide linker, a peptide linker, a polypeptide linker, a modified peptide or polypeptide linker, a peptide nucleic acid (PNA) a Poly(ethylene glycol) (PEG) linker, a streptavidin-biotin or avidin-biotin linker, polyaminoacids (e.g. polylysine), functionalized PEG, polysaccharides, glycosaminoglycans, dendritic polymers PEG-chelant polymers, oligonucleotide linker, phospholipid derivatives, alkenyl chains, alkynyl chains, disulfide, or a combination thereof.

Diagnostics:

In embodiments, a method of diagnosing an autoimmune disease comprises: contacting a ligand library as embodied herein, with a sample from a patient wherein molecules or biomarkers associated with an autoimmune diseases are specifically bound by one or more ligands. Identification of the ligands can be via any detectable label or secondary molecule which comprises a detectable label. For example, the ligand can be detectably labeled and binding of a specific molecule can be detected by quenching assays, or detection of a label, e.g. radioactive, fluorescence, luminescence and the like. The specificity of the ligands embodied herein, allows a medical practitioner to correctly diagnose the disease as many autoimmune diseases have overlapping symptoms and can be misdiagnosed.

In some embodiments, the ligands are linked to a detectable label (detectable molecule), either directly or linked via a suitable linker. The present invention is not limited to any particular linker group. Indeed, a variety of linker groups are contemplated, suitable linkers could comprise, but are not limited to, alkyl groups, ether, polyether, alkyl amide linker, a peptide linker, a polypeptide linker, a modified peptide or polypeptide linker, a peptide nucleic acid (PNA) a Poly(ethylene glycol) (PEG) linker, a streptavidin-biotin or avidin-biotin linker, polyaminoacids (e.g. polylysine), functionalized PEG, polysaccharides, glycosaminoglycans, dendritic polymers PEG-chelant polymers, oligonucleotide linker, phospholipid derivatives, alkenyl chains, alkynyl chains, disulfide, or a combination thereof.

In another embodiment, the detectable label is linked to the ligand, through a chemical bond, or noncovalently, through ionic, van der Waals, electrostatic, or hydrogen bonds.

In one embodiment, the detectable label is a fluorophore. Fluorophores include any compound, composition or molecule capable of emitting light in response to irradiation. In many instances, fluorophores emit light in the visible region of the spectrum. In other instances, the fluorophores can emit light in the non-visible regions of the spectrum, such as ultraviolet, near-ultraviolet, near-infrared, and infrared. For example and without limitation, examples of fluorophores include: quantum dots; nanoparticles; fluorescent proteins, such as green fluorescent protein and yellow fluorescent protein; heme-based proteins or derivatives thereof; carbocyanine-based chromophores, such as IRDye 800CW, Cy 3, and Cy 5; coumarin-based chromophores, such as (7-diethylamino-3-(4′-maleimidylphenyl)-4-methylcoumarin) (CPM); fluorine-based chromophores, such as fluorescein, fluorescein isothiocyanate (FITC); and numerous ALEXA FLUOR™ chromophores and ALEXA FLUOR™ bioconjugates, which absorb in the visible and near-infrared spectra. The emission from the fluorophores can be detected by any number of methods, including but not limited to, fluorescence spectroscopy, fluorescence microscopy, fluorimeters, fluorescent plate readers, infrared scanner analysis, laser scanning confocal microscopy, automated confocal nanoscanning, laser spectrophotometers, fluorescent-activated cell sorters (FACS), image-based analyzers and fluorescent scanners (e.g., gel/membrane scanners).

In another embodiment, a detectable label is a chemiluminescent moiety. Chemiluminescent moieties include any compound, composition or molecule capable of emitting light in response to a chemical reaction. A bioluminescent compound refers to a naturally occurring form of a chemiluminescent compound. Examples of chemiluminescent compounds include: lucigenin, luminol. Examples of bioluminescent compounds include: luciferins, coelenterazines. The emission from chemiluminescent compounds can be detected by luminometers or scanning spectrometers.

The labeled ligands can be used as diagnostics for both in vivo and in vitro use. For example, a compound may be identified as a ligand for a certain receptor which may be up-regulated in a disease state (e.g. auto-antigens, or receptors for auto-antigens). The compounds can be labeled with a detectable label or secondary molecule which comprises a detectable label in order to detect binding.

In some embodiments, the methods are used to identify and quantify a specific molecule in a sample, for example, for diagnostic purposes, or monitoring the response to treatment or metabolism of drugs in vivo, etc. In some embodiments, a method of quantifying a specific molecule, e.g. a protein in a sample, the method comprises the steps of: placing the sample containing the specific target molecule into a receptacle, contacting the sample with one or more ligands of Formula I wherein the ligands or a secondary molecule (e.g. an antibody) are conjugated to a detectable label and quantifying the target molecule. In some embodiments, a method of quantifying a specific molecule, e.g. a protein in a sample, the method comprises a Förster Resonance Energy Transfer (FRET), Bioluminescence Resonance Energy Transfer (BRET), or fluorescence polarization assay.

In other embodiments, the assay is an immunoassay. For example, ELISA's, competitive ELISA's, RIA's, Western blots, gels, immunoblots, and the like. In other examples, the assays, comprise nucleic acid based assays (e.g. hybridization assays). In embodiments, the assays are high-throughput screening assays.

In embodiments, the target is present in a sample comprising: a liquid, a semi-liquid, a gel, a biological sample, an intact cell, a permeabilized cell, a disrupted cell, a cell homogenate, a membrane, or a cellular organelle.

In one embodiment, a method of diagnosing Neuromyelitis optica (NMO) comprising: contacting a patient sample with a combinatorial library of peptoids, and detecting antibodies specific for aquaporin 4 (AQP4). In preferred embodiments, the peptoids specifically bind to the AQP4 antibodies.

In one embodiment, peptoids which specifically bind to or capture AQP4 antibodies, comprise:

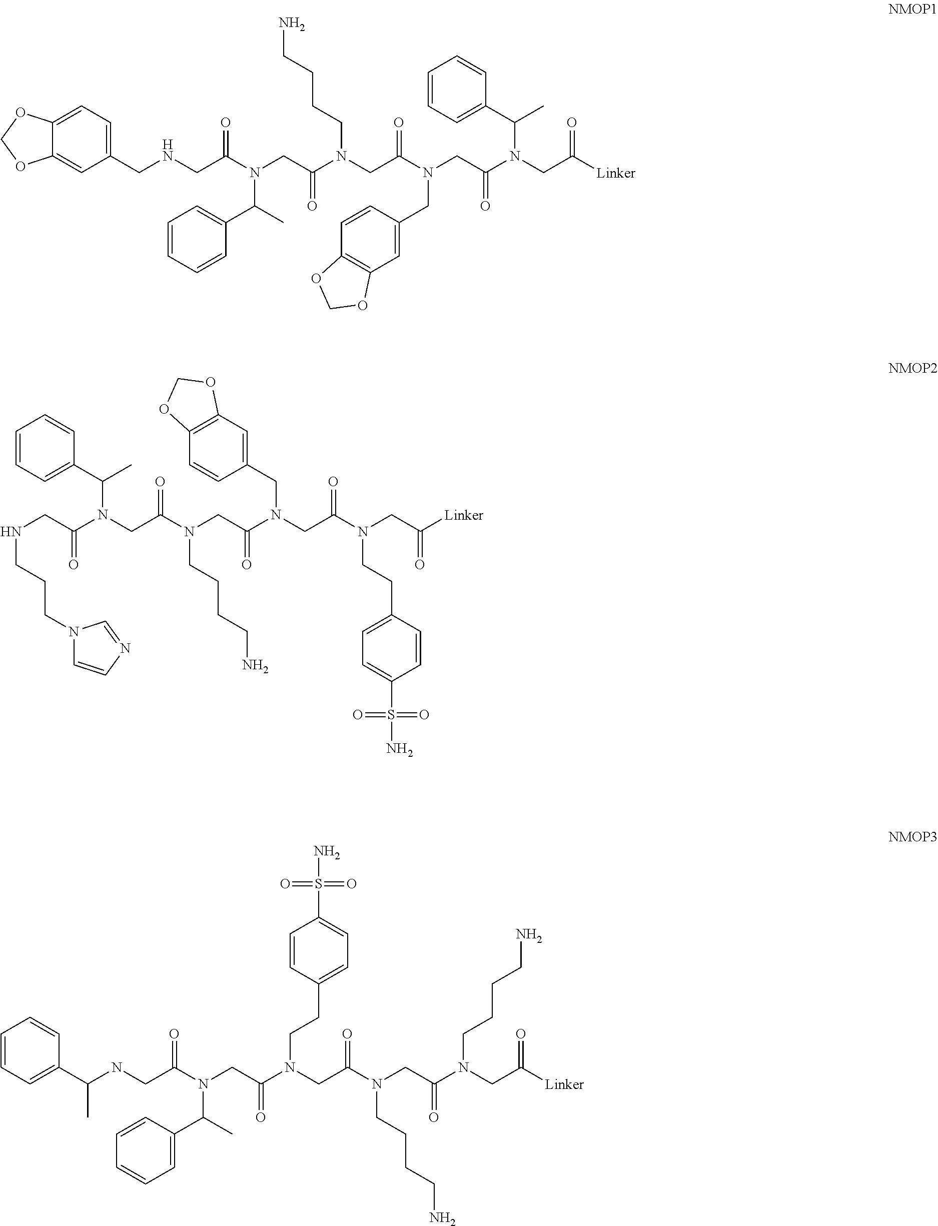

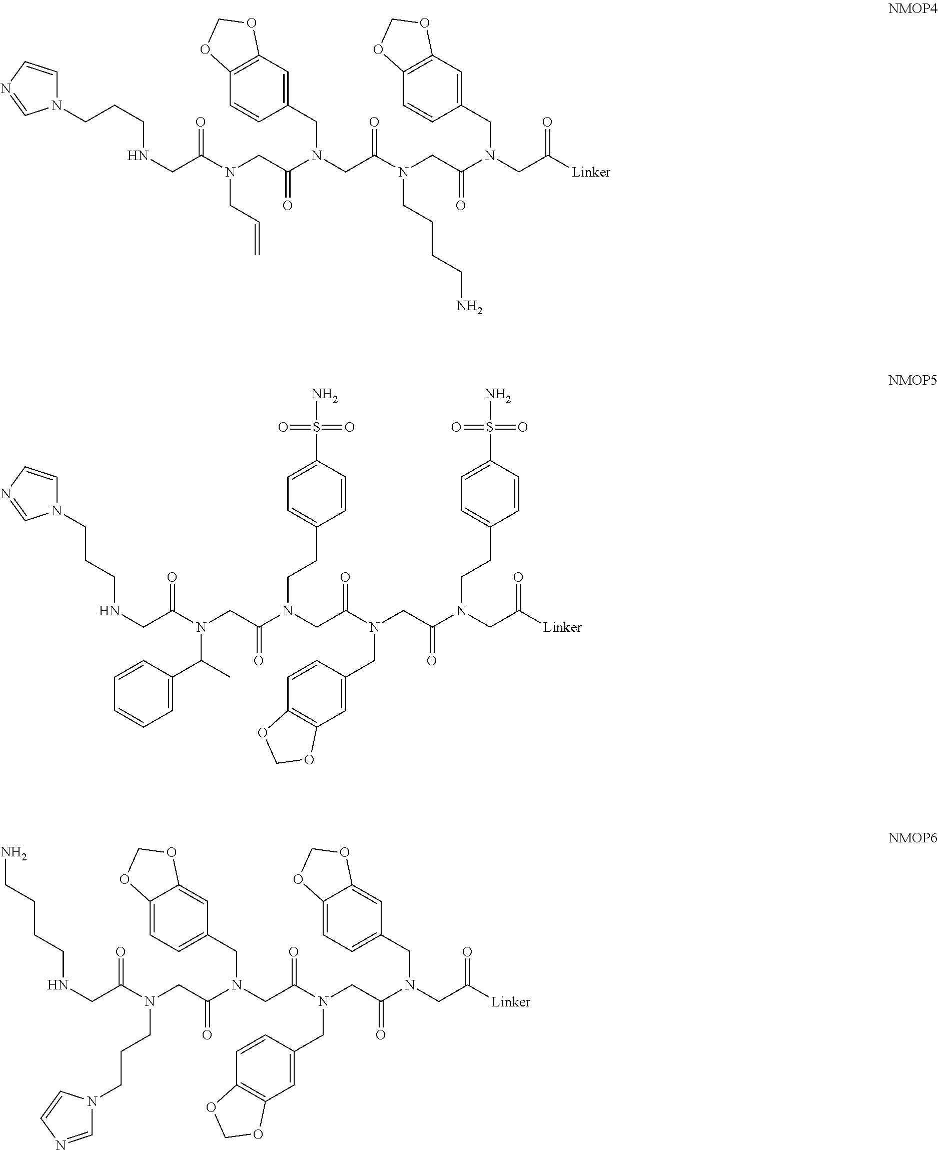

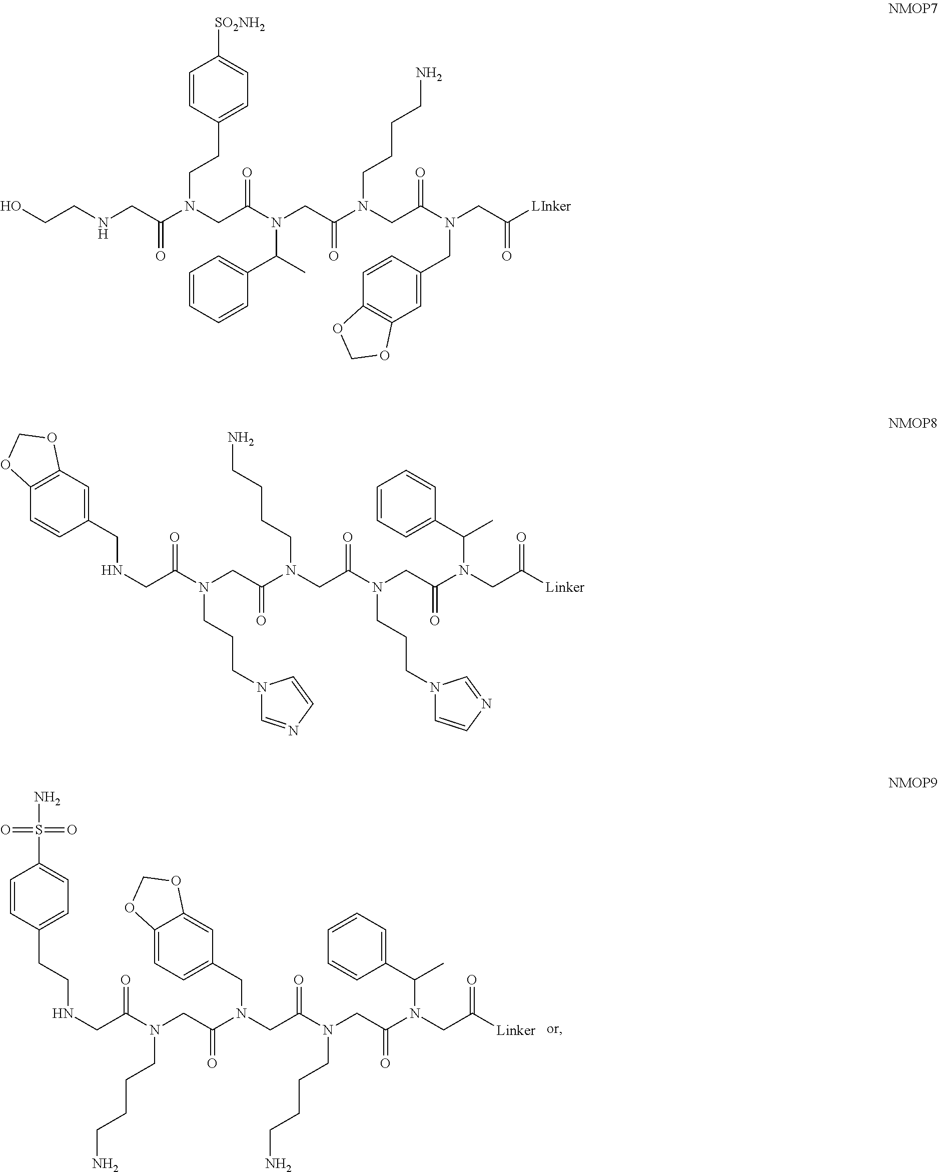

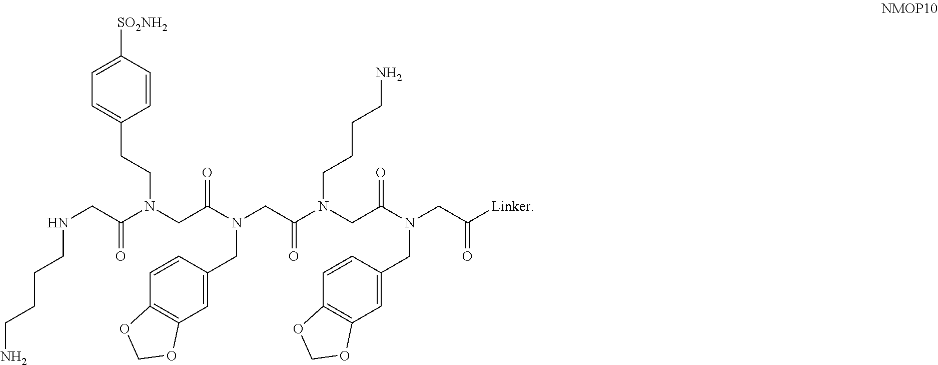

In other embodiments, a method of diagnosing an autoimmune disease comprises obtaining a sample from a patient, screening the sample against a peptoid combinatorial library and identify peptoids which specifically bind to one or more molecules associated with a particular disease. The molecules can be identified as described in detail in the Examples section which follows.

In other embodiments, a method of diagnosing a specific disease or disorder (e.g. cancer) comprises obtaining a sample from a patient, screening the sample against a peptoid combinatorial library and identify peptoids which specifically bind to one or more molecules associated with a particular disease, for example, biomarkers, ligands, etc. The molecules can be identified as described in detail in the Examples section which follows.

Candidate Therapeutic Agent Screening:

In other embodiments, the combinatorial library can be used for screening of candidate therapeutic agents. The drugs can be identified, in one embodiment, on their ability to specifically bind to the peptoids. These can then be tested against biological samples of patients to identify those agents which bind to molecules associated with a particular disease, e.g. AQP4 antibodies, biomarkers, inflammatory molecules and the like.

In one embodiment, a method of treating an autoimmune disease comprises administering to a patient in need thereof, of a therapeutically effective ligand which specifically binds to a molecule associated with an autoimmune disease, e.g. an antibody to an auto-antigen.

In preferred embodiments, the autoimmune disease is Neuromyeltits optica.

The invention has been described in detail with reference to preferred embodiments thereof. However, it will be appreciated that those skilled in the art, upon consideration of this disclosure, may make modifications and improvements within the spirit and scope of the invention.

All documents mentioned herein are incorporated herein by reference. All publications and patent documents cited in this application are incorporated by reference for all purposes to the same extent as if each individual publication or patent document were so individually denoted. By their citation of various references in this document, Applicants do not admit any particular reference is “prior art” to their invention.

### EXAMPLES

The following non-limiting examples are illustrative of the invention.

### Example 1

### Discovery of Peptoid Ligands for Anti-Aquaporin 4 Antibodies

Materials and Methods

Materials:

All chemicals and solvents were purchased from commercial suppliers and used without further purification. HPLC grade solvents were used. Unless otherwise stated, all reactions were carried out at room temperature.

Serum samples from NMO patients (anti-AQP4 Ab+ or Ab−), relapsing-remitting MS patients and normal controls were obtained from the Accelerated Cure Project Repository. The presence of Anti-AQP4 antibodies in these sera was verified by a cytotoxicity assay, in which sera were incubated with AQP4-transfected or control non-transfected 293 cells and with human complement, and cell death was determined by measuring release of intracellular proteases as compared to Triton-X-induced complete lysis. Human monoclonal anti-AQP4 antibody rAb-53 was derived from a clonally-expanded cerebrospinal fluid plasmablast isolated from an NMO patient (Bennett et al., 2009, Ann Neurol 66, 617-629). The control antibody used in this study was an antibody unrelated to NMO, rAb-2B4, which is a human IgG to the nucleocapsid protein of measles virus.

Narcolepsy sera samples were identified as Controls (NC), Narcolepsy (NP) and Narcolepsy with psychotic features (NS).

Peptoid Combinatorial Library Synthesis: The peptoid library was synthesized on hydrophilic TentaGel MB NH2 beads (160 μm, Rapp Polymere) following a microwave assisted solid-phase synthesis protocol (Olivos et al., 2002, Organic Letters 4, 4057-4059). The Tentagel beads (1 g, ˜500,000 beads, 0.52 mmol/g) were swelled in DMF for 2 hours before use. DMF was used as solvent unless otherwise mentioned. Fmoc-Met-OH (0.77 g, 2.08 mmol) was coupled on the beads using HBTU (0.77 g, 2.08 mmol), DIPEA (0.45 ml, 2.6 mmol). Fmoc was deprotected by 20% piperidine. Beads were washed thoroughly with DMF after each step. 2 mL 2M DIC solution and 2 mL 2M 2-Bromoacetic acid solution were added together for pre-activation for 5 min. The 4 mL combined solution was then added to one portion of beads and gently shook till completion. The reaction was monitored by chloranil test; a clear negative result after 5 min indicates the amine was acylated by the corresponding bromoacid. 2M solution of the amine was used with an incubation time of 2 hour at RT. After methionine (which helps CNBr cleavage of the compound from the bead), three amines (furfuryl amine, diaminobutane, and diaminobutane) were added sequentially and this part served as the constant linker region for the compounds in the library.

The 5 variable positions in the compounds were synthesized using 10 amines by split and pool method giving a total diversity of 10⁵ compounds. The acid labile protecting groups were removed by cleavage cocktail (95% TFA, 2.5% water and 2.5% triisoproylsilane) for 2 hour at RT. The beads were then washed extensively with DCM.

Serum Screening Protocol Using a One Bead One Compound Peptoid Library:

The beads were swelled in DMF overnight, then washed several times with water and kept in water with gentle shaking for 12-15 hour followed by equilibrating in TBST for at least 5 hours before screening.

To pre-screen the library, serum samples from healthy people were used as the control serum to pre-screen the library to eliminate the beads binding to the antibodies present in the healthy serum samples. Six different normal control sera (NC) were pooled in TBST. Enough buffer was added to achieve a final total protein concentration of 100 μg/mL The beads (200 mg, pre-processed as described above) were incubated with 4 mL of NC serum sample (100 μg/mL) at 4° C. overnight with gentle shaking. The beads were washed three times with TBST and incubated with anti-human secondary antibody conjugated with quantum dot (Qdot 655, Invitrogen; 20 μL in 4 mL TBST) at RT for 2 hours. The beads were washed again three times with TBST and the red beads were removed under a fluorescent microscope to get the denuded library. The remaining beads were washed several times with TBST to screen against the disease serum.

The pre-screened library was incubated with a pool of sera obtained from 6 NMO patients with high levels of anti-AQP4 Ab. The total protein concentration was adjusted to 50 μg/mL total protein in 50% PBS Starting Block Buffer (Thermo Scientific). The beads were incubated at 4° C. overnight by gentle shaking. After the incubation step the beads were washed three times with TBST and treated with quantum dot-conjugated antihuman secondary antibody (Qdot 655; 20 μL, in 4 mL TBST) at RT for 2 hour. The beads were washed again with TBST three times and the beads with a red halo were collected under a fluorescent microscope.

The hit beads were stripped off the antibody by heating in a 1% SDS solution at 95° C. for 15 minutes and washed extensively with water before validating by rebinding experiments using the same procedure described above

Identification of the Compound:

The antibody bound to the hit beads were stripped off by using 1% SDS solution at 95° C. and the beads were washed extensively with water followed by a 50% acetonitrile-water mixture. The beads were treated with CNBr (30 mg CNBr in 1 mL cleaving cocktail solution, ACN:H₂O:HOAc in the ratio 5:4:1) to cleave the compound from the bead. The structure of the compounds was then determined by tandem MALDI TOF-TOF mass spectrometry.

Antibody Depletion Experiment:

Purified AQP4 protein was coupled to an amine-reactive protein immobilization column (MicroLink Protein Coupling Kit, Thermo Scientific) following the manufacturer's protocol. The protein coupling efficiency was found to be greater than 95%.

Approximately 80-120 μg of protein was immobilized per column. Serum samples were incubated with the AQP4-immobilized column overnight at 4° C. to deplete the anti-AQP4 specific antibodies from the serum. The experiment was repeated twice with the same serum samples to ensure a complete depletion. Similarly BSA immobilized column was used as a control column in this experiment. These depleted serum samples were used in microarray in appropriate dilution.

Preparation of Maleimide Slides:

25×75 mm glass slides (Sigma-Aldrich) were cleaned in piranha solution (sulfuric acid/hydrogen peroxide, 7:3). Epoxy functionality was incorporated onto the slides by salinization using a solution of 2% 3-glyicidoxypropyl-trimethoxisilane in 16 mM acetic acid and 95% ethanol. After 1-2 hour incubation, the slides were washed with ethanol and cured at 150° C. for at least 2 hour. The resulting amine slides were incubated in a solution of 400 mL of PEG (polyethylene glycol) and 1.5 mL H₂SO₄ for 2 hour. The slides were rinsed with water and spin-dried. The PEG-derivatized surface was reacted with a solution of 100 mM PMPI (p-Maleimidophenyl Isocyanate) in DMF for 2 hours, washed with ethanol, spin-dried and stored at 4° C.

Microarray Spotting, Hybridization, and Data Analysis:

A stock solution (500 μM) of the peptoid was prepared in 50% DMSO with 50% PBS, and distributed in 384-well plates. All peptoids were shown to be completely soluble in this spotting solution. Slides were spotted on a NanoPrint LM 60 (TeleChem International Inc., Sunnyvale, Calif.) with MP946 Micro Spotting Pins. Spots generated were approximately 120-μm in diameter and were printed with a spot-to-spot spacing of 375 μm. The pins were rinsed in between samples using two cycles of wash (for 10 s) and sonication (for 10 s) in reservoirs containing 10% ethanol followed by drying under reduced pressure (for 10 s). The slides were allowed to stand for at least 2 hours on the printer platform and stored at 4° C. until use. Before incubation with the serum sample, the slides were quenched with 100 mM cysteine in PBS (pH 7.2) for 20 min and washed with deionized water.

The serum sample was diluted in a binding buffer (100 mM phosphate buffer pH 7.2, 150 mM NaCl, 10 mM EDTA) volume of 50 μL containing 0.25% BSA, 0.01% TWEEN® 20. The serum sample (4˜10 μg/mL in total protein concentration, 50 μL) was applied to the array and this was incubated at room temperature for two hours, washed with 1×TBST (3×10 min), then with deionized water three times and dried by centrifugation. Secondary antibody solution (Alexa Fluor® 647 Goat Anti-Human IgG (H+L), Invitrogen, 1:1500) in TBST with 0.25% BSA, was then applied and the array was incubated at room temperature for 1 hour, washed with 1×TBST (3×10 min), then deionized water three times and dried by centrifugation.

Slides were scanned on a microarray scanner (GenePix 4200AL, Molecular Devices, USA) by using the 488/635 nm laser at 100% power and a 500-photomultipliertube gain. All the scanned images were analyzed using GenePix Pro 6.0 (Molecular Devices, USA) software. The experiments were done in triplicate, and each group of three included slides printed in different batches to avoid bias due to batch-to-batch differences in the slides. Local background subtracted mean (F635 Mean-B635) spot intensities were used for further analysis. These signal intensities were used for downstream analysis using Excel software. The same criteria were used to analyze all the test experiments on microarray.

Analysis of Blind Human Serum Samples:

The blind human serum samples were analyzed in exactly the same way as described above. Local background subtracted mean (F635 Mean-B635) spot intensities were used as net intensities. For the heat map, net intensities was subtracted by overall F635 Median, and analyzed by TreeView (rana.lbl.gov/EisenSoftware.htm) software.

Results

Screening Bead-Displayed Peptoid Libraries for Antibody Ligands:

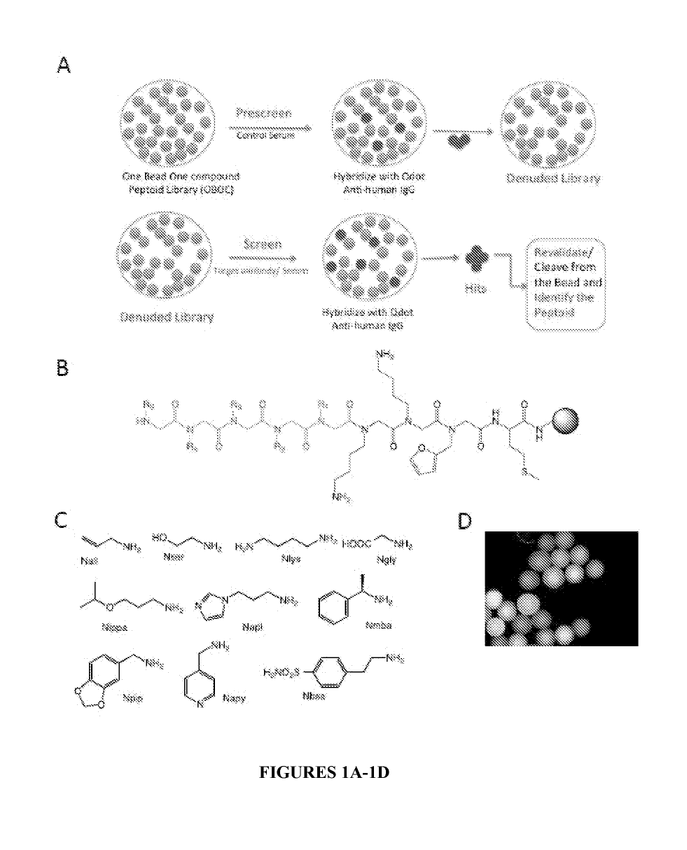

Previous data (Reddy et al., 2011, Cell 144, 132-142) employed comparative screening of several thousand peptoids arrayed on a modified glass slide against case and control serum samples. In order to substantially increase the number of compounds that could be employed in such a screen, a protocol was developed that allowed one bead one compound (OBOC) libraries synthesized on hydrophilic TentaGel beads to be employed directly in the screening step. Libraries of hundreds of thousands or even millions of peptoids are easily prepared in this format by split and pool solid phase synthesis (Alluri et al., 2003 J Amer Chem Soc 125, 13995-14004; Figliozzi et al., 1996 Methods Enzymol 267, 437-447; Lam et al., 1991 Nature 354, 82-84) and these libraries can be employed productively in screening experiments using recombinant proteins or cells as targets. The strategy that we envisioned is shown in FIG. 1A. The bead library would first be incubated with a pool of control serum samples followed, after washing, by a red quantum dot-labeled secondary antibody to “light up” beads that retain significant amounts of these uninteresting antibodies or bind directly to the secondary antibody.

After removal of these beads, the denuded library would then be exposed to a pool of NMO serum samples and the labeled secondary antibody. Hits from this screen would be collected as possible ligands for NMO-specific antibodies and analyzed further.

A library of peptoids containing five variable positions after an invariant linker of four residues was constructed using the sub-monomer synthetic method (Zuckermann et al., J Amer Chem Soc 114, 10646-10647, 1992). Ten amines were used in the synthesis of the library (FIG. 1C), providing a theoretical diversity of 100,000 compounds. The linker (FIG. 1B) contained a C-terminal methionine residue to facilitate cyanogen bromide-mediated release of the compound from the bead after screening, a furan-containing residue to facilitate post-screening labeling of the compound and two lysine-like peptoid residues (Nlys), which are charged at neutral pH and should aid in the presentation of the peptoid in aqueous solution.

As described above, the library (≈100,000 beads) was first exposed to a pool of six serum samples obtained from control individuals that do not have NMO, followed by fluorescently labeled anti-human IgG secondary antibody. Beads that exhibited an obvious fluorescent halo under a low power fluorescence microscope were removed using a micropipette. The remainder of the library was washed several times with buffer and then incubated with a pool of six serum samples obtained from NMO patients whose serum tested positive for complement-mediated cytotoxicity using AQP4-transfected HEK293 cells, followed by labeled secondary antibody. Beads that displayed above background binding of antibodies as evidenced by the red halo (FIG. 1D) were picked.

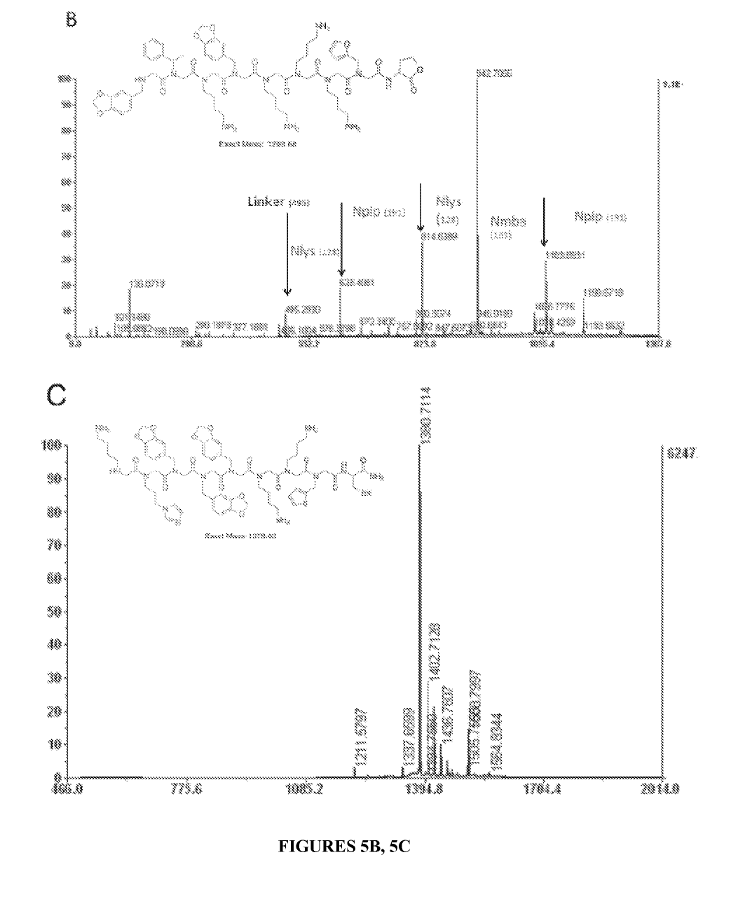

To verify that the beads visualized at this stage are indeed candidate hits, the binding experiment was redone. After stripping the beads with 1% SDS, washing extensively to remove the SDS and re-equilibrating the beads, the NMO serum pool was re-applied. After this step, a total of 43 beads were deemed potential hits. These were segregated into the wells of a microtiter plate. The peptoids were released into solution by cleavage with CNBr and sequenced by tandem mass spectrometry (FIGS. 5A-5D).

Initial Characterization of Screening Hits:

Due to a variety of complexities with bead-based screening technology, it is often the case that compounds that appear to be hits at the bead stage fail to validate in subsequent assays (Chen et al., Journal of combinatorial chemistry 11, 604-611, 2009). Moreover, in this screen, it is possible that one of the pooled NMO samples may have had very high levels of an antibody idiosyncratic to that patient and peptoid ligands of poor diagnostic utility might be isolated. Therefore, it is imperative to assess the ability of the peptoid hits to distinguish several individual case and control serum samples on a different analytical platform. Ten (Table 1) of the 43 hits that corresponded to the brightest beads at the screening stage were resynthesized and spotted onto chemically modified glass slides (Lesaicherre et al., 2002, Bioorg Med Chem Letters 12, 2079-2083; Reddy and Kodadek, 2005, Proc Natl Acad Sci USA 102, 12672-12677). A fluorescein-labeled derivative of one of these peptoids NMOP6 (NMO Peptoid 6) was synthesized, and was employed as a control to ensure that the spotting process proceeded efficiently. A peptoid that was found to bind directly to the secondary antibody, NMOP8, was also spotted onto the array as an internal control.

A derivative of dinitrophenol (DNP), a small molecule that is recognized by antibodies present in most people, was also spotted as another control.

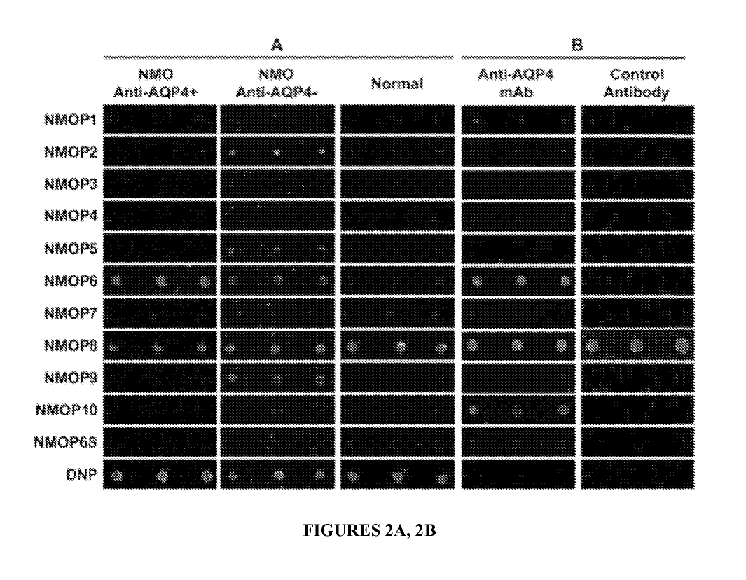

The slides were then incubated with individual serum samples (see methods) and, after washing, fluorescently labeled secondary antibody. As shown in FIG. 2A, when the array was exposed to serum from a control patient, not suffering from NMO, there was little signal observed on any of the arrayed peptoids, except, of course, NMOP8. DNP, as expected, also registered a strong signal. A strong signal was observed in the fluorescein channel for the labeled peptoid, confirming that spotting had proceeded efficiently. When the experiment was repeated with a serum sample obtained from an NMO patient whose serum tested positive in an assay that scores the ability of serum antibodies to drive complement-mediated killing that express recombinant AQP4 (Phuan et al., Journal Biol. Chem. 287, 13829-13839, 2012), indicating the presence of anti-AQP4 antibodies, strong signals were observed on NMOP6. When the experiment was conducted with a sample from an NMO patient that tested negative for anti-AQP4 antibodies by the cell-killing assay, significant intensities were observed on NMOP6, NMOP2, NMOP5 and NMOP9. Note that a negative result in the cell-killing assay does not mean that the serum does not contain anti-AQP4 antibodies. They could be present at a level that is insufficient to trigger efficient cell killing in this assay or they could be variants that do not readily fix complement.

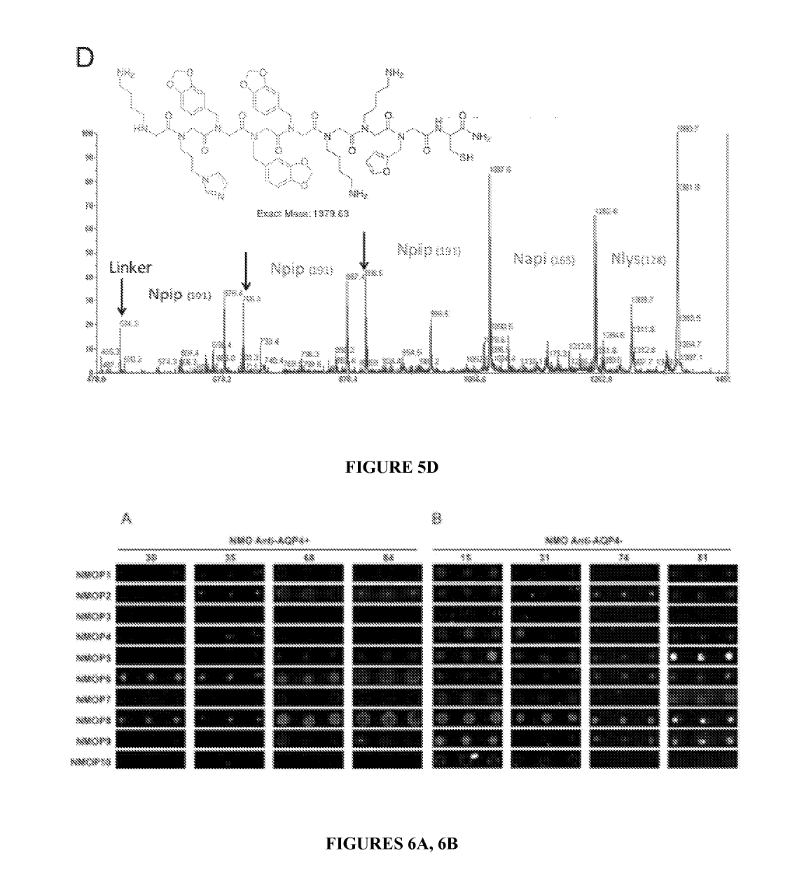

Nonetheless, since the intensity of the NMOP6 signal was markedly lower than was the case for the serum sample that was clearly positive in this assay, it is possible that this peptoid may be a ligand for anti-AQP4 antibodies. The data for the analysis of an additional six individual NMO serum samples, half of which tested positive in the cell-killing assay and half of which did not, are shown in FIGS. 6A, 6B. NMOP6 showed strong signals for the NMO sera that tested positive for cell killing and lower signals for two of the four NMO samples that tested negative for cell killing. NMOP4, NMOP5 and NMOP9 also retained significant amounts of antibodies from certain NMO samples, but were dark on others. The chemical structures of these microarray-validated hits are shown in FIG. 2C. NMOP1, NMOP3, NMOP7 and NMOP10 failed to provide robust signals in any of the samples. The structures of all of these peptoids are shown in Table 1.

TABLE 1

| NAME | STRUCTURE |
|---|---|
| NMOP1 | 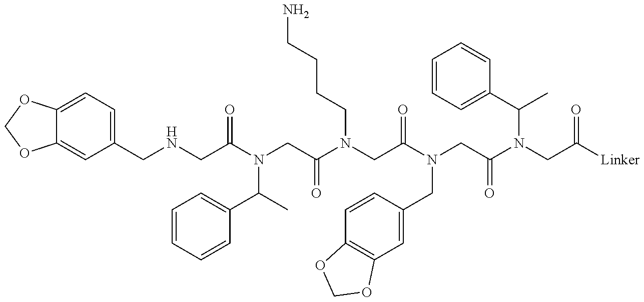 |
| NMOP2 | 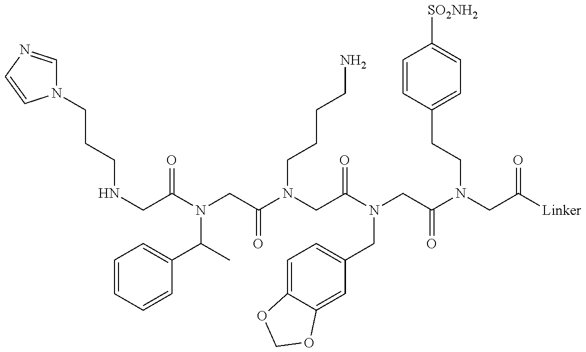 |
| NMOP3 | 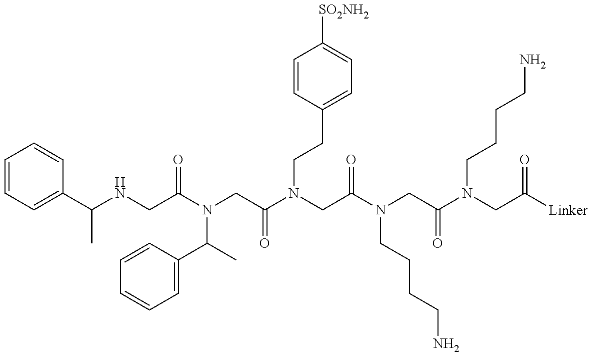 |
| NMOP4 | 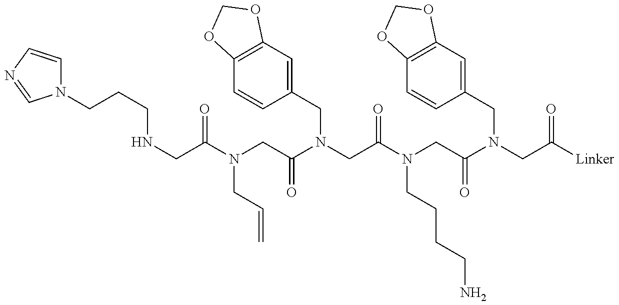 |
| NMOP5 | 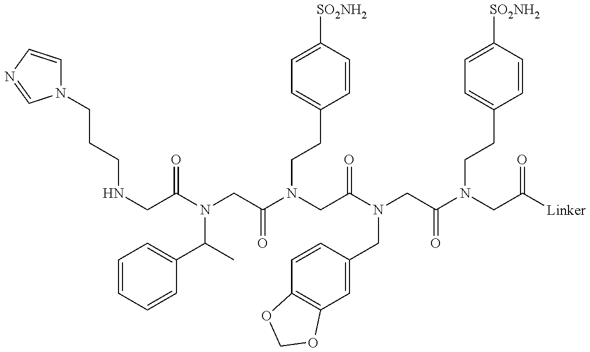 |
| NMOP6 | 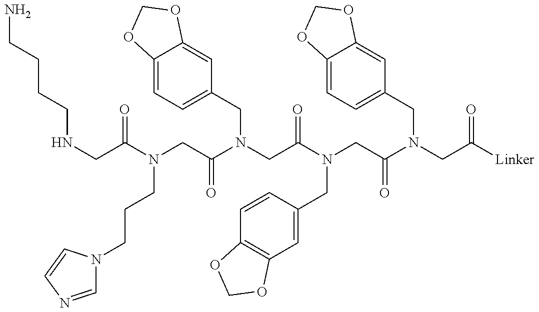 |
| NMOP7 | 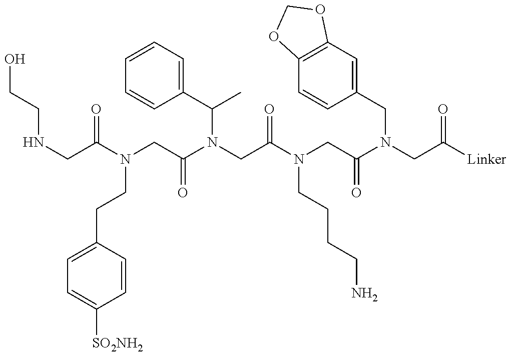 |
| NMOP8 | 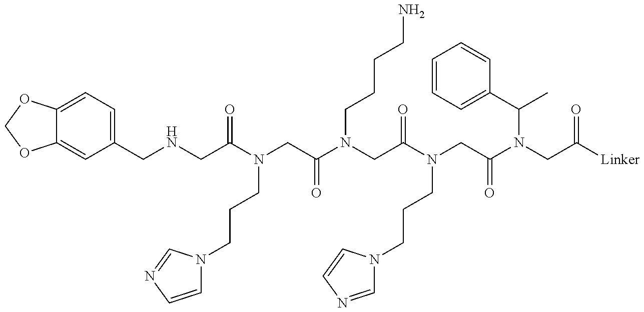 |
| NMOP9 | 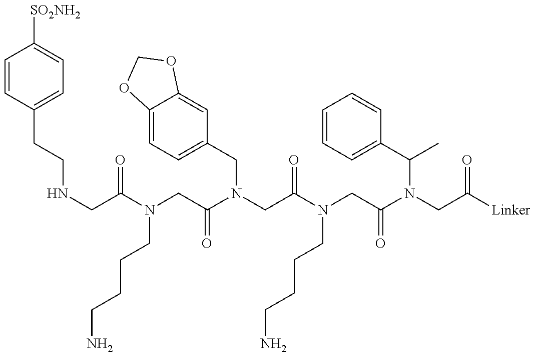 |
| NMOP10 | 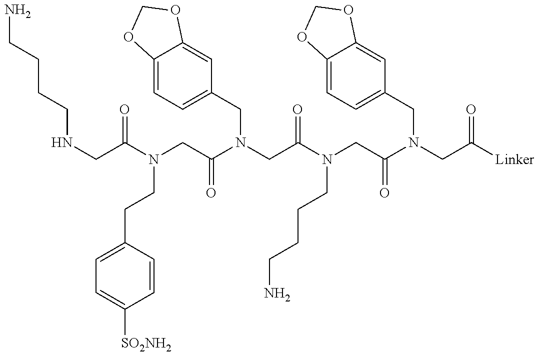 |
| Linker | 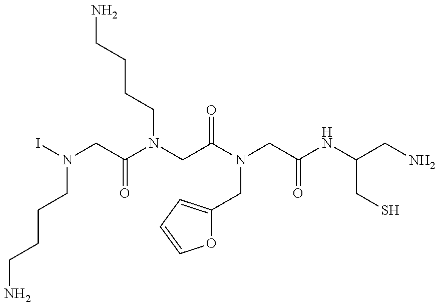 [SMILES: NCCCCN(I)CC(=O)N(CCCCN)CC(=O)N(CC(=O)NC(CN)CS)Cc1ccco1] |
| NMOP6s | 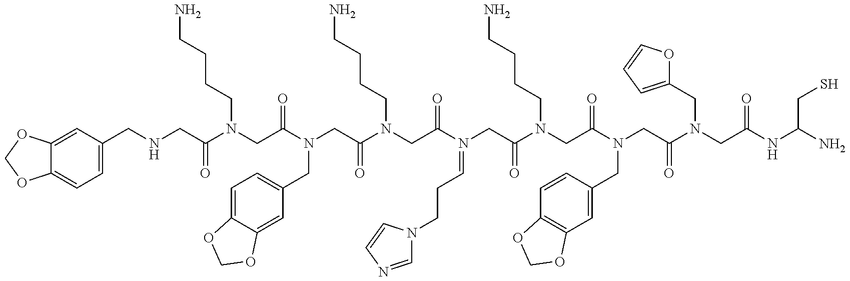 |

TABLE 2
Absolute fluorescence intensity obtained for each peptoid hit on the blind sample analysis

| Serum | Absolute Fluorescence Intensity | | | | | | | | | |
|---|---|---|---|---|---|---|---|---|---|---|
| ID | NMOP1 | NMOP2 | NMOP3 | NMOP4 | NMOP5 | NMOP6 | NMOP7 | NMOP8 | NMOP9 | NMOP10 |
| 1 | 81.67 | 57.67 | 91.67 | 74.67 | 80.00 | 1844.33 | 106.33 | 1856.00 | 81.00 | 57.00 |
| 2 | 94.83 | 74.94 | 103.14 | 82.14 | 94.43 | 1259.45 | 151.51 | 1861.44 | 1012.67 | 162.69 |
| 3 | 98.75 | 85.88 | 102.32 | 80.32 | 110.43 | 88.03 | 115.74 | 1870.14 | 106.13 | 94.56 |
| 4 | 124.79 | 89.36 | 104.38 | 107.43 | 105.74 | 55.40 | 138.60 | 1857.15 | 107.08 | 71.90 |
| 5 | 123.63 | 601.62 | 114.81 | 97.25 | 740.43 | 1848.11 | 145.25 | 1894.38 | 922.50 | 80.08 |
| 6 | 123.37 | 88.84 | 130.75 | 90.23 | 85.54 | 68.22 | 152.17 | 1891.20 | 129.70 | 67.07 |
| 7 | 113.23 | 1067.83 | 112.69 | 76.66 | 95.85 | 1885.37 | 114.09 | 1904.22 | 129.66 | 75.67 |
| 8 | 93.89 | 80.12 | 95.68 | 89.88 | 105.02 | 1863.10 | 125.22 | 1900.09 | 112.63 | 89.81 |
| 9 | 90.58 | 1097.41 | 108.23 | 84.86 | 122.02 | 1857.46 | 120.54 | 1858.64 | 81.50 | 100.32 |
| 10 | 94.72 | 65.56 | 110.09 | 102.05 | 775.81 | 82.28 | 143.16 | 1888.82 | 97.43 | 67.35 |
| 11 | 104.56 | 90.87 | 117.74 | 87.94 | 80.01 | 671.77 | 144.98 | 1898.94 | 991.69 | 289.93 |
| 12 | 97.34 | 87.10 | 118.75 | 116.05 | 82.74 | 53.72 | 128.52 | 1877.65 | 104.02 | 72.34 |
| 13 | 95.65 | 876.78 | 92.21 | 94.94 | 732.19 | 947.34 | 134.17 | 1863.39 | 97.83 | 63.37 |
| 14 | 88.46 | 81.36 | 120.11 | 104.50 | 81.51 | 78.14 | 143.43 | 1904.52 | 81.99 | 76.95 |
| 15 | 127.01 | 487.20 | 124.61 | 493.24 | 80.64 | 272.42 | 152.39 | 1898.43 | 91.90 | 101.40 |

Peptoid NMOP6 is a Ligand for Anti-AQP4 Antibodies:

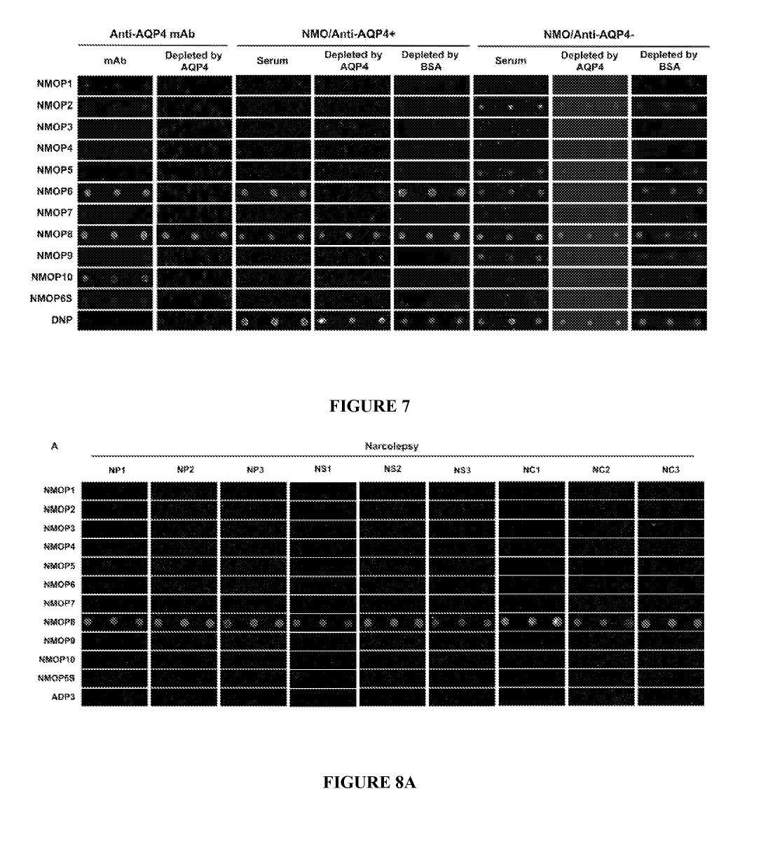

To test the hypothesis that NMOP6 is a ligand for anti-AQP4 antibodies, the array was exposed to a monoclonal antibody isolated from a patient that binds the full-length M1 isoform of human AQP4 (Bennett et al., 2009, Ann Neurol 66, 617-629) or to a control antibody, called rAb-2B4 that binds the measles virus nucleocapsid and is of the same isotype (IgG₁) as the anti-AQP4 antibody. As shown in FIG. 2B, NMOP6 retained the anti-AQP4 antibody efficiently, but not the control antibody. This appears to be a specific interaction because a scrambled version of NMOP6 (NMOP6s, see Table 1) was also spotted onto the array. This peptoid has the same chemical functionality as NMOP6, but the order of the side chains is scrambled. NMOP6S did not bind the anti-AQP4 monoclonal antibody, or antibodies from the serum of NMO patients (FIGS. 2A-2C and FIG. 7).

NMOP10 also showed a weak signal for the anti-AQP4 Ab, indicating that it is likely a very low affinity ligand for this antibody, but since this peptoid was not of utility in serum screening, this issue was not pursued further.

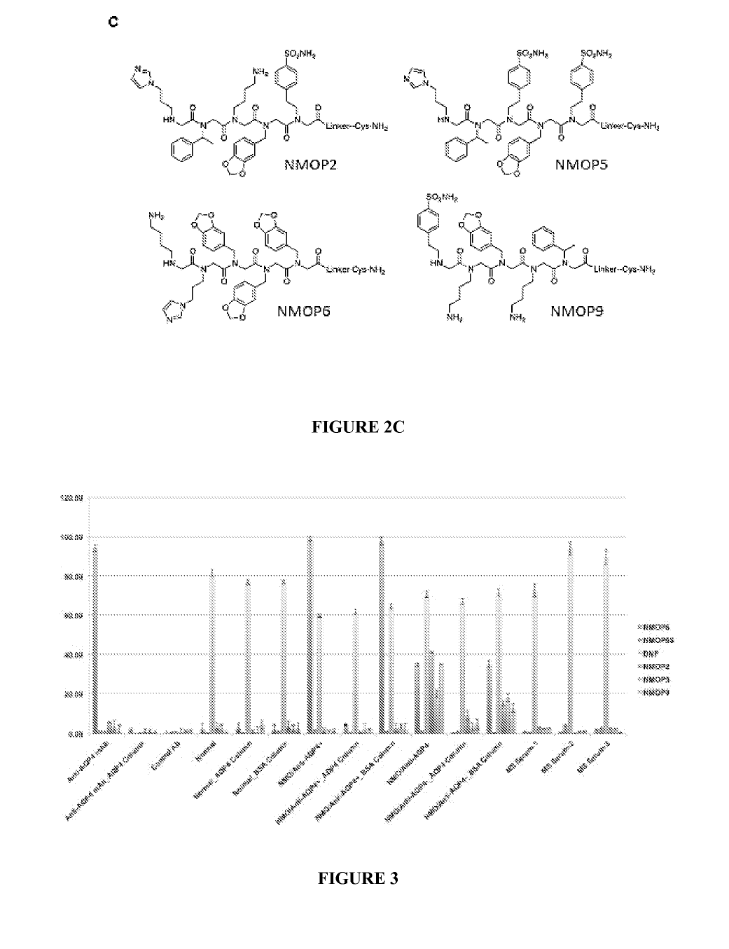

To ask in a different way if NMOP6 indeed recognizes anti-AQP4 antibodies, the serum from an NMO patient was passed over a column of immobilized recombinant AQP4 or, as a control, bovine serum albumin (BSA). The depleted serum was then applied to the array. As shown in FIG. 3, the robust signal observed on NMOP6 for the NMO sample was abolished when the anti-AQP4 antibodies were removed from it, but the signal was unaffected by passage over a BSA column. Moreover, the signal on the DNP control spot was unaffected by passage of the serum over either column, proving that there was no general depletion of antibodies during the procedure. The microarray images of the binding pattern of the sera before and after depletion experiments are shown in FIG. 7. Based on these data and the experiment using the monoclonal antibody, it was concluded that NMOP6 binds anti-AQP4 antibodies.

Peptoids NMOP2, NMOP5 and NMOP9 did not retain significant amounts of antibodies from the serum sample employed for the above depletion experiment. But this does not necessarily mean that they are ligands for antibodies that recognize antigens other than AQP4. The anti-AQP4 antibody spectrum in any patient is polyclonal. Given that a small molecule like a peptoid is likely to recognize only a fraction of this polyclonal population, it is possible that NMOP2, NMOP5 and NMOP9 are ligands for anti-AQP4 antibodies whose epitope selectivity is different than the antibodies recognized by NMOP6. Therefore, the depletion experiment was repeated with another serum sample obtained from an NMO patient that tested negative for anti-AQP4 antibodies by the cell-killing assay, but which showed readily detectable signals on these peptoids. This is the same serum sample used in the experiment show in FIGS. 2A-2C (second column).

As shown in FIG. 3, the signals on NMOP2, NMOP5 and NMOP9 were almost completely abolished by passage of the serum over immobilized AQP4 protein, similar to NMOP6. However, approximately 50% of the signal on these three peptoids was lost when the serum was passed over immobilized BSA. A reasonable interpretation of these results is that NMOP2, NMOP5 and NMOP9 are indeed ligands for anti-AQP4 antibodies that recognize different epitopes than the antibodies bound by NMOP6, but that these antibodies are “stickier” or more promiscuous in their binding selectivity. However, this issue will require more experimentation to address unequivocally.

Utility of the Peptoids as Diagnostic Reagents:

NMO patients are sometimes misdiagnosed as MS patients, with potentially serious adverse consequences (Uzawa et al., Eur. J. Neurol., 17:672-676, 2010). Since MS patients lack antibodies to AQP4, it was hypothesized that the peptoids isolated from the screen would not cross-react with antibodies from MS patients. As shown in FIG. 3, when serum from three MS patients was hybridized to the array, little or no signal was observed on any of the peptoids, though the DNP signal was strong, showing that the peptoids may be useful in distinguishing these sometimes symptomatically similar diseases from one another.

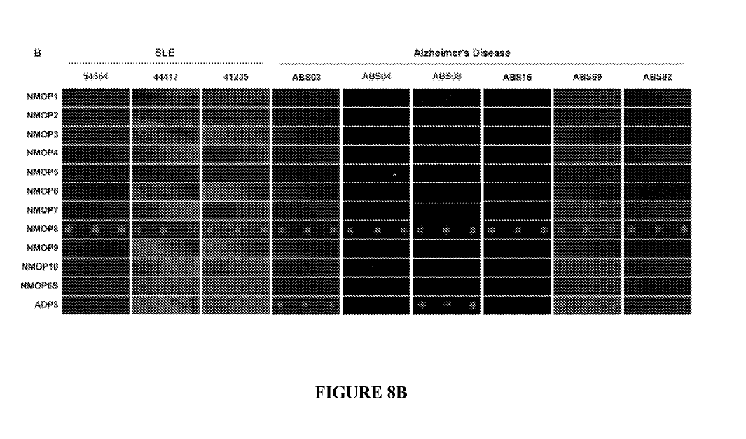

Since MS patients are not known to have high levels of autoantibodies, the peptoid probes were challenged with sera from diseases where autoantibodies have been detected or are thought to exist. For example, lupus patients are known to have unusually high levels of autoantibodies. Sera were also examined from patients with narcolepsy, which is believed to be an autoimmune condition (Hallmayer et al., 2009, Nature Genetics, 41:708-711), and Alzheimer's Disease (AD), where disease-related antibodies have been reported (Nagele et al., 2011, PLoS One 6, e23112; Reddy et al., 2011, Cell, 144:132-142). A total of 12 serum samples from these patients and six additional samples from normal control individuals were used. Each individual sample was applied to the array under the same conditions. None of the peptoids, with the exception of the NMOP8 control, showed significant signal with any of these samples above background, though it should be noted that the background was quite high with two of the lupus samples (FIGS. 8A and 8B). Peptoid ADP3, which was used as a control since it was previously reported to bind antibodies associated with AD, showed clear binding for some of the AD samples (FIGS. 8A and 8B). These data indicate that the peptoids identified in this study are indeed selective ligands for NMO antibodies.

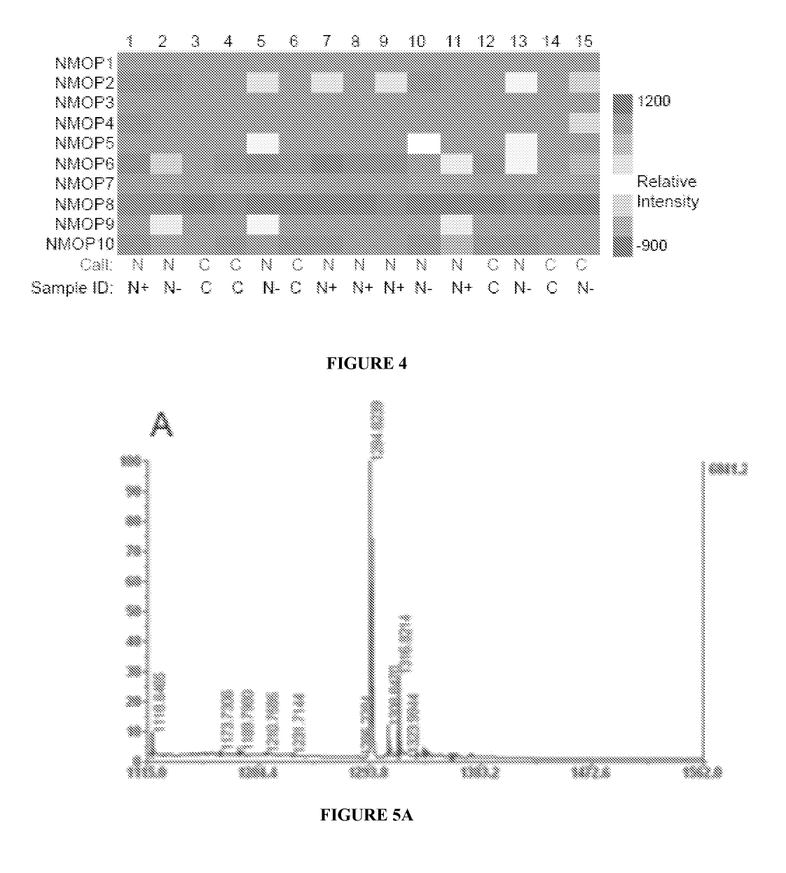

To determine the ability of the peptoids to accurately diagnose NMO more generally, an internally double-blinded study of 15 serum samples was conducted. The samples were applied to the array and called as NMO (N in FIG. 4) or controls (C in FIG. 4) based on the intensities observed at each feature. A positive call entailed observing a signal on any of the peptoids, other than NMOP8, that was above zero relative intensity or had a mean intensity greater than 50% of the intensity of NMOP8. The relative intensity was calculated by subtracting the mean intensity of each compound by the overall median intensity. The calls were then reported to the second individual who then obtained the key from the first technician to check the accuracy of the calls.

The data are shown in FIG. 4 in the form of a heat map. Beneath the map are the calls and the identities of the samples revealed after unblinding, including if the NMO serum samples tested positive or negative by the cell-killing assay (N⁺ or N⁻). The results showed that the panel of peptoids provides an accurate diagnostic test for NMO. 14 of the 15 samples were called correctly. Sample 15 was the exception. It was obtained from an NMO patient whose serum tested negative in the cell-killing assay. While peptoids NMOP2, NMOP4 and NMOP6 showed slight signals, these were very weak and did not pass the criteria for calling the sample NMO. In the other 14 cases, there was a very clear distinction between cases and controls, including all four of the other samples that were obtained from NMO patients whose antibodies did not mediate killing of AQP4-expressing cells. Indeed, NMOP6 alone correctly predicted 13 of the 15 samples (two false negatives).

Discussion

The previously reported novel technology for the discovery of serum biomarkers that involved comparative screens of the total complement of circulating IgG antibodies from case and control samples against a collection of synthetic, unnatural compounds (peptoids) (Reddy et al., 2011, Cell 144, 132-142). The goal was to identify compounds that retain antibodies that are present in much higher amounts in the serum of patients or animals with a particular disease and thus would serve as useful serum biomarkers for diagnosis. This approach was validated in EAE (experimental autoimmune encephalomyelitis), an animal model for MS induced by immunization with a self-antigen peptide derived from myelin oligodendrocyte glycoprotein (MOG). As hypothesized, peptoids that bound anti-MOG peptide antibodies were identified.

However, validation of this approach in a simple animal model does not equate with efficacy in the far more complicated arena of human disease. To address this issue, a study on sera from patients with Alzheimer's disease (AD) was performed. In a preliminary study of 54 patients, the same screening approach identified peptoids that captured antibodies found only in AD patients, not age-matched healthy controls or patients with Parkinson's disease. However, because these putative AD biomarker antibodies are novel the results cannot be taken as clear evidence of successful biomarker discovery until more extensive validation trials are completed. These are in progress.

NMO provides an excellent opportunity to identify disease-specific peptoid biomarkers in the context of an autoimmune disorder with a known antigenic target, AQP4. There is a strong expectation that application of this screening technology to NMO should identify peptoids that bind to anti-AQP4 antibodies. This was indeed the case. Peptoid NMOP6, one of several hits, was clearly identified as a ligand for anti-AQP4 antibodies based on its ability to bind a monoclonal anti-AQP4 antibody (FIG. 2B) and the ablation of signal from a serum sample that had been depleted of anti-AQP4 antibodies by passage over immobilized AQP4 (FIG. 3).

Note that peptoid NMOP6 cannot possibly mimic a native AQP4 epitope. In a peptoid, the side chains protrude from the sp²-hybridized nitrogen, rather than the sp³-hybridized α-carbon, of the main chain of the backbone. Moreover, most of the side chains in NMOP6 do not correspond to those found in proteins. Nonetheless, this relatively small molecule must recognize some conserved feature of the antigen-binding sites in some fraction of the anti-AQP4 polyclonal spectrum, though in the absence of structural data it is impossible to speculate on the detailed mode of binding.

From a preliminary blinded study (FIG. 4), it appears that peptoid NMOP6 and some of the other peptoid hits will be useful as diagnostic reagents. NMOP6 alone was able to call 13 of the 15 samples correctly. The two misses were NMO samples that tested negative in the cell-killing assay and thus might have low levels of anti-AQP4 antibodies, though other explanations cannot be ruled out, as stated above. These were called as false negatives. Sample 15, as discussed above, showed a very low signal and sample 10 essentially showed only background signal. However, a moderate signal was observed on NMOP5 for sample 10, allowing it to be called correctly. This was the only peptoid in the group that captured significant amounts of antibody from sample 10. Indeed, only peptoids NMOP5 and NMOP6 would have been necessary to achieve the 14/15 accuracy obtained in this initial study. NMOP2 and NMOP9 also displayed significant signals for several of the samples. Since these peptoids apparently bind different antibodies than NMOP6, it is likely that in larger studies the use of a four-peptoid panel might be advantageous. None of the peptoids exhibited significant signals when presented to 18 other samples, consisting of healthy controls and patients with lupus, MS, narcolepsy and Alzheimer's disease (FIGS. 8A and 8B). Lupus patients, in particular, are known to have high levels of several circulating autoantibodies, so this result supports the contention that the peptoids identified in this study are indeed selective ligands for NMO-related antibodies. Considering all of the samples analyzed in this study, the diagnostic sensitivity of the assay is 93% and the diagnostic specificity 100%. Note that for all of the samples, the signal obtained on the scrambled NMOP6s peptoid was negligible and the signal on DNP was high, providing useful negative and positive controls, respectively.

NMOP2, NMOP5 and NMOP9 may also bind to anti-AQP4 antibodies based on the serum depletion experiment (FIG. 3), but, if so, apparently to a different part of the polyclonal spectrum of this antibody population. A detailed characterization of the antibody-binding properties of these peptoids will require additional study.

Finally, it is noteworthy that this study employed a second generation screening technology different than the approach taken previously, which employed peptoids displayed on microarrays as the primary screening platform (Reddy et al., 2011, Cell 144, 132-142). Here bead-displayed peptoids made by solid-phase split and pool synthesis were employed (Figliozzi et al., 1996, Methods Enzymol 267, 437-447). Whereas the microarray technology limits the number of molecules that can be used in the primary screen to a few thousand, libraries of a few million compounds can be made on beads (Alluri et al., 2003, J Amer Chem Soc 125, 13995-14004). Studies of peptide libraries have demonstrated that larger libraries are more likely to contain high affinity ligands (Wilson et al., 2001, Proc Natl Acad Sci USA 98, 3750-3755), so this technological advance may be useful in the future for the discovery of superior antibody capture agents. This type of screening protocol has been applied to identify ligands for individual protein targets, but not for serum screening. A potential downside of this approach is that it involves the sequential screening of pooled serum samples (first control, then diseased), whereas the primary microarray screens involved exposing several microarrays to individual serum samples, both cases and controls, and identifying peptoids that “lit up” when exposed to the diseased samples, but not the controls. The danger of the pooling strategy is that one or more, but not all, of the diseased serum samples might contain high levels of a antibodies that are idiosyncratic to those individuals, but not be related to the disease of interest. In theory, if such antibodies were present at very high levels in even a single person in the pool one could identify peptoid ligands to it that would be mistaken as capture agents for disease-specific antibodies. Thus, the hits from the bead screen should be validated with several individual serum samples, which was done here using the array platform. This might explain why several of the peptoids identified as strong hits in the bead screen did not capture significant amounts of peptoids from any of the NMO serum samples.

### Example 2

### Competition ELISA Using NMO Serum

Experimental Conditions:

Incubate samples in for 1 hour at room temperature 1% BSA/PBST. Serum: 200 μg/ml total protein. For AC3054, 67 μg/ml total protein was used since the quantity of the sera was less.

Results:

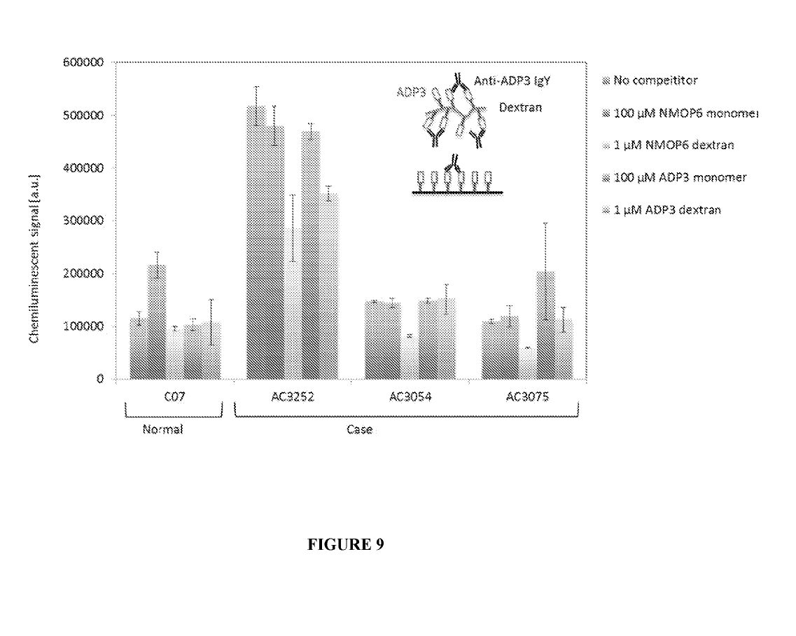

FIG. 9 shows that NMOP6-dextran competed with the signal of case sera but not normal sera at 1 μM. ADP3-dextran did not compete except for one case sera (AC3252). NMOP6 and ADP3 monomers did not compete at 100 μM.

ADP3 is a control peptoid that does not bind to antibodies in NMO patients. The difference between the signal measured when soluble dextran-NMOP6 is used as the competitor and when soluble dextran-ADP3 is used as the competitor is taken as the “real” signal.

The dextran polymer has, on average, about 20 peptoids coupled to it. As is apparent from the graph, even 1 μM of the polymer is a more efficient competitor than 100 μM of the monomeric peptoid. This is because the immobilized NMOP6 enjoys avidity effects in binding the bivalent antibodies, with which the soluble competitor must compete. Monomeric peptoids, of course, cannot bind with avidity, so a huge excess is necessary. The dextran-peptoid conjugate does show avidity effects, so binds to antibodies in solution at a much lower concentration. Other experiments have shown that the polymer binds somewhere between 500-1000-times better than the monomer to the NMO antibodies.

### Example 3

### Analysis of Peptoid NMOP6

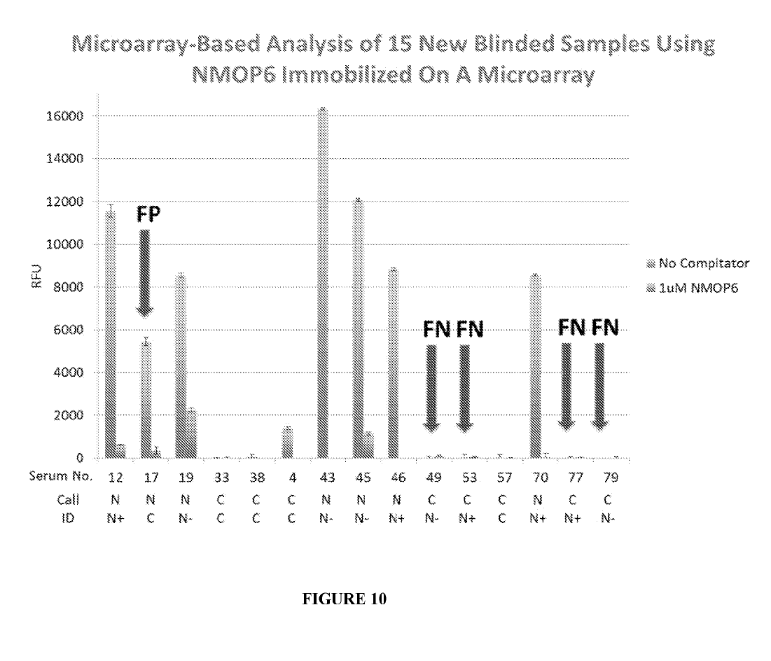

FIG. 10 shows the results from a microarray-based analysis of 15 new blinded samples using NMOP6 immobilized on a microarray. This blinded study of 15 serum samples was conducted using NMOP6 spotted onto microarrays. The signals were measured with and without blocking the serum with soluble NMOP6 prior to adding to the slide. False positives (FP) and false negatives (FN) are indicated. Note that the treatment status of most of these samples was unknown.

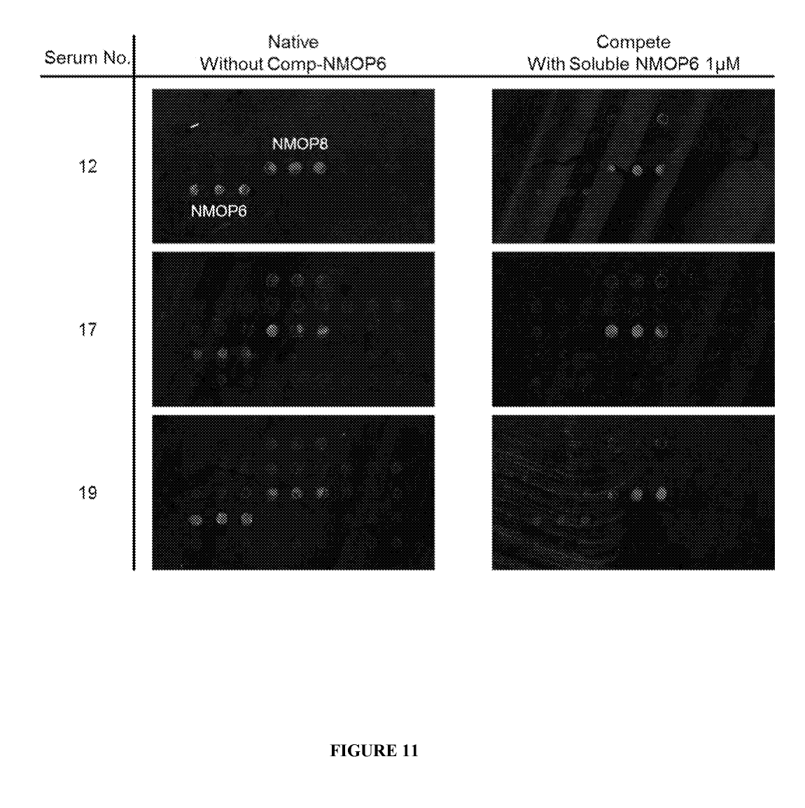

FIG. 11 shows a selected microarray image of an NMOP6 competition assay. NMOP8 is a peptoid that binds directly to the secondary antibody, so it is always bright and is not competed by soluble NMOP6.

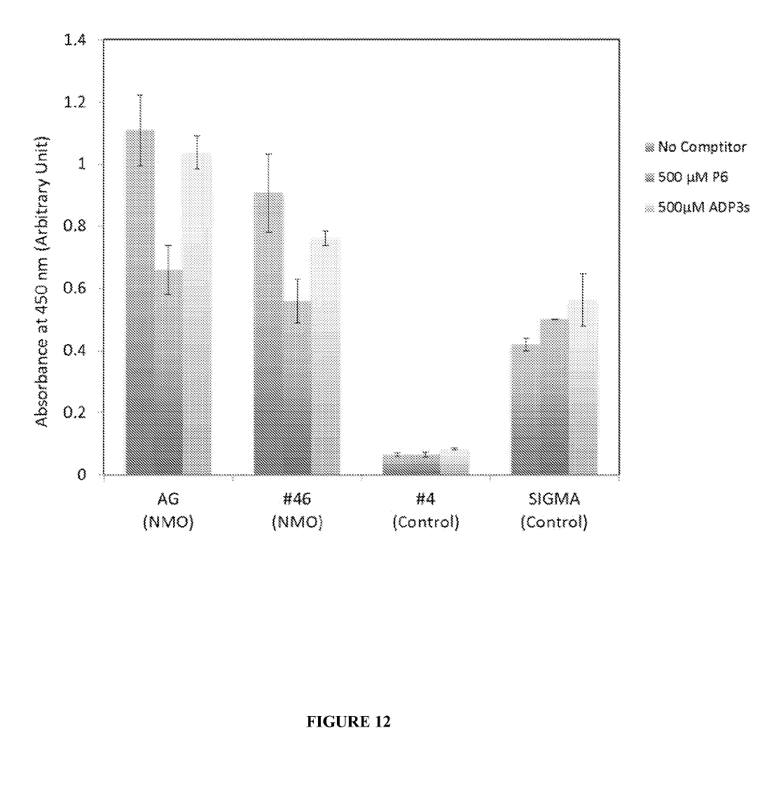

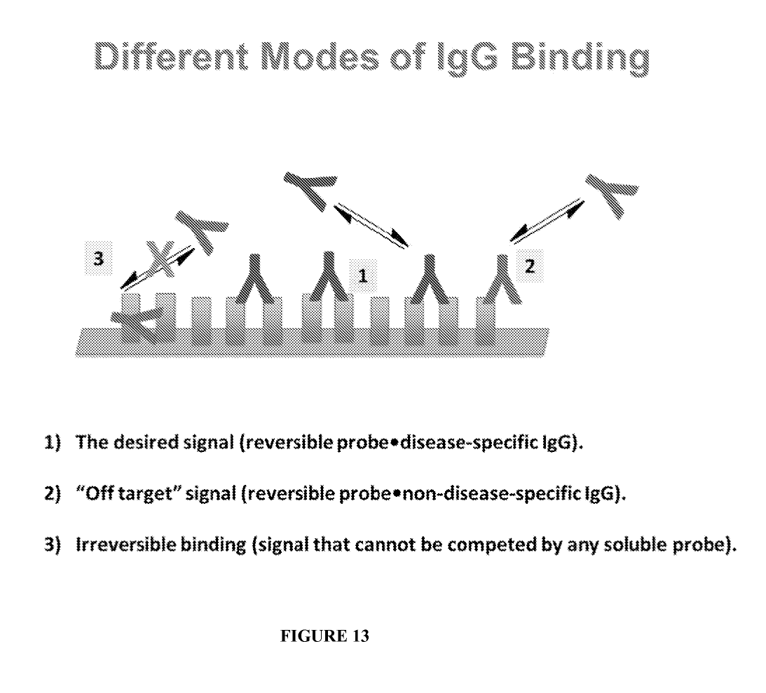

FIG. 12 shows ELISA data using immobilized NMOP6 and the indicated soluble competitor. The results obtained are typical results seen in ELISA assays. The blue bar is the uncorrected raw signal observed at a particular serum concentration. The red bar is the signal observed when the serum sample is pre-incubated with soluble NMOP6, which should bind up most of the antibodies that recognize this peptoid specifically. The green bar represents the signal when the serum sample is pre-incubated with a control peptoid (ADP3s) that should not bind selectively to the NMOP6 peptoid. As the data show, the total signal is made up of three components (see FIG. 13 for an illustration): 1) Specific, competable antibody binding to immobilized NMOP6, 2) Non-specific, competable binding of antibodies to immobilized NMOP6, 3) Non-specific and non-competable binding of IgG antibodies to immobilized NMOP6. The difference between the red and green bars is the true signal, so long as they are both smaller than the blue bar.

TABLE 3
Summary of ELISA data in the blinded study. 20/23 samples were
correct with 0 false positives and 3 false negatives.

| Sl | SAMPLES | Corrected | | |
|---|---|---|---|---|
| No | ACP2# | Signal | CALL | ID |
| 1 | 4 | 0.0183 | C | C |
| 2 | 6 | −0.007 | C | C |
| 3 | 11 | 0.006 | C | MS |
| 4 | 12 | 0.1915 | NMO | NMO+ |
| 5 | 17 | 0.001 | C | C |
| 6 | 19 | 0.128 | NMO | NMO− |
| 7 | 33 | −0.06 | C | C |
| 8 | 38 | 0.033 | C | C |
| 9 | 43 | 0.28 | NMO | NMO− |
| 10 | 45 | 0.079 | NMO | NMO− |
| 11 | 46 | 0.203 | NMO | NMO+ |
| 12 | 49 | 0.031 | C | NMO− |
| 13 | 53 | −0.018 | C | NMO+ |
| 14 | 57 | −0.011 | C | C |
| 15 | 70 | 0.234 | NMO | NMO+ |
| 16 | 77 | −0.039 | C | NMO+ |
| 17 | 79 | 0.034 | C | MS |
| 18 | 103 | 0.06 | C | C |
| 19 | AG | 0.377 | NMO | NMO |
| 20 | TG | 0.115 | NMO | NMO |
| 21 | SS | 0.1505 | NMO | NMO |
| 22 | SA | 0.1853 | NMO | NMO |
| 23 | JB | 0.181 | NMO | NMO |

ELISA data of the NMO samples on NMOP6 immobilized plate.

In order to avoid preparing large amounts of peptoids a different approach was taken. Based on previous experiments, it was found that immobilized antibodies enjoy an avidity effect, which greatly increases their affinity for the antibodies relative to soluble competitor peptoid. Thus, the effect of competing with soluble dextran polymers of the peptoid was examined, which also can bind antibodies cooperatively. As shown here, this greatly reduced the amount of soluble competitor needed.

Table 4 is a summary of data obtained.

| | NMOP6 | NMOP6 | | | |
|---|---|---|---|---|---|
| Serum ID | Signal | Comp | Ratio | Call | ID |
| 4 | 1373 | −44 | 31 | C | C |
| 12 | 11548 | 599 | 19 | NMO | NMO+ |
| 17 | 5432 | 340 | 15 | NMO | C |
| 19 | 8532 | 2230 | 3 | NMO | NMO− |
| 33 | −21 | −11 | 1 | C | C |
| 38 | 55 | −154 | 0 | C | C |
| 43 | 16328 | −32 | 510 | NMO | NMO− |
| 45 | 12062 | 1139 | 10 | NMO | NMO− |
| 46 | 8823 | −15 | 588 | NMO | NMO+ |
| 49 | −63 | 110 | 0 | C | NMO− |
| 53 | 9 | 51 | 0 | C | NMO+ |
| 57 | 9 | −4 | 2 | C | C |
| 70 | 8546 | 9 | 949 | NMO | NMO+ |
| 77 | 7 | 14 | 0 | C | NMO+ |
| 79 | −56 | −18 | 3 | C | NMO− |
| 1 | 1844 | N/A | N/A | NMO | NMO+ |
| 2 | 1259 | N/A | N/A | NMO | NMO+ |
| 3 | 88 | N/A | N/A | C | C |
| 4 | 55 | N/A | N/A | C | C |
| 5 | 1848 | N/A | N/A | NMO | NMO− |
| 6 | 68 | N/A | N/A | C | C |
| 7 | 1885 | N/A | N/A | NMO | NMO+ |
| 8 | 1863 | N/A | N/A | NMO | NMO+ |
| 9 | 1857 | N/A | N/A | NMO | NMO+ |
| 10 | 82 | N/A | N/A | NMO | NMO− |
| 11 | 672 | N/A | N/A | NMO | NMO− |
| 12 | 54 | N/A | N/A | C | C |
| 13 | 947 | N/A | N/A | NMO | NMO− |
| 14 | 78 | N/A | N/A | C | C |
| 15 | 272 | N/A | N/A | NMO | NMO− |
| 30 | 2197 | N/A | N/A | NMO | NMO+ |
| 35 | 2148 | N/A | N/A | NMO | NMO+ |
| 68 | 2761 | N/A | N/A | NMO | NMO+ |
| 84 | 1845 | N/A | N/A | NMO | NMO+ |
| 15 | 800 | N/A | N/A | NMO | NMO− |
| 31 | 792 | N/A | N/A | NMO | NMO− |
| 74 | 2557 | N/A | N/A | NMO | NMO− |
| 81 | 1115 | N/A | N/A | NMO | NMO− |

1. A method of identifying ligands which specifically bind to receptors associated with autoimmune diseases, the method comprising:
providing a compound library of candidate ligands wherein each ligand is coupled to a support;
contacting the library with a control sample and removing ligands associated with non-specific binding and/or specific ligands against antibodies common in healthy people;
contacting the remaining ligands with a test sample and a labeled secondary antibody; and, identifying ligands which specifically bind to receptors associated with autoimmune diseases.

2. The method of claim 1, wherein the ligands are peptoids of at least a 3-mer comprising a general structural formula (I): 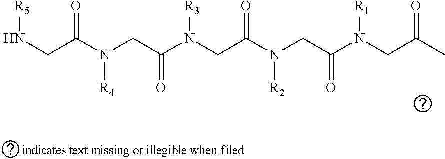
Wherein
R₁, R₂, R₃, R₄, R₅ independently comprise one or more groups derived from amines comprising:

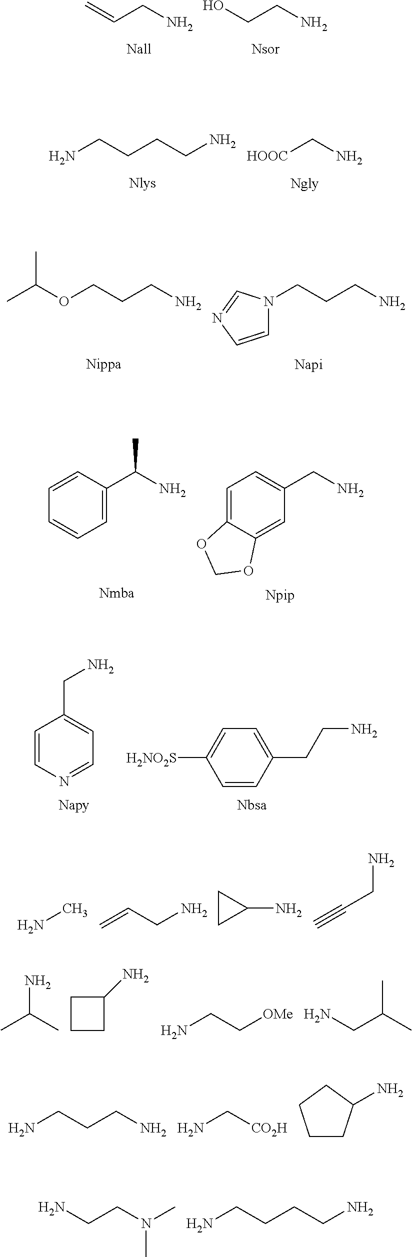

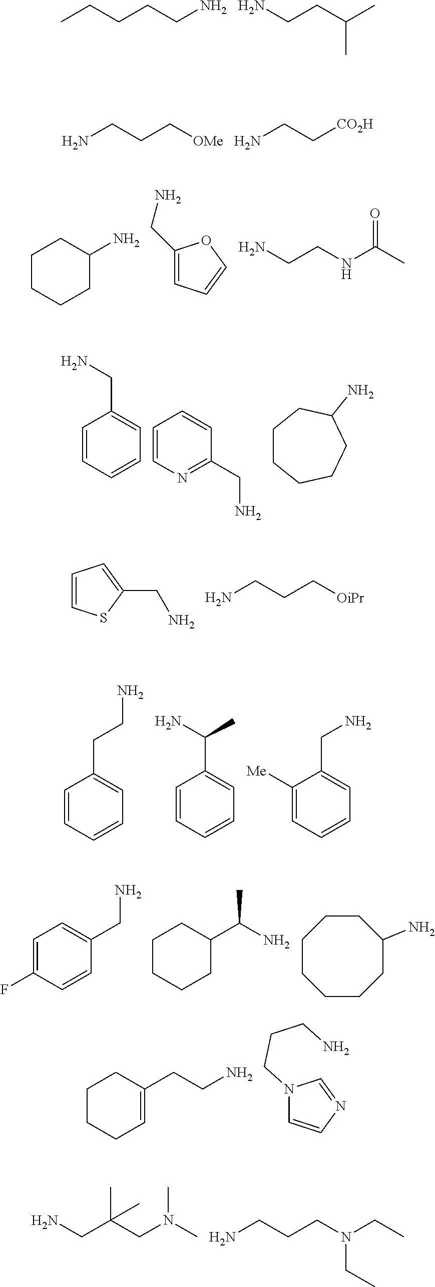

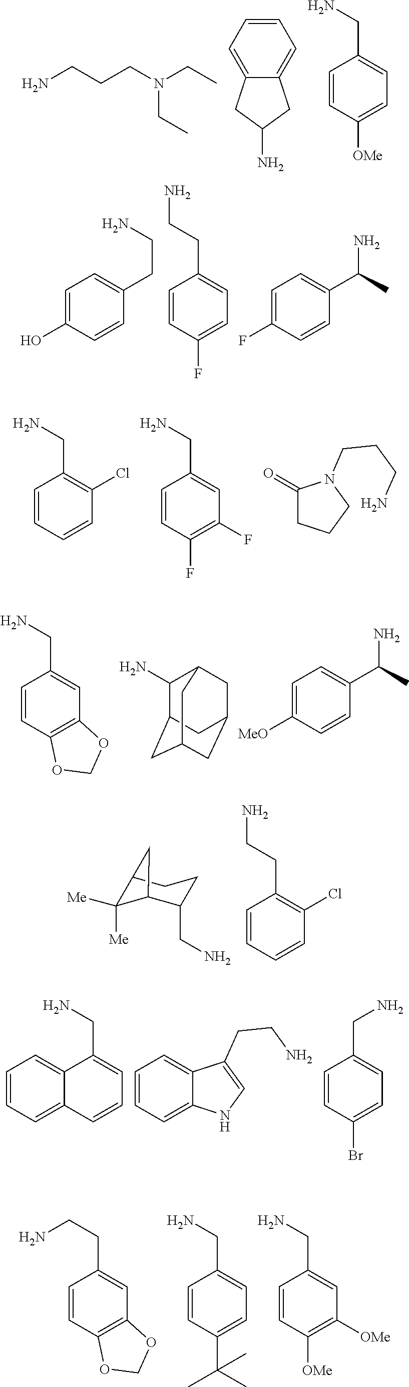

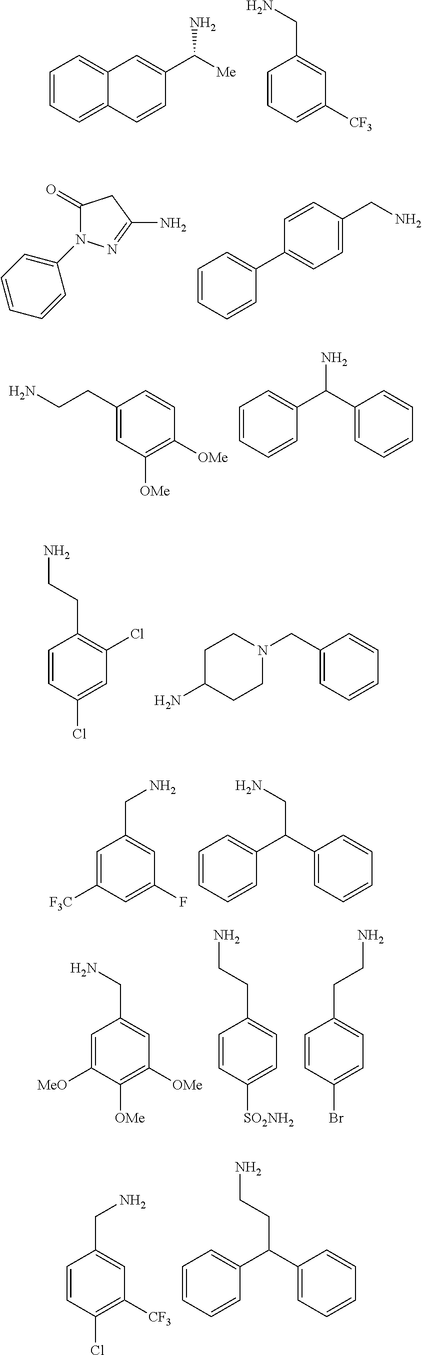

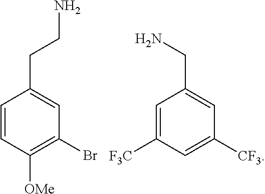

3. The method of claim 2, wherein the peptoid of formula (I) is attached to a bead via a linker molecule wherein the linker molecule is non-variable.

4. The method of claim 1, wherein the receptors comprise antibodies, T cell receptors or molecules associated with an autoimmune response.

5. The method of claim 4, wherein the receptor is an antibody.

6. The method of claim 5, wherein the antibody is specific for aquaporin 4 (AQP4).

7. The method of claim 1, wherein the substrate comprises: bead, a chip, a filter, a dipstick, a membrane, a polymer matrix or a well.

8. A peptoid comprising formula (I): 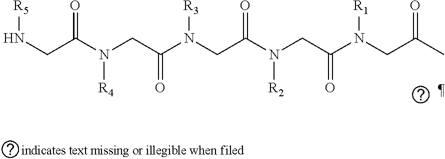
Wherein:
R₁, R₂, R₃, R₄, R₅ independently comprise one or more groups derived from amines comprising:

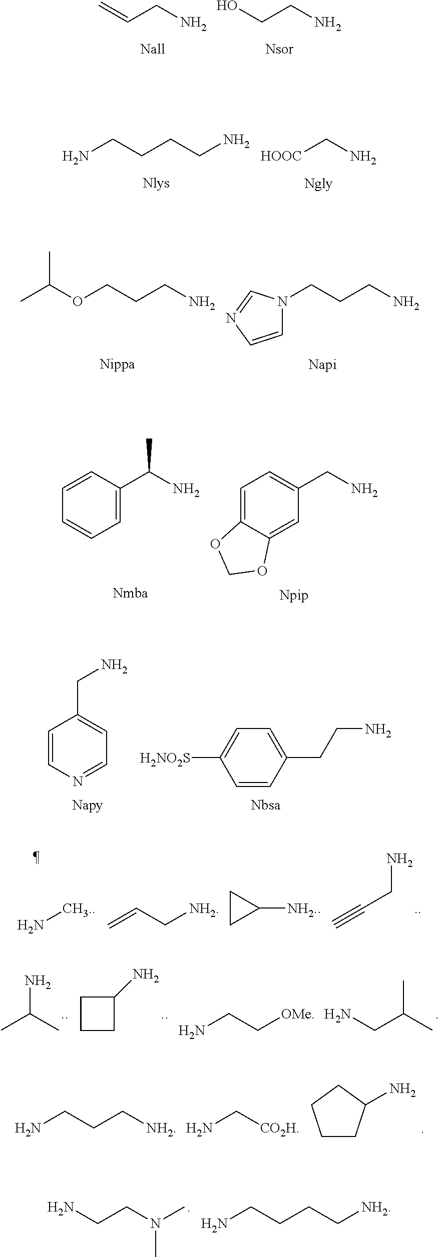

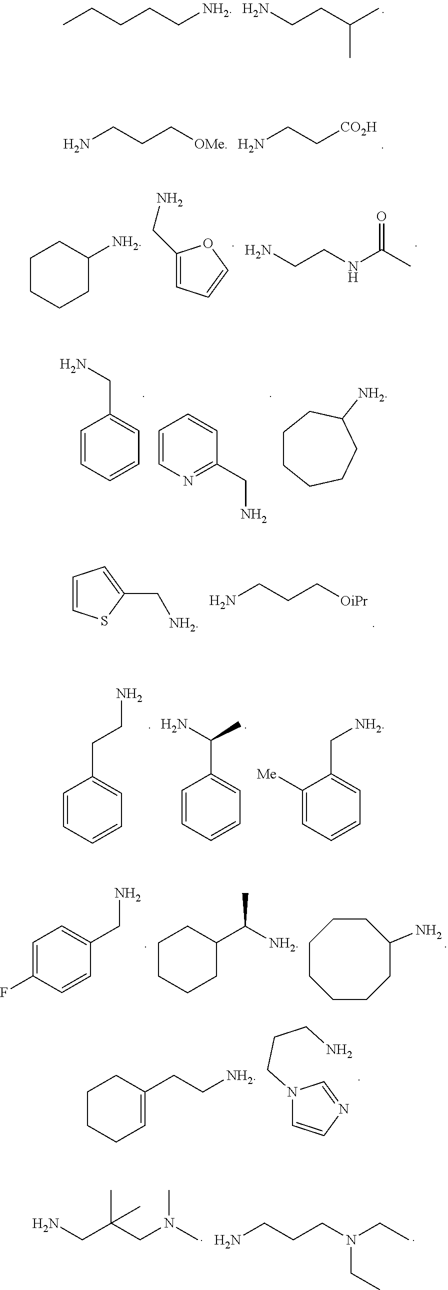

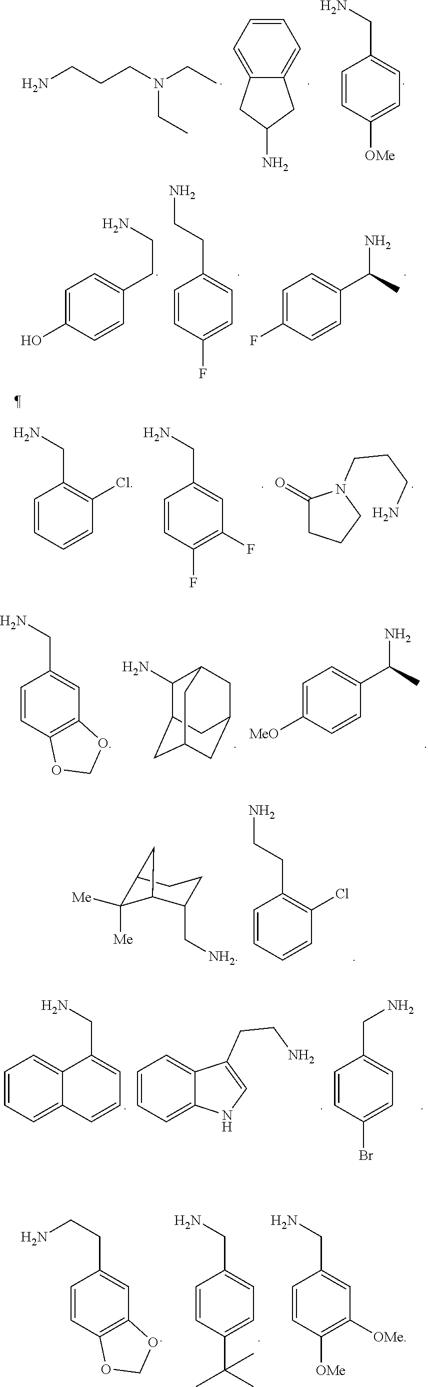

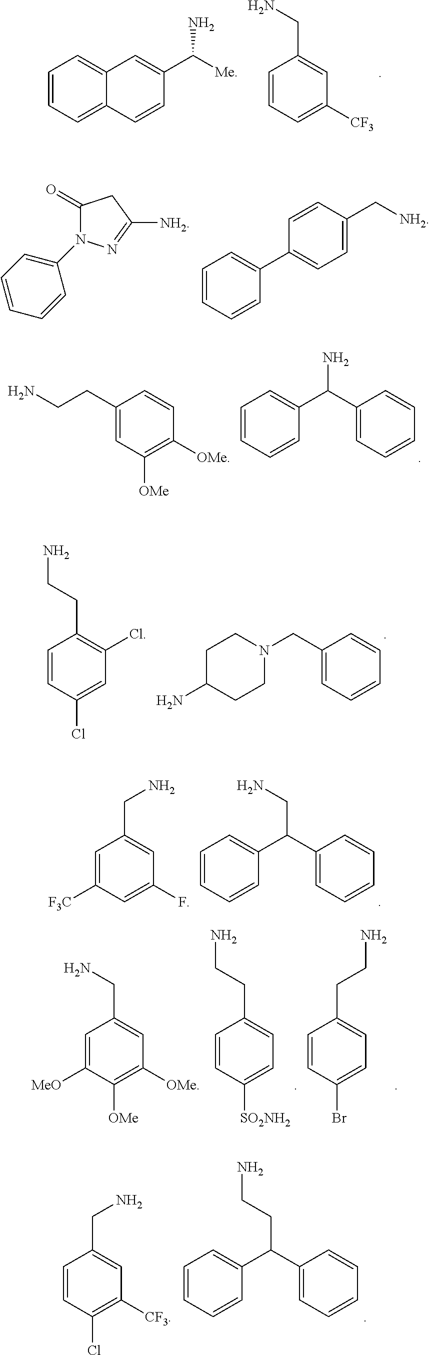

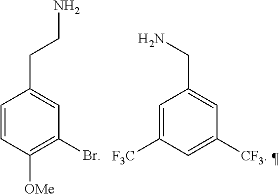

9. A combinatorial library of compounds comprising a plurality of peptoid molecules each having at least one unit of formula (I): 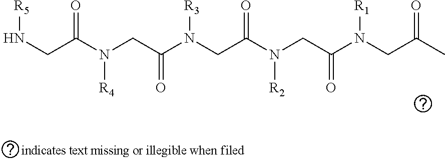
Wherein:
R₁, R₂, R₃, R₄, R₅ independently comprise one groups derived from amines comprising:

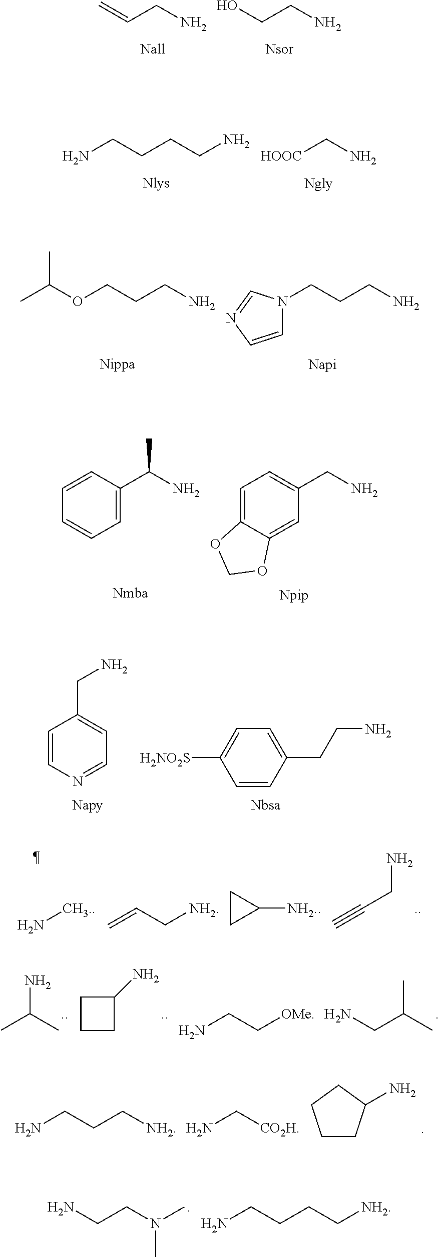

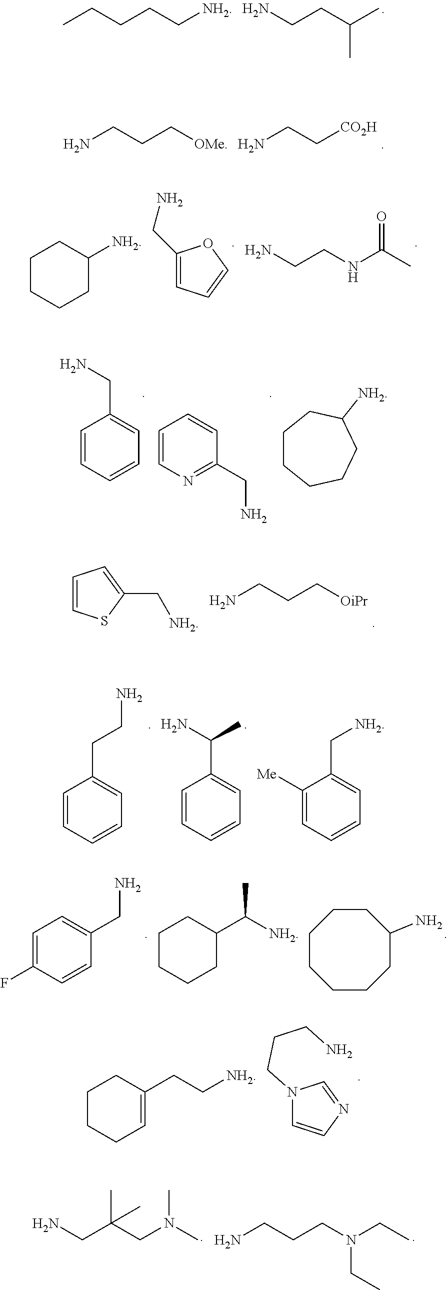

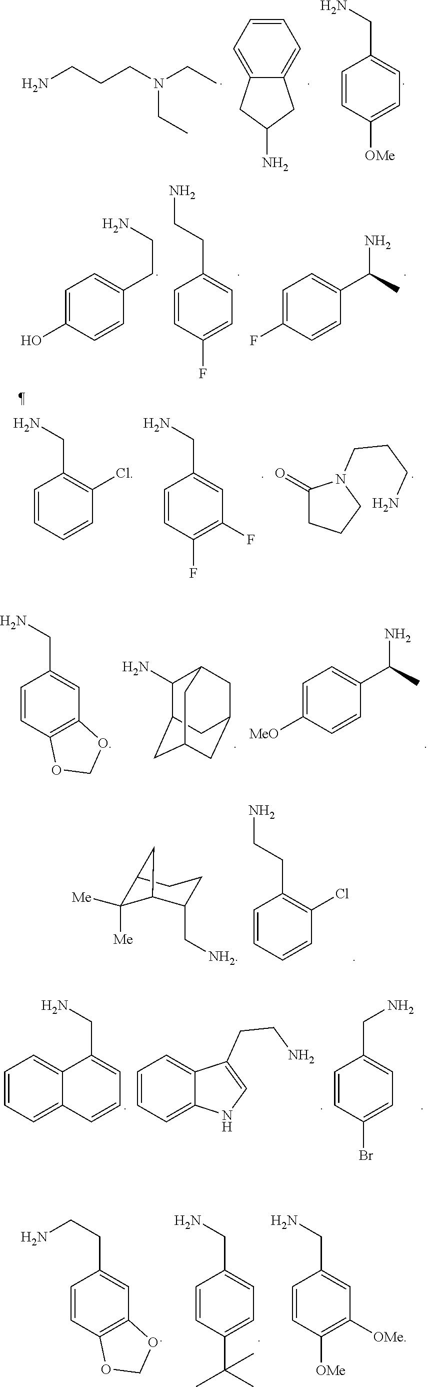

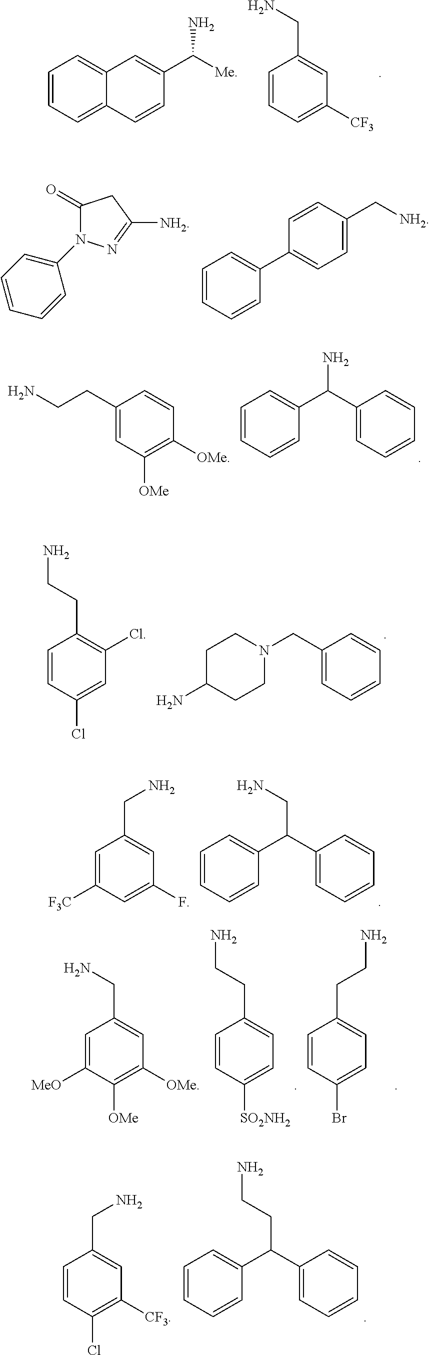

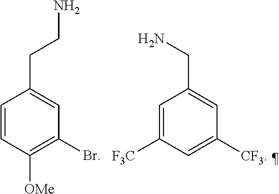

10. The combinatorial library of claim 8, wherein a peptoid of formula I comprises:

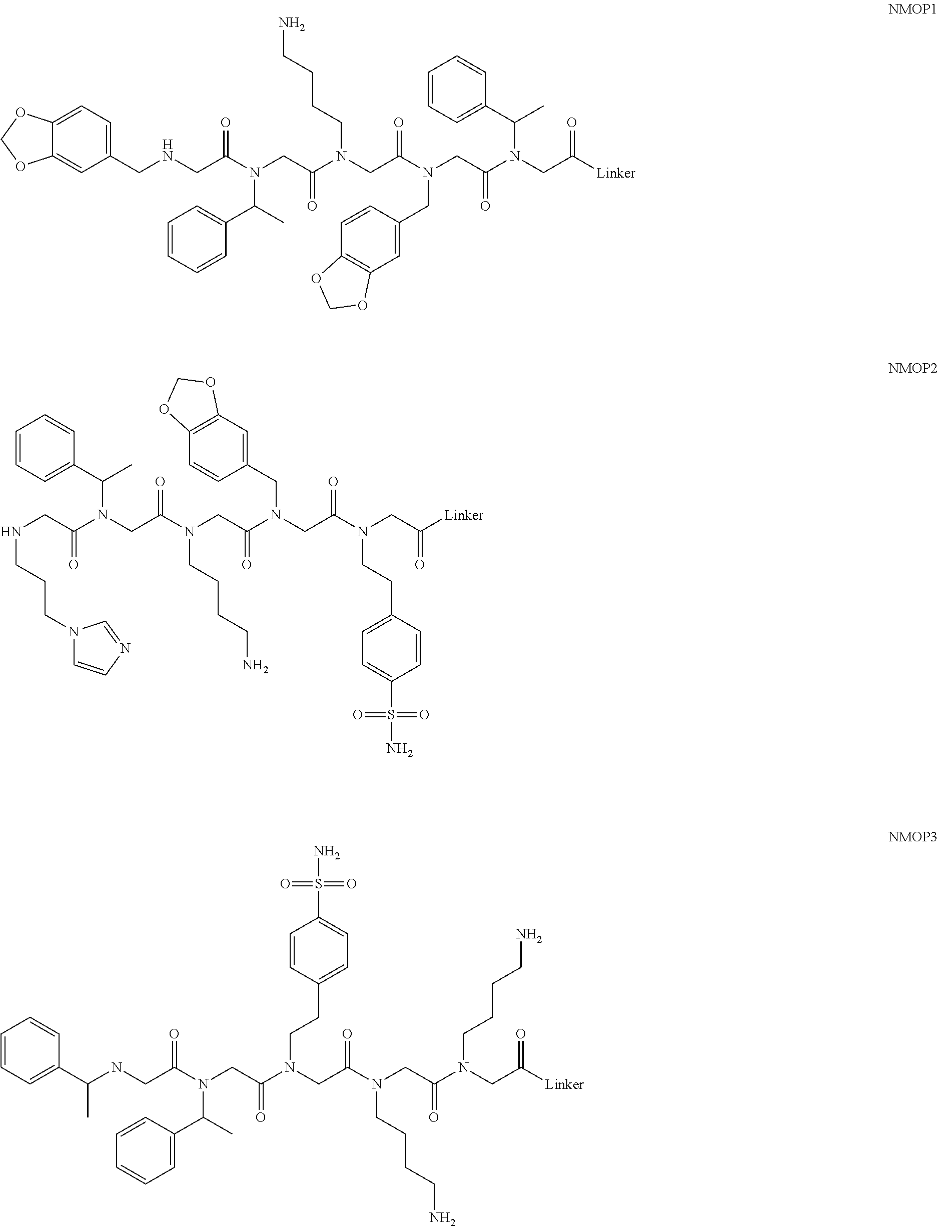

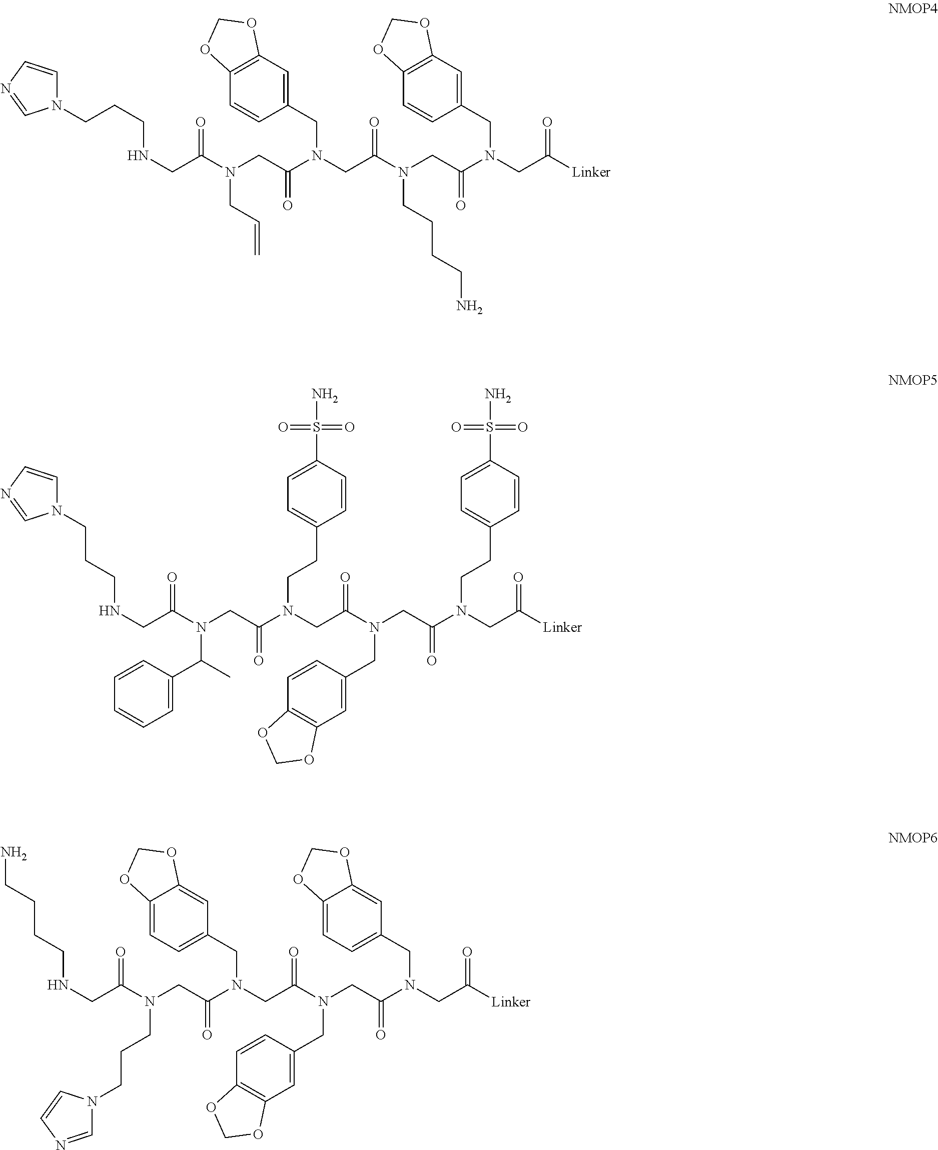

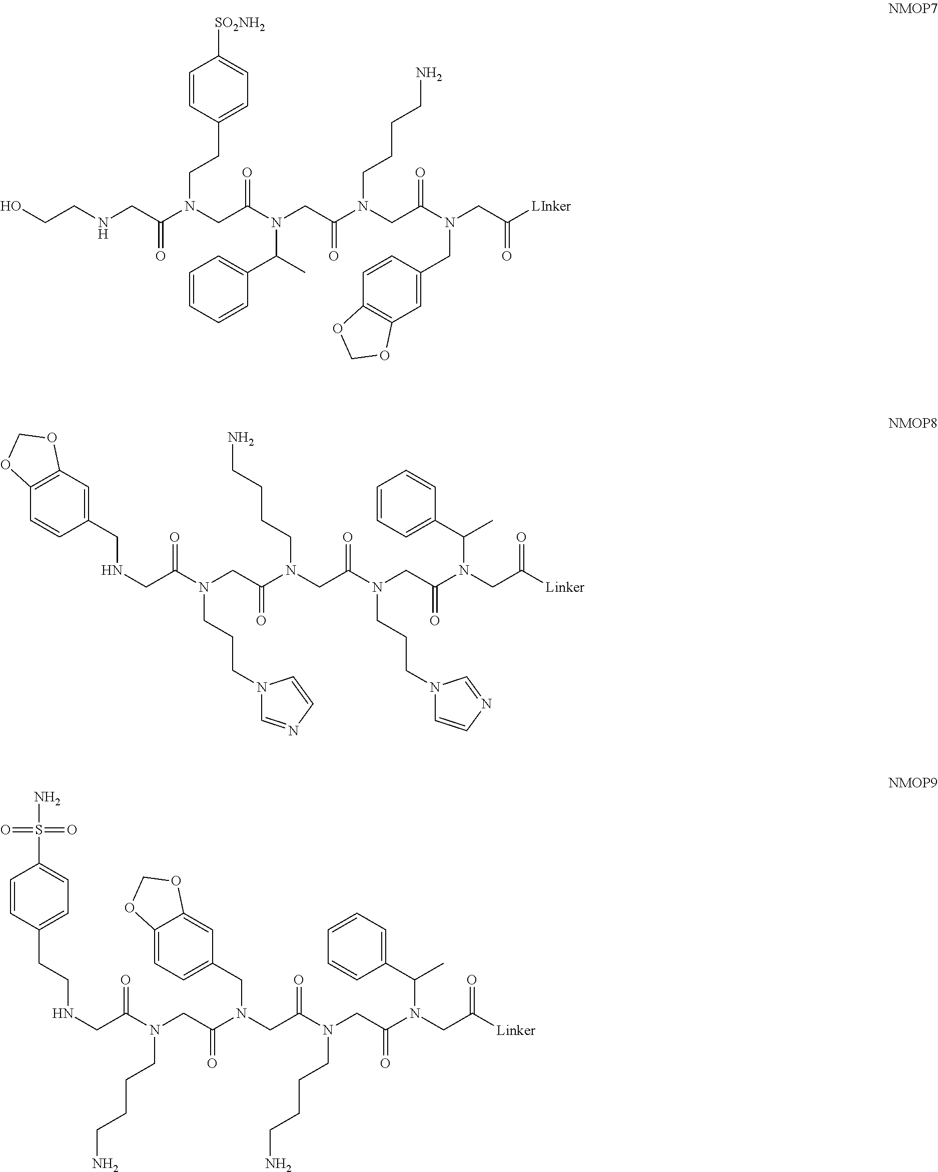

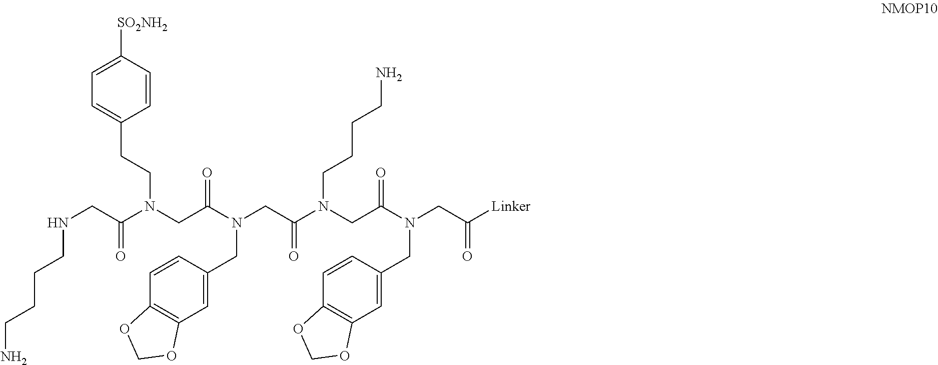

11. A peptoid combinatorial library produced by the method according to claim 1.

12. A method of diagnosing Neuromyelitis optica (NMO) comprising:
contacting a patient sample with a combinatorial library of peptoids, wherein peptoids which specifically bind to antibodies specific for aquaporin 4 (AQP4) or NMO antigens, are detected.

13. The method of claim 12, wherein an assay for diagnosing NMO comprises: immunoassays, ELISA assays, competitive ELISA assays, enzyme assays, bioassays, biochip assays, blots, hybridization assays, cell-based assays, high-throughput screening assays, chromatography, chemical assays, phage display assays, lab-on-a-chip, microfluidics based assays, microarrays, microchips, nanotube based assays, colorimetric assays, spectrophotometric assays or combinations thereof.

14. The method of claim 12, wherein the one or more peptoids comprise a detectable moiety, the detectable moiety comprising: a luminescent moiety, a chemiluminescent moiety, a fluorescence moiety, a bioluminescent moiety, an enzyme, a natural or synthetic moiety.

15. The method of claim 12, wherein the peptoids comprise:

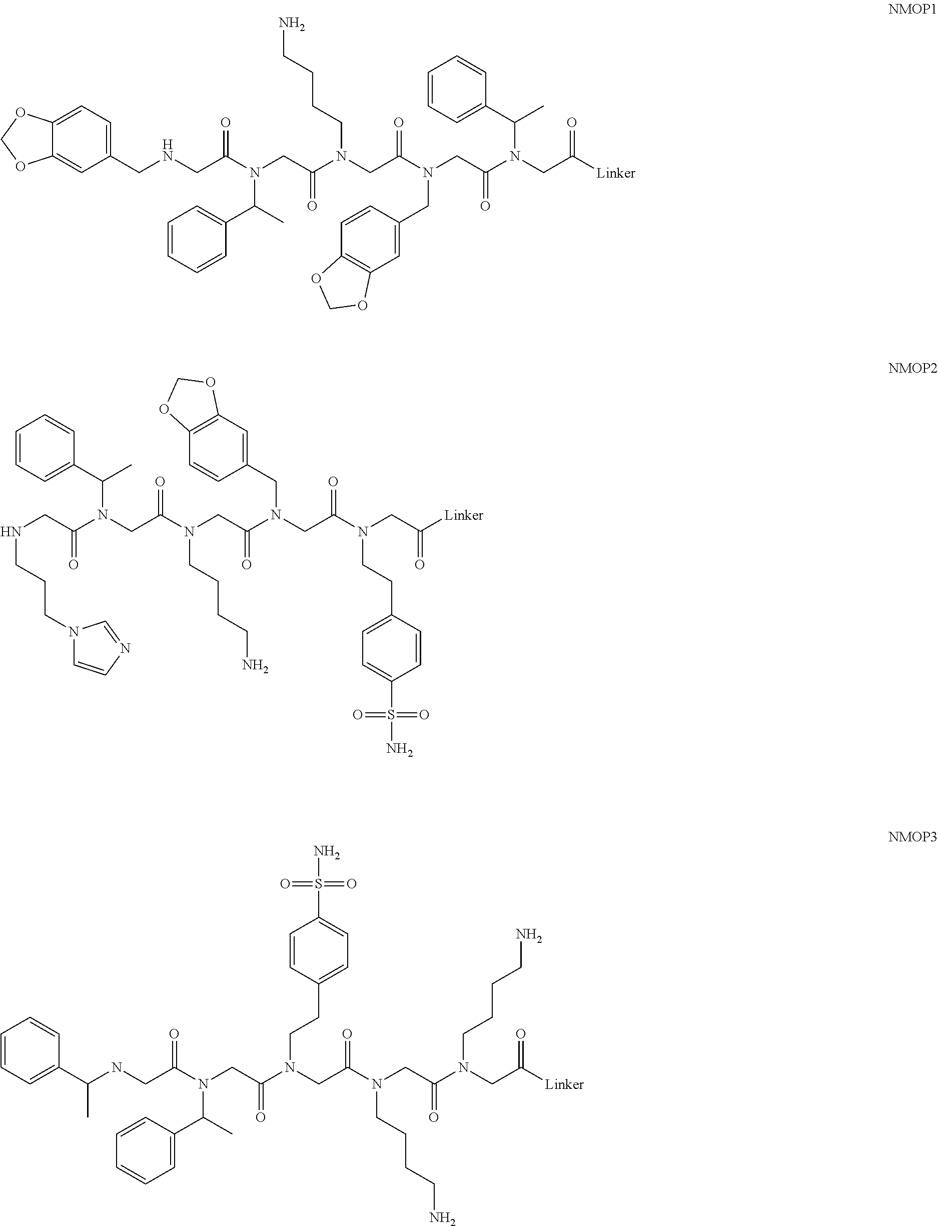

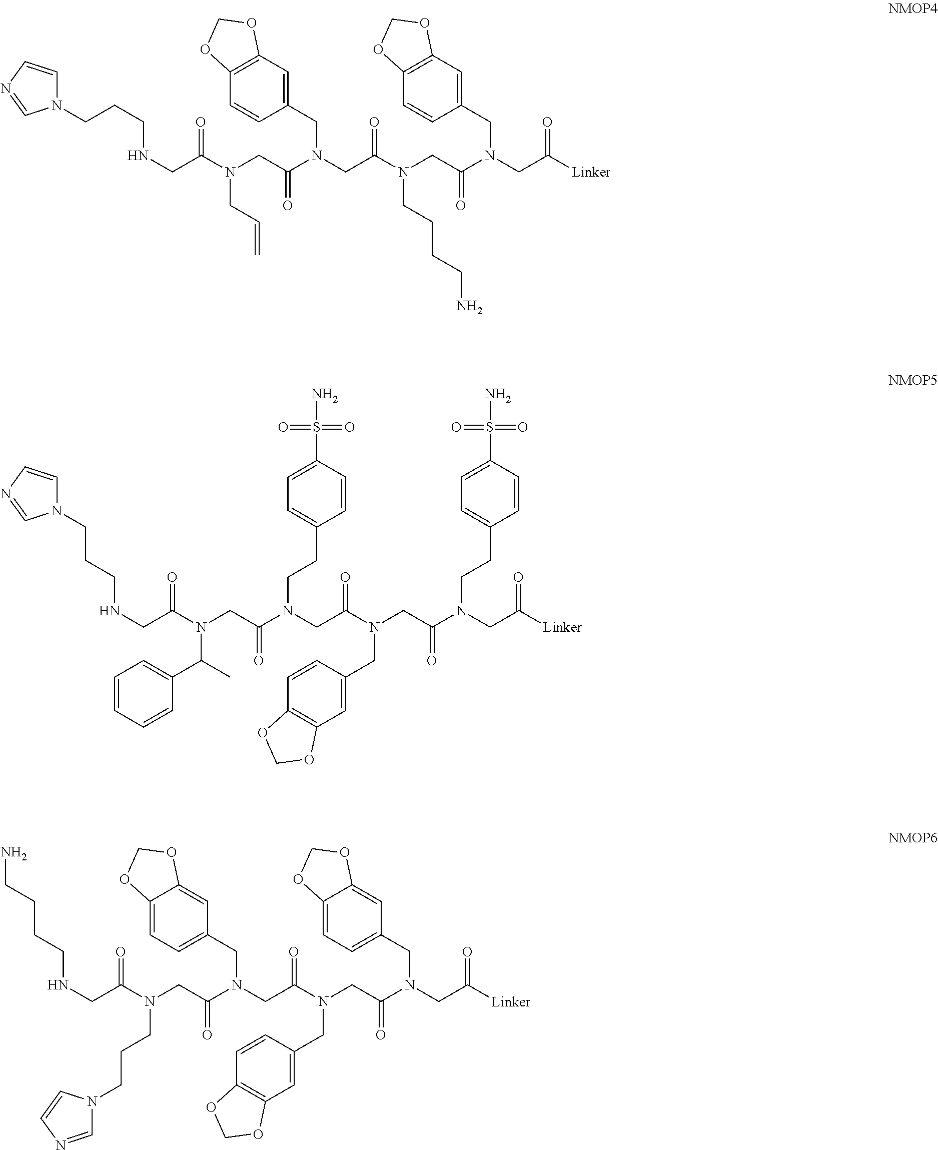

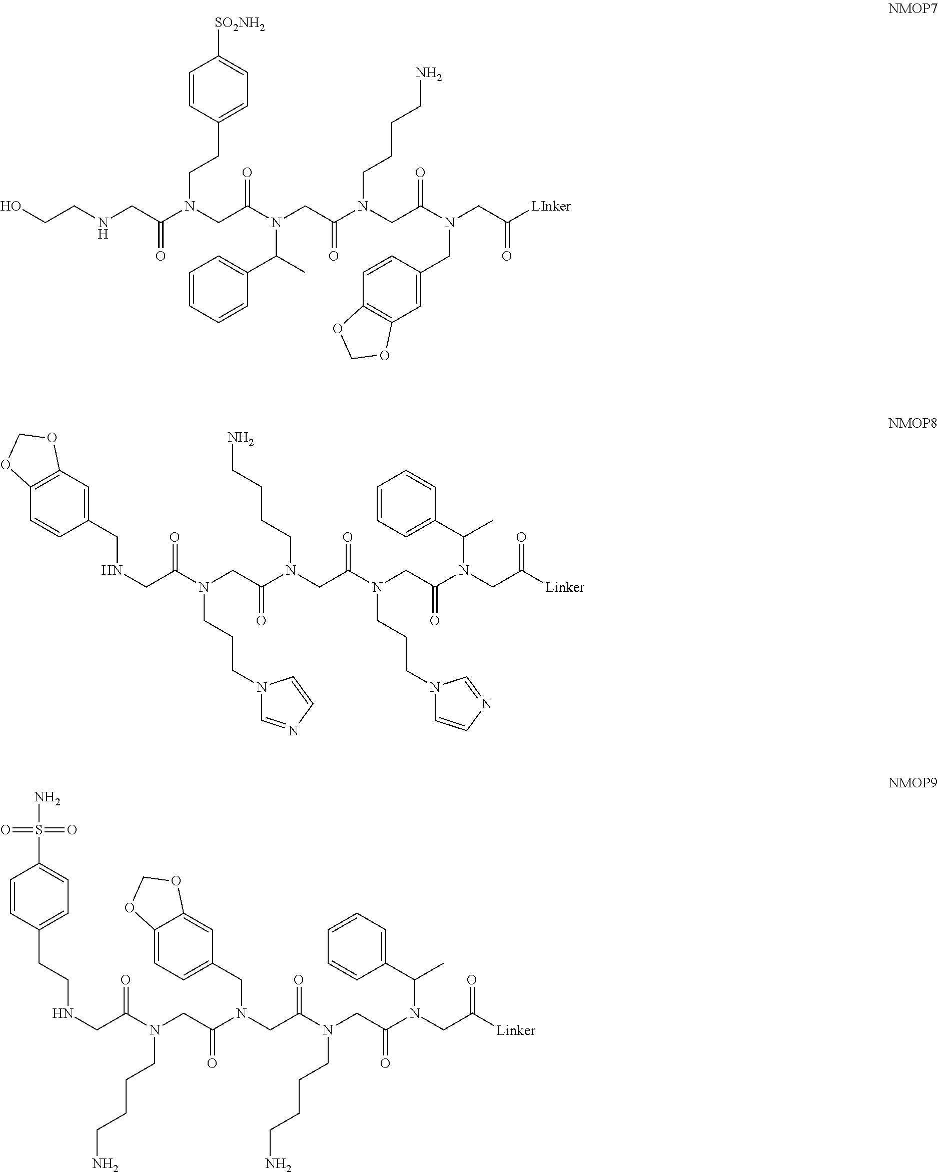

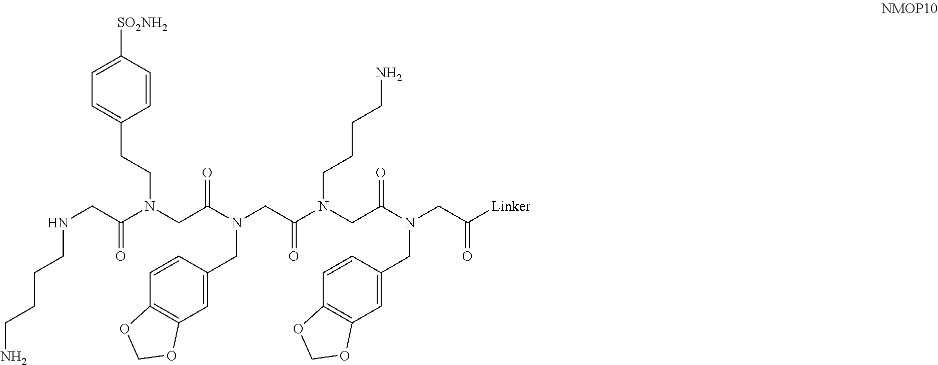

16. A polymer comprising one or more monomers, the one or more monomers comprising one or more ligands, wherein the ligands are peptoids of at least 5-mer having a general structural formula (I): 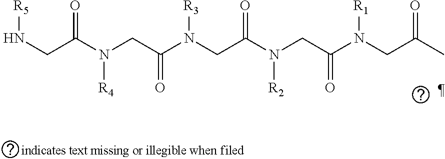
wherein
R₁, R₂, R₃, R₄, R₅ independently comprise one or more groups derived from amines comprising:

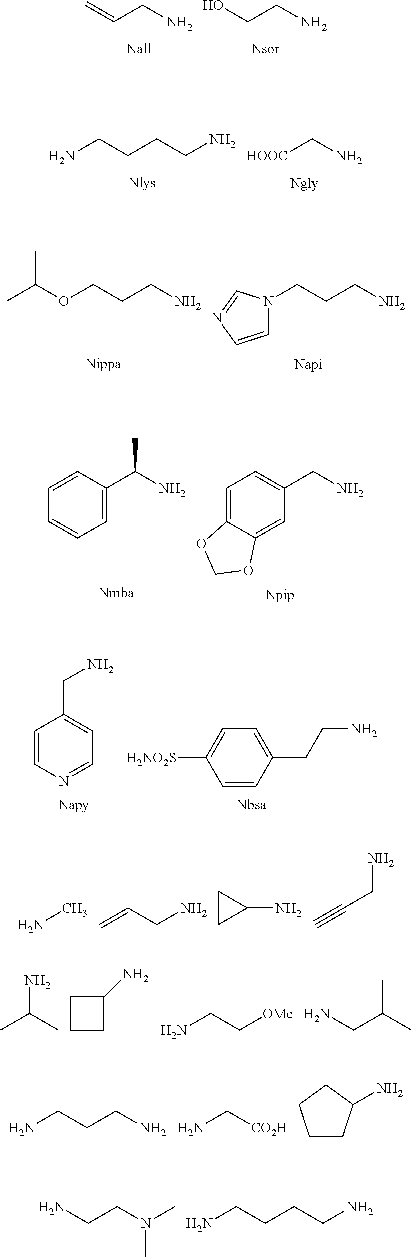

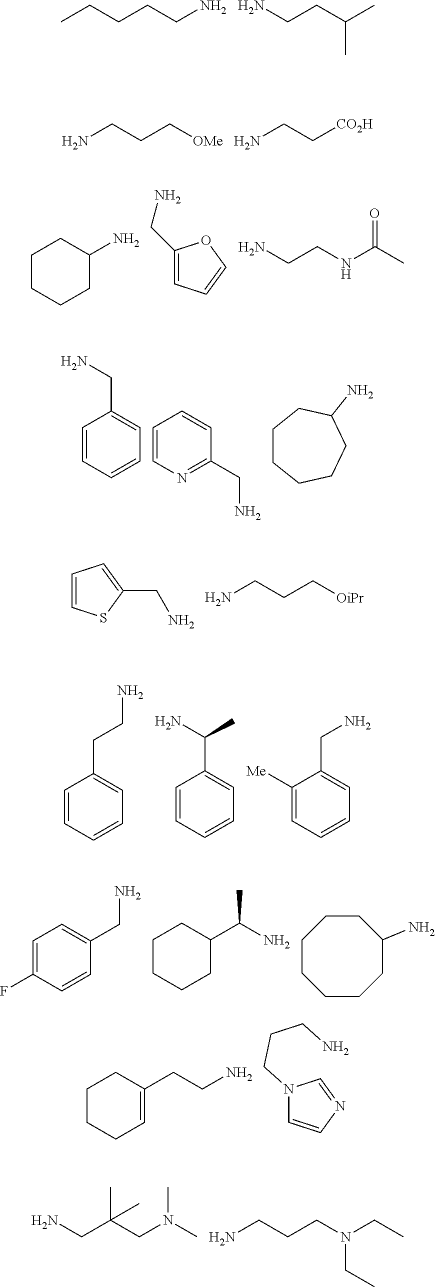

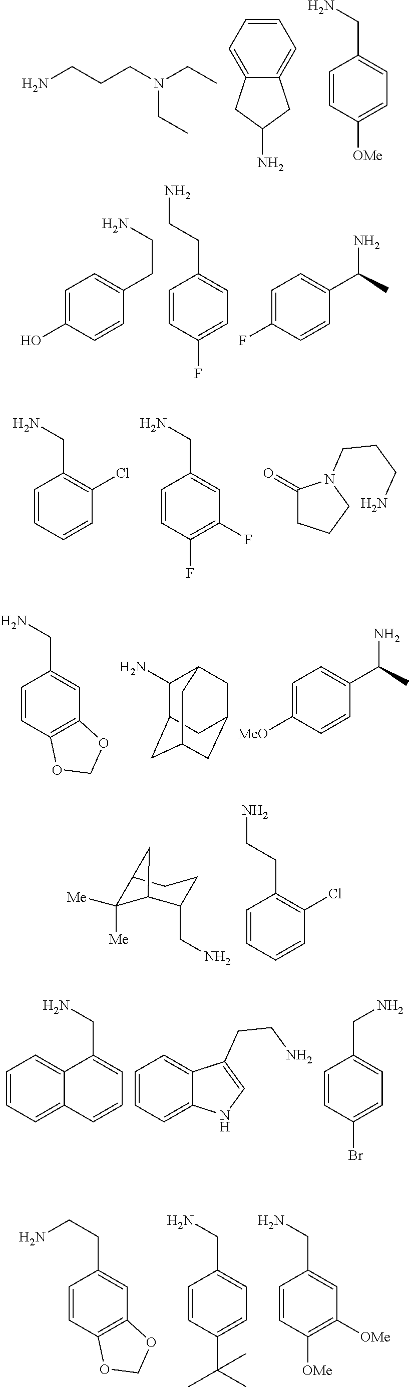

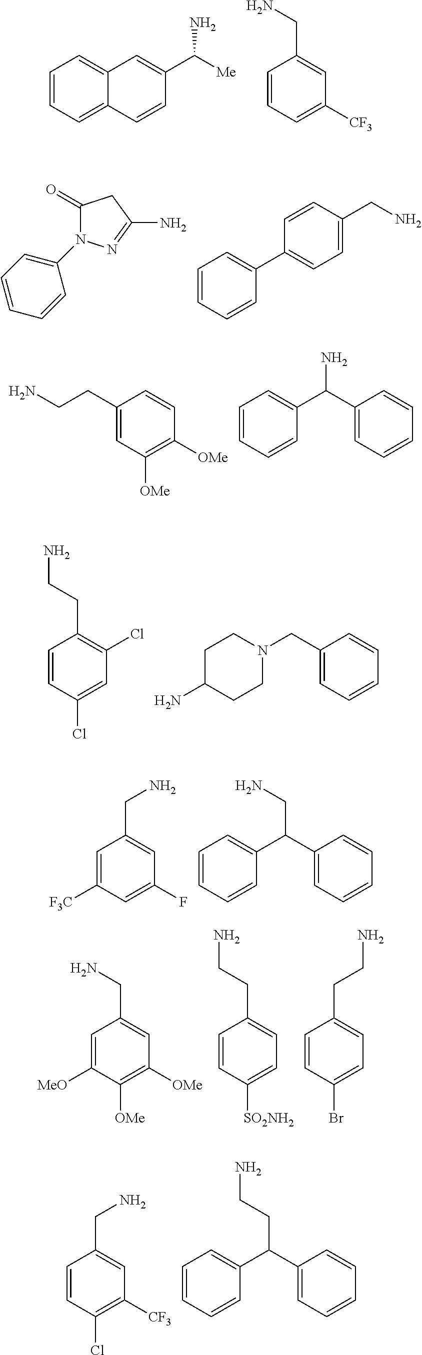

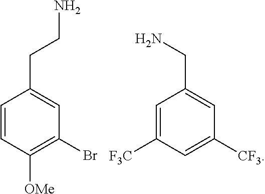

17. The polymer of claim 16, wherein the monomers comprise: dextran, amino acids, peptide nucleic acids, nucleic acids, synthetic molecules, organic or inorganic molecules, carbohydrates, variants or combinations thereof.

18. The polymer of claim 17, wherein the dextran is linear, branched, or combinations thereof.

19. The polymer of claim 18, wherein the dextran further comprises one or more modified dextran molecules.

20. The polymer of claim 16, wherein the polymer comprises at least two peptoids of general structural formula I.

21. The polymer of claim 16, wherein the polymer is a homopolymer, heteropolymer or copolymer.

22. A method of diagnosing a disease or disorder comprising:
obtaining a biological sample;
incubating the biological sample with a ligand;
detecting ligands specifically bound to a specific disease antigen, thereby diagnosing the disease or disorder.

23. The method of claim 22, wherein the ligands are peptoids of at least 5-mer comprising a general structural formula (I): 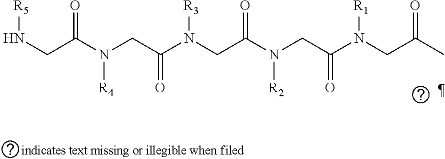
Wherein
R₁, R₂, R₃, R₄, R₅ independently comprise one or more groups derived from amines comprising:

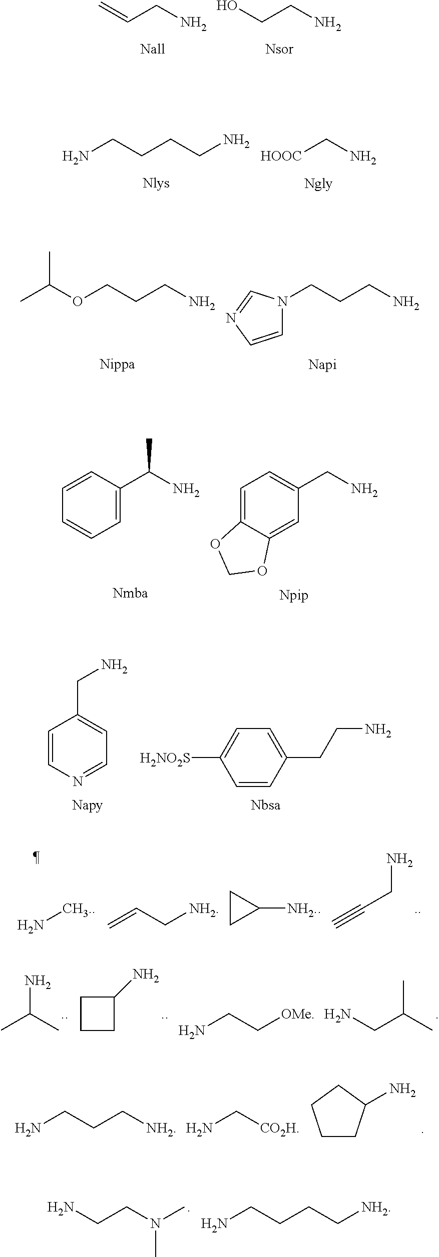

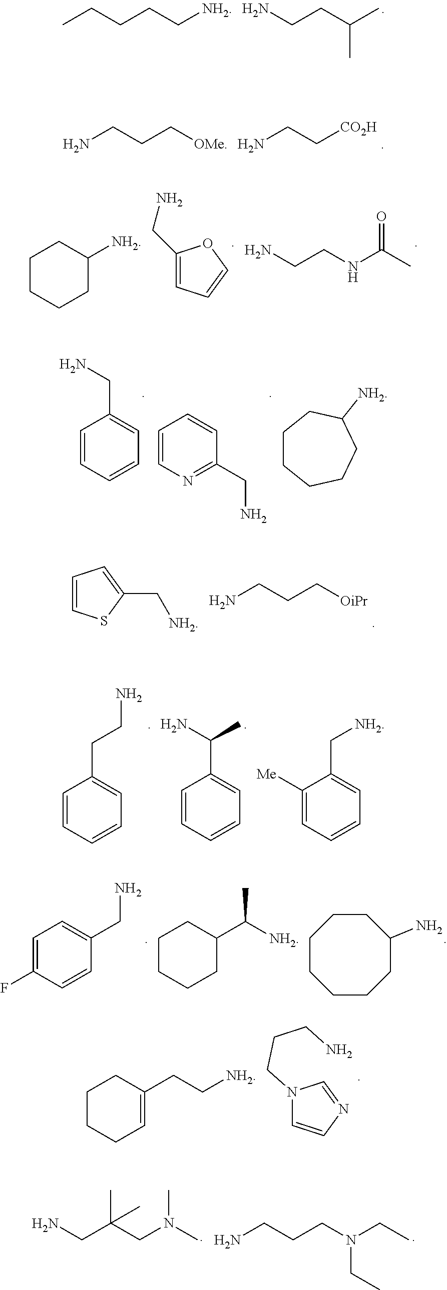

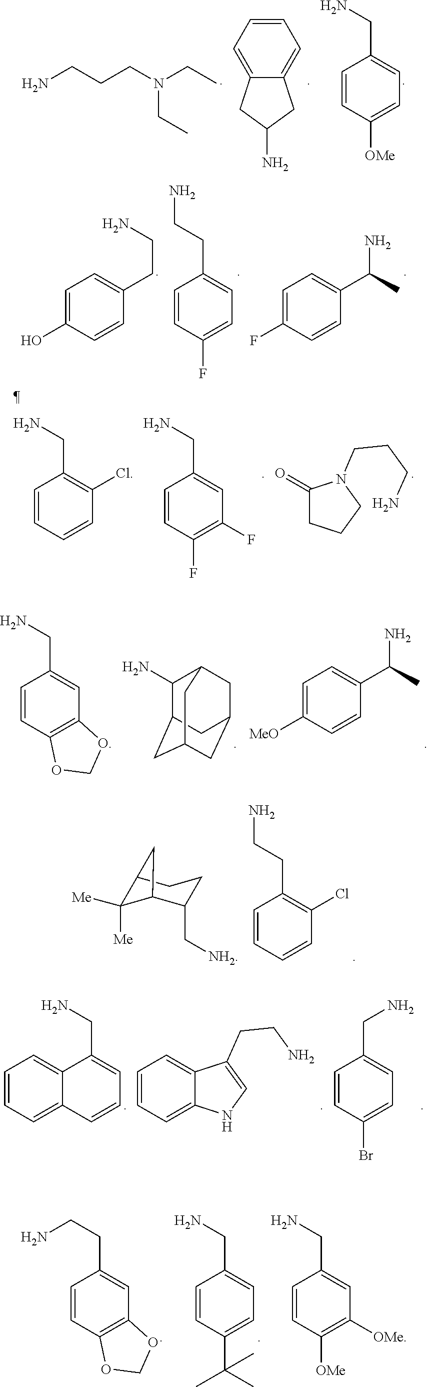

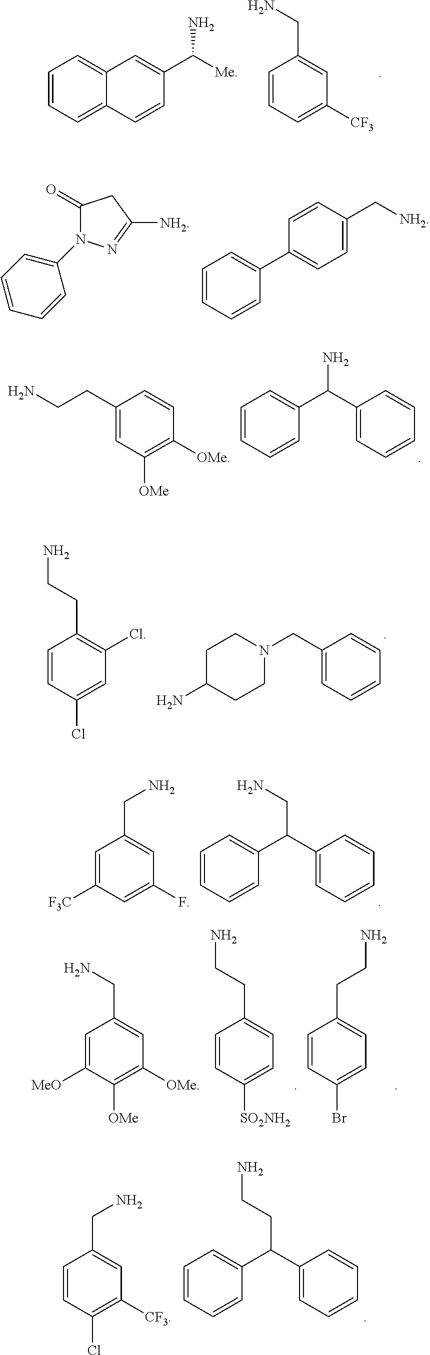

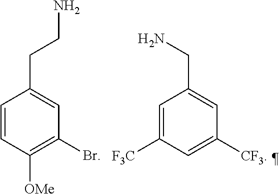

24. The method of claim 22, further comprising a polymer comprising one or more monomeric units, the one or more monomeric units comprising one or more peptoids.

25. The method of claim 24, further comprising wherein the monomeric unit comprises: dextran, amino acids, nucleic acids, synthetic molecules, organic or inorganic molecules, carbohydrates, variants or combinations thereof.

26. The method of claim 22, wherein an assay for detecting and diagnosing a disease or disorder comprises: immunoassays, ELISA assays, competitive ELISA assays, enzyme assays, bioassays, biochip assays, blots, hybridization assays, cell-based assays, high-throughput screening assays, chromatography, chemical assays, phage display assays, lab-on-a-chip, microfluidics based assays, microarrays, microchips, nanotube based assays, colorimetric assays, spectrophotometric assays or combinations thereof.

27. The method of claim 23, wherein the one or more peptoids comprise a detectable moiety, the detectable moiety comprising: a luminescent moiety, a chemiluminescent moiety, a fluorescence moiety, a bioluminescent moiety, an enzyme, a natural or synthetic moiety.

28. The method of claim 22, wherein a disease or disorder comprises: autoimmune diseases or disorders, cancer, inflammation, neurological diseases or disorders, infectious diseases or disorders, or combinations thereof.

29. A composition comprising two or more peptoids linked together, wherein the peptoids are of at least a 3-mer comprising a general structural formula (I): 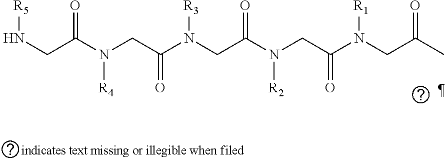
Wherein
R₁, R₂, R₃, R₄, R₅ independently comprise one or more groups derived from amines comprising: 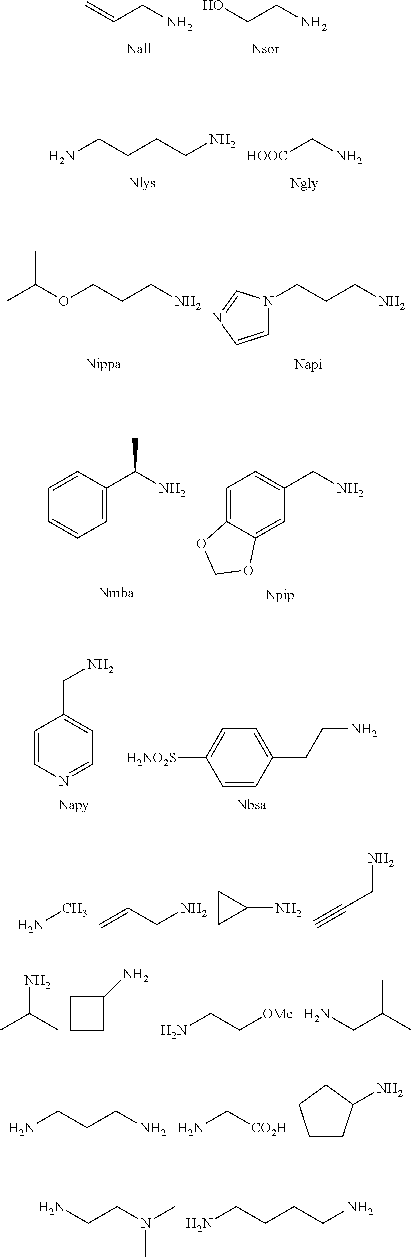 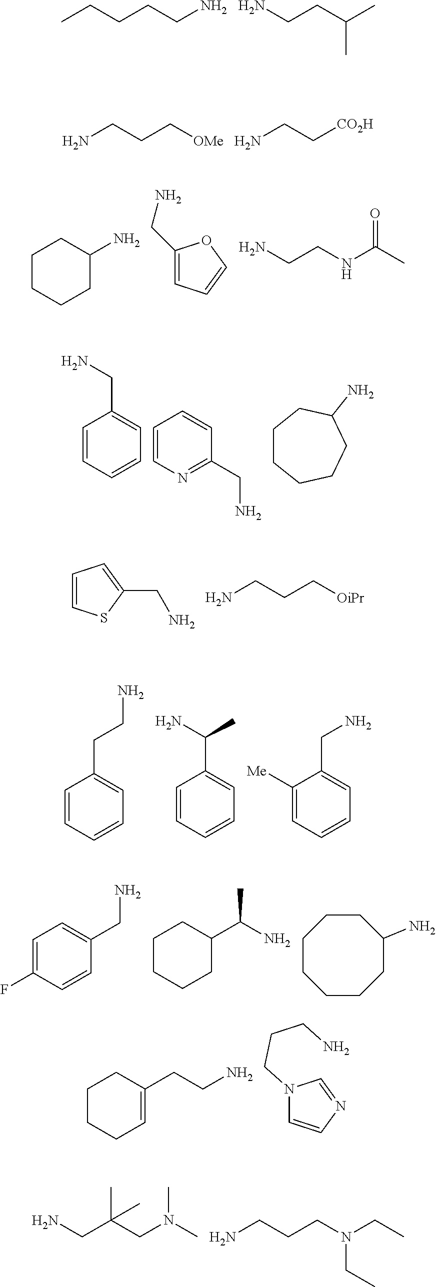 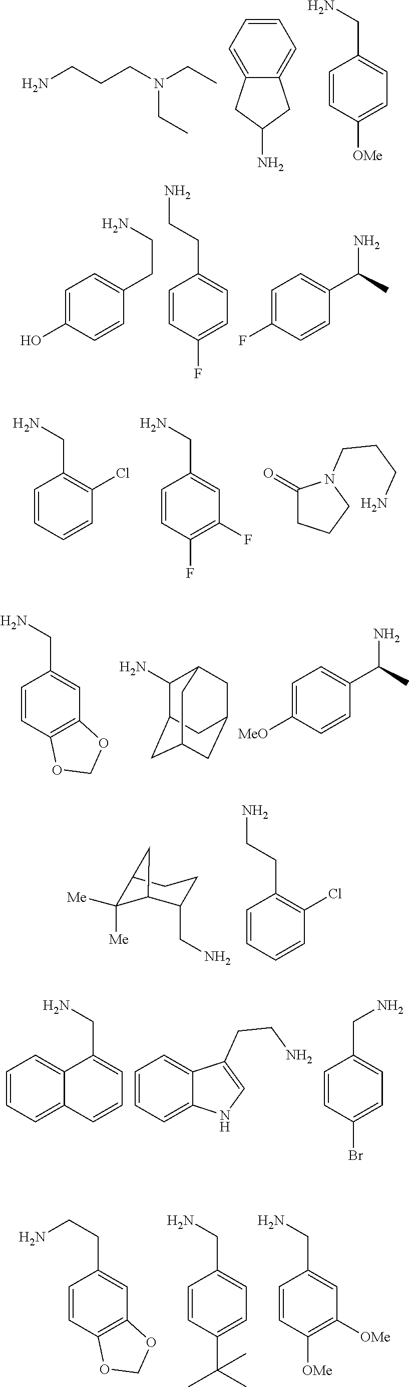 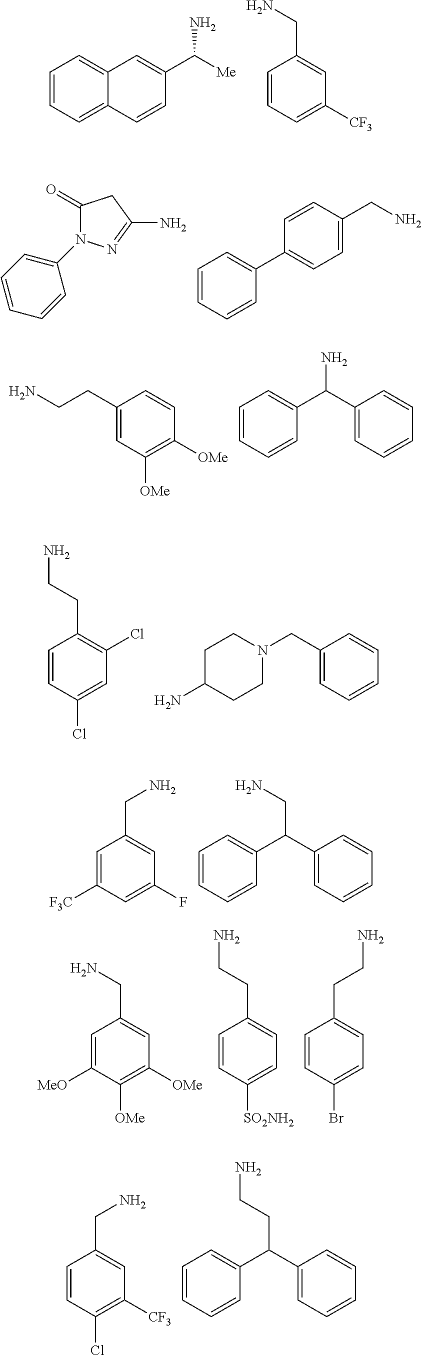 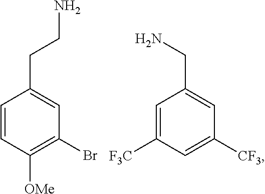
or combinations thereof.

30. The composition of claim 29, wherein the at least two peptoids are linked via linker molecules or via cross-linking agents.

31. The composition of claim 30, wherein a linking molecule comprises: alkyl groups, ether, polyether, alkyl amide linker, a peptide linker, a polypeptide linker, a modified peptide or polypeptide linker, a peptide nucleic acid (PNA) a Poly(ethylene glycol) (PEG) linker, a streptavidin-biotin or avidin-biotin linker, polyaminoacids (e.g. polylysine), functionalized PEG, polysaccharides, glycosaminoglycans, dendritic polymers PEG-chelant polymers, oligonucleotide linker, phospholipid derivatives, alkenyl chains, alkynyl chains, disulfide, or a combination thereof.
